(19) United States
(12) Patent Application Publication
(10) Pub. No.: US 2023/0288748 A1
(43) Pub. Date: Sep. 14, 2023

(54) LIGHT ABSORPTION FILTER, OPTICAL FILTER, SELF-LUMINOUS DISPLAY DEVICE, ORGANIC ELECTROLUMINESCENT DISPLAY DEVICE, AND LIQUID CRYSTAL DISPLAY DEVICE, AND MANUFACTURING METHOD FOR OPTICAL FILTER

(71) Applicant: FUJIFILM Corporation, Tokyo (JP)

(72) Inventor: Nobutaka FUKAGAWA, Kanagawa (JP)

(73) Assignee: FUJIFILM Corporation, Tokyo (JP)

(21) Appl. No.: 18/318,415

(22) Filed: May 16, 2023

Related U.S. Application Data
(63) Continuation of application No. PCT/JP2021/048408, filed on Dec. 24, 2021.

(30) Foreign Application Priority Data
Jan. 6, 2021 (JP) 2021-001136
Mar. 30, 2021 (JP) 2021-057023
Jul. 14, 2021 (JP) 2021-116334
Dec. 22, 2021 (JP) 2021-208371

(51) Int. Cl. G02F 1/1335; C09B 57/00; C09B 67/20; C09K 11/06; C09K 11/02; G03F 7/085; C07F 17/02; G03F 7/00

(52) U.S. Cl. CPC G02F 1/133516; C09B 57/007; C09B 67/0063; C09K 11/06; C09K 11/02; G03F 7/085; C07F 17/02; G03F 7/0007; G02F 2201/08; G02F 2202/04; C09K 2211/187; C09K 2211/1044; C09K 2211/1022; C09K 2211/1007; C09K 2211/1011

(57) ABSTRACT

There are provided a light absorption filter containing a resin, a dye having a main absorption wavelength band in a wavelength range of 400 to 700 nm, and a compound that generates a radical upon ultraviolet irradiation, where the resin is composed of a polymer having both a high affinity part and a low affinity part with respect to the dye and the compound that generates a radical upon ultraviolet irradiation, or a light absorption filter containing a dye having a main absorption wavelength band in a wavelength range of 400 to 700 nm and a resin composed of a polymer including a partial structure that generates a radical upon ultraviolet irradiation, an optical filter, a self-luminous display device, an organic electroluminescent display device, and a liquid crystal display device, in which this light absorption filter is used, as well as a manufacturing method for an optical filter.

### CROSS-REFERENCE TO RELATED APPLICATIONS

This application is a Continuation of PCT International Application No. PCT/JP2021/048408 filed on Dec. 24, 2021, which claims priority under 35 U.S.C. § 119 (a) to Japanese Patent Application No. 2021-001136 filed in Japan on Jan. 6, 2021, Japanese Patent Application No. 2021-057023 filed in Japan on Mar. 30, 2021, Japanese Patent Application No. 2021-116334 filed in Japan on Jul. 14, 2021, and Japanese Patent Application No. 2021-208371 filed in Japan on Dec. 22, 2021. Each of the above applications is hereby expressly incorporated by reference, in its entirety, into the present application.

### BACKGROUND OF THE INVENTION

### 1. Field of the Invention

The present invention relates to a light absorption filter, an optical filter, a self-luminous display device, an organic electroluminescent display device, and a liquid crystal display device, and a manufacturing method for an optical filter.

### 2. Description of the Related Art

As image display devices, an organic electroluminescence (OLED) display device, a liquid crystal display device, and the like have been used in recent years.

A liquid crystal display device is widely used year by year as a space-saving image display device with low power consumption. The liquid crystal display device is a non-light emitting element in which the liquid crystal panel itself displaying an image does not emit light, and thus the liquid crystal display device includes a backlight unit which is disposed on a rear surface of the liquid crystal panel and supplies light to the liquid crystal panel.

The OLED display device is a device that displays an image by utilizing the self-luminescence of the OLED element. Therefore, the OLED display device has advantages that a high contrast ratio, a high color reproducibility, a wide viewing angle, a high-speed responsiveness, and a reduction in thickness and weight can be achieved, as compared with various display devices such as a liquid crystal display device and a plasma display device. In addition to these advantages, in terms of flexibility, research and development are being actively carried out as a next-generation display device.

In the development of an image display device, it is known a technique of incorporating a light absorption filter as a configuration.

For example, in a liquid crystal display device, in a case where a white light emitting diode (LED) is used as a light source for a backlight unit, an attempt has been made to provide a light absorption filter in order to block light having unnecessary wavelengths emitted from the white LED. Further, in the OLED display device, an attempt has been made to provide a light absorption filter from the viewpoint of suppressing the reflection of external light.

In addition, in recent years, in a display (hereinafter, referred to as a “self-luminous display device”) using self-luminous light from an organic light emitting diode (OLED) element, a micro light emitting diode (LED) element, a mini LED element, or the like has been promoted, attempts have also been made to provide a light absorption filter for the intended purpose of suppressing a decrease in contrast in a bright place and improving color reproduction.

### SUMMARY OF THE INVENTION

As another form of the light absorption filter that is incorporated in the image display device, it is possible to conceive an optical filter having both a light absorptive portion having a light absorption effect and a portion in which the light absorption properties have been eliminated (hereinafter, also simply referred to as a “light absorption property-eliminated portion”), which is obtained by eliminating the light absorption properties of a desired portion.

For example, JP1997-286979A (JP-H9-286979A) discloses a photo-decolorizable composition containing a dye and a compound that changes the color development mechanism of the dye upon ultraviolet irradiation, and being faded or decolorized upon ultraviolet irradiation.

However, in a light absorption filter in which the photo-decolorizable composition described in JP1997-286979A (JP-H9-286979A) is used together with a resin, the decolorization rate upon ultraviolet irradiation is not so high. In addition, it has been found that, depending on the dye to be used, the dye is decomposed upon ultraviolet irradiation, and absorption derived from a new coloration structure (hereinafter, also referred to as “secondary absorption”) associated with this decomposition occurs.

In a form in which an optical filter is used by being incorporated in an image display device, it is required to set a specific portion of the optical filter as a light absorption property-eliminated portion, where the light absorption property-eliminated portion is required to have light absorption characteristics close to colorlessness.

That is, an object of the present invention is to provide a light absorption filter in which a desired portion becomes a light absorption property-eliminated portion upon ultraviolet irradiation, where the desired portion of the light absorption filter has an excellent decolorization rate upon ultraviolet irradiation and the secondary absorption associated with the dye decomposition upon ultraviolet irradiation hardly occurs.

In addition, another object of the present invention is to provide an optical filter using the above-described light absorption filter, where the optical filter includes an optical filter having a light absorptive portion and a light absorption property-eliminated portion at a desired position and to provide a self-luminous display device, an OLED display device, and a liquid crystal display device, which include this optical filter.

Further, another object of the present invention is to provide a manufacturing method for the optical filter.

As a result of diligent studies in consideration of the above problems, the inventors of the present invention found that the above-described excellent photoquenching property can be obtained by adopting a configuration of a light absorption filter that contains a dye having a main absorption in the visible light region, a compound that generate a radical upon ultraviolet irradiation, and a polymer having both a partial structure having a high affinity (hereinafter, also referred to as a “high affinity partial structure” or a “high affinity part”) and a partial structure having a low affinity (hereinafter, also referred to as a “low affinity partial structure” or “low affinity part”), with respect to the dye and the compound that generates a radical upon ultraviolet irradiation, or a configuration of a light absorption filter that contains a dye having a main absorption in a visible range and a resin composed of a polymer including a partial structure that generates a radical upon ultraviolet irradiation. Further studies have been carried out based on these findings, whereby the present invention has been completed.

That is, the above object has been achieved by the following means.
<1> A light absorption filter comprising:
a resin;
a dye that has a main absorption wavelength band in a wavelength range of 400 to 700 nm; and
a compound that generates a radical upon ultraviolet irradiation,
in which the resin is composed of a polymer having both a partial structure having a high affinity and a partial structure having a low affinity, with respect to the dye and the compound that generates a radical upon ultraviolet irradiation.
<2> The light absorption filter according to <1>, in which the dye includes a squaraine-based coloring agent represented by General Formula (1),

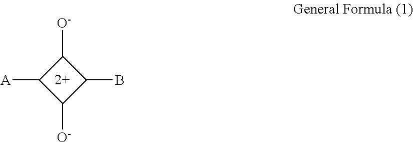

in the formula, A and B each independently represent an aryl group which may have a substituent, a heterocyclic group which may have a substituent, or —CH=G, where G represents a heterocyclic group which may have a substituent.
<3> The light absorption filter according to <1> or <2>, in which the polymer constituting the resin is a block copolymer.
<4> The light absorption filter according to any one of <1> to <3>, in which the partial structure having a high affinity includes a polystyrene structure.
<5> The light absorption filter according to any one of <1> to <4>, in which the partial structure having a low affinity includes a structure obtained by hydrogenating polydiene.
<6> A light absorption filter comprising:
a dye having a main absorption wavelength band in a wavelength range of 400 to 700 nm; and
a resin composed of a polymer including a partial structure that generates a radical upon ultraviolet irradiation.
<7> The light absorption filter according to <6>, in which the dye includes an electron-donating quencher-embedded coloring agent.
<8> The light absorption filter according to <7>, in which the electron-donating quencher-embedded coloring agent includes a squaraine-based coloring agent represented by General Formula (1A),

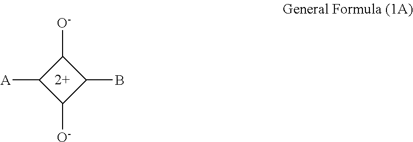

in the formula, A and B each independently represent an aryl group which may have a substituent, a heterocyclic group which may have a substituent, or —CH=G, where G represents a heterocyclic group which may have a substituent, provided that at least one of A or B includes an electron-donating quencher moiety.
<9> The light absorption filter according to any one of <6> to <8>, in which the polymer is a (meth)acrylic polymer having an aromatic ring in a side chain.
<10> The light absorption filter according to any one of <1> to <9>, in which the compound that generates a radical upon ultraviolet irradiation is a compound that generates a radical upon intramolecular cleavage of this compound, and the partial structure that generates a radical upon ultraviolet irradiation is a partial structure that generates a radical upon intramolecular cleavage of this partial structure.
<11> The light absorption filter according to any one of <1> to <8>, in which the compound that generates a radical upon ultraviolet irradiation is a compound that generates a radical by extracting a hydrogen atom from a compound that is present in a vicinity, and the partial structure that generates a radical upon ultraviolet irradiation is a partial structure that generates a radical by extracting a hydrogen atom from a compound that is present in a vicinity.
<12> The light absorption filter according to <11>, in which the compound that generates a radical by extracting a hydrogen atom from a compound that is present in a vicinity the compound is a benzophenone compound substituted with an alkoxy group, and the partial structure that generates a radical by extracting a hydrogen atom from a compound that is present in a vicinity of the partial structure is a partial structure derived from a benzophenone compound substituted with an alkoxy group.
<13> The light absorption filter according to any one of <1> to <12>, in which in the light absorption filter, the dye is chemically changed to be decolorized upon irradiation with light.
<14> An optical filter that is obtained by subjecting the light absorption filter according to any one of <1> to <13> to mask exposure by ultraviolet irradiation.
<15> A self-luminous display device, an organic electroluminescent display device, or a liquid crystal display device, comprising the optical filter according to <14>.
<16> The self-luminous display device, the organic electroluminescent display device, or the liquid crystal display device, according to <15>, in which a layer, which inhibits light absorption of the compound that generates a radical upon ultraviolet irradiation or light absorption of the partial structure that generates a radical upon ultraviolet irradiation, is provided on a viewer side with respect to the optical filter.
<17> A manufacturing method for an optical filter, comprising a step of irradiating the light absorption filter according to any one of <1> to <13> with an ultraviolet ray to carry out mask exposure.

In the present invention, in a case where there are a plurality of substituents, linking groups, and the like (hereinafter, referred to as substituents and the like) represented by specific reference numerals or formulae, or in a case where a plurality of substituents and the like are defined at the same time, the respective substituents and the like may be the same as or different from each other unless otherwise specified. The same applies to the definition of the number of substituents or the like. In addition, in a case where a plurality of substituents and the like are close to each other (particularly in a case where the substituents and the like are adjacent to each other), the substituents and the like may also be linked to each other to form a ring unless otherwise specified. In addition, unless otherwise specified, rings, for example, alicyclic rings, aromatic rings, and heterocyclic rings may be further fused to form a fused ring.

In the present invention, unless otherwise specified, the light absorption filter may contain one kind of each of components constituting the light absorption filter (a dye, a resin, and a compound that generates a radical upon ultraviolet irradiation, as well as another component that may be appropriately contained) or may contain two or more kinds thereof. The same applies to an optical filter produced by using the light absorption filter of the present invention.

Unless otherwise specified, the optical filter of the present invention can preferably apply the description regarding the light absorption filter of the present invention, except that it has a light absorption property-eliminated portion formed by ultraviolet irradiation.

In the present invention, in a case where an E type double bond and a Z type double bond are present in a molecule, the double bond may be any one thereof or may be a mixture thereof, unless otherwise specified.

In the present invention, the representation of a compound (including a complex) is used to mean not only the compound itself but also a salt thereof, and an ion thereof. In addition, it is meant to include those in which a part of the structure is changed as long as the effect of the present invention is not impaired. Furthermore, it is meant that a compound, which is not specified to be substituted or unsubstituted, may have any substituent as long as the effect of the present invention is not impaired. The same applies to the definition of a substituent or a linking group.

In addition, in the present invention, the numerical range indicated by using “to” means a range including the numerical values before and after “to” as the lower limit value and the upper limit value, respectively.

In the present invention, the “composition” includes a mixture in which the component concentration varies within a range in which a desired function is not impaired, in addition to a mixture in which the component concentration is constant (each component is uniformly dispersed).

In the present invention, the description of “having a main absorption wavelength band in a wavelength range of XX to YY nm” means that a wavelength at which the maximal absorption is exhibited (that is, the maximal absorption wavelength) is present in the wavelength range of XX to YY nm. Therefore, in a case where the maximal absorption wavelength is present in the above-described wavelength range, the entire absorption band including this wavelength may be in the above-described wavelength range or may also extend up to the outside of the above-described wavelength range. In addition, in a case where there are a plurality of maximal absorption wavelengths, it suffices that a maximal absorption wavelength at which the highest absorbance is exhibited is present in the above-described wavelength range. That is, the maximal absorption wavelength other than the maximal absorption wavelength at which the highest absorbance is exhibited may be present either inside or outside the above-described wavelength range of XX to YY nm.

In the light absorption filter of the present invention, a desired portion can be allowed to become a light absorption property-eliminated portion by ultraviolet irradiation, where the desired portion of the light absorption filter has an excellent decolorization rate upon ultraviolet irradiation and moreover, the secondary absorption associated with the dye decomposition upon ultraviolet irradiation hardly occurs.

In addition, the optical filter of the present invention, as well as the self-luminous display device, the OLED display device, and the liquid crystal display device of the present invention, which include this optical filter, can have a light absorptive portion and a light absorption property-eliminated portion at a desired position.

Further, according to the manufacturing method of the present invention, it is possible to obtain an optical filter having a light absorptive portion and a light absorption property-eliminated portion at a desired position.

### BRIEF DESCRIPTION OF THE DRAWINGS

FIG. 1 is a schematic view illustrating an outline of an embodiment of a liquid crystal display device having an optical filter according to the present invention.

### DESCRIPTION OF THE PREFERRED EMBODIMENTS

[Light Absorption Filter]

One form of a light absorption filter according to the embodiment of the present invention is composed of a resin, a dye that has a main absorption wavelength band in a wavelength range of 400 to 700 nm (hereinafter, also simply referred to as a “dye”), and a compound that generates a radical upon ultraviolet irradiation, where the resin is a polymer that has both a partial structure having a high affinity and a partial structure having a low affinity, with respect to both of the dye and the compound that generates a radical upon ultraviolet irradiation. It is preferable that the dye includes a squaraine-based coloring agent represented by General Formula (1) described below.

Another form of the light absorption filter according to the embodiment of the present invention is a light absorption filter that contains a resin composed of a polymer having a partial structure that generates a radical upon ultraviolet irradiation and contains a dye having a main absorption wavelength band in a wavelength range of 400 to 700 nm.

In the present invention, the main absorption wavelength band of a dye is the main absorption wavelength band of the dye, which is measured in the state of being a light absorption filter. Specifically, in Examples described later, it is measured in a state of being abase material-attached light absorption filter under the conditions described in the section of the absorbance of the light absorption filter.

In the light absorption filter according to the embodiment of the present invention, the “dye” is dispersed (preferably dissolved) in the resin to make the light absorption filter a layer that shows a specific absorption spectrum derived from the dye. The dispersion may be any type of dispersion, such as a random type or a regular type.

In addition, in a case where “the compound that generates a radical upon ultraviolet irradiation” described above is dispersed (preferably dissolved) in a resin, or a resin composed of a polymer into which a partial structure that generates a radical upon ultraviolet irradiation has been introduced is be formulated, a radical is generated upon ultraviolet irradiation. The dye can be faded and decolorized by a mechanism in which the generated radical reacts with the dye. As will be described later, a compound or partial structure that generates a radical upon ultraviolet irradiation can be allowed to become a compound or partial structure that generates a radical upon ultraviolet irradiation (hereinafter, also referred to as a “hydrogen abstraction type photoradical generator”) by extracting a hydrogen atom from a compound that is present in the vicinity of the compound or partial structure. In this case, the hydrogen abstraction type photoradical generator that has been excited upon irradiation with an ultraviolet ray abstracts a hydrogen atom (a hydrogen radical) of a dye present in the vicinity thereof to generate a dye having a radical. As a result, the dye can be faded and decolorized.

One form of a light absorption filter according to the embodiment of the present invention contains, in a resin, a dye having a main absorption wavelength band in a wavelength range of 400 to 700 nm and a compound that generates a radical upon ultraviolet irradiation. Another form of a light absorption filter according to the embodiment of the present invention contains a dye having a main absorption wavelength band in a wavelength range of 400 to 700 nm and a resin composed of a polymer including a partial structure that generates a radical upon ultraviolet irradiation. The light absorption filter according to the embodiment of the present invention having such a configuration is excellent in decolorization rate upon ultraviolet irradiation, and moreover, the secondary absorption associated with the dye decomposition hardly occurs, and the decolorization characteristics can be allowed to be close to colorlessness. The presumable reason for this is conceived to be as follows.

In the light absorption filter according to the embodiment of the present invention, the compound that generates a radical upon ultraviolet irradiation generates radical species upon ultraviolet irradiation, and the radical species directly or indirectly reacts with a dye to decompose the dye, whereby the dye is faded or decolorized. In addition, a hydrogen abstraction type photoradical generator that has been excited upon ultraviolet irradiation generates a dye that has a radical through a hydrogen abstraction reaction, and the active dye undergoes a reaction, decomposition, or the like, whereby the dye can be faded or decolorized. In particular, the squaraine-based coloring agent represented by General Formula (1) described later or the benzylidene-based or cinnamylidene-based coloring agent represented by General Formula (V) described later, which may be contained in the light absorption filter according to the embodiment of the present invention, is preferable from the viewpoint that it has a specific chemical structure, and thus the coloring agent can be decolorized with little secondary absorption associated with the dye decomposition.

Further, in one form of the light absorption filter according to the embodiment of the present invention, the resin is composed of a polymer having both a partial structure having a high affinity and a partial structure having a low affinity, with respect to the dye and the compound that generates a radical upon ultraviolet irradiation. As a result, in the light absorption filter according to the embodiment of the present invention, in this polymer, the radical species easily reacts with the dye by confining the dye and the compound that generates a radical upon ultraviolet irradiation in the high affinity part. Alternatively, a dye having a radical due to the hydrogen abstraction reaction is easily generated, and it is possible to exhibit an excellent decolorizing property under mild light irradiation conditions without increasing the reactivity by heating or the like.

In addition, in another form of the light absorption filter according to the embodiment of the present invention, since a resin composed of a polymer including a partial structure that generates a radical upon ultraviolet irradiation is contained, a radical is generated in the vicinity of the dye (that is, the partial structure that generates a radical upon ultraviolet irradiation) upon ultraviolet irradiation, and the radical easily reacts with a dye. Alternatively, a dye having a radical due to the hydrogen abstraction reaction is easily generated, and it is possible to exhibit an excellent decolorizing property under mild light irradiation conditions without increasing the reactivity by heating or the like.

As described above, in the light absorption filter according to the embodiment of the present invention, the dye is chemically changed to be decolorized upon irradiation with light (an ultraviolet ray). That is, the light absorption filter according to the embodiment of the present invention has a characteristic that the dye is chemically changed to be decolorized upon irradiation with light (an ultraviolet ray).

<Dye Having Main Absorption Wavelength Band in Wavelength Range of 400 to 700 nm>

Specific examples of the dye that is used in the present invention having a main absorption wavelength band in a wavelength range of 400 to 700 nm (hereinafter, also simply referred to as the “dye”) include tetraazaporphyrin (TAP)-based, squaraine (SQ)-based, cyanine (CY)-based, benzylidene-based, and cinnamylidene-based coloring agents (dyes).

The dye that can be contained in the light absorption filter according to the embodiment of the present invention may be one kind or two or more kinds.

The light absorption filter according to the embodiment of the present invention may also contain a dye other than the above dye.

Among these, the light absorption filter according to the embodiment of the present invention preferably contains, as the above-described dye, a squaraine-based coloring agent represented by General Formula (1) described later or a benzylidene-based or cinnamylidene-based coloring agent represented by General Formula (V) described later from the viewpoint that a secondary coloration structure associated with the decomposition of the dye is hardly generated. In a case where a coloring agent that hardly generates the secondary coloration structure associated with the dye decomposition as described above is used as the dye, the portion irradiated with ultraviolet light can be efficiently made colorless.

Further, the above-described dye is preferably the squaraine-based coloring agent represented by General Formula (1) described later or the benzylidene-based or cinnamylidene-based coloring agent represented by General Formula (V) described later from the viewpoint that the absorption waveform in the main absorption wavelength band is sharp. In a case where a coloring agent that has a sharp absorption waveform as described above is used as the dye, it is possible to minimize a decrease in the transmittance of the display light and prevent the reflection of external light.

That is, in a case where the squaraine-based coloring agent represented by General Formula (1) described later or the benzylidene-based or cinnamylidene-based coloring agent represented by General Formula (V) described later is used as the above-described dye, it is possible to suitably produce the optical filter according to the embodiment of the present invention by subjecting the light absorption filter according to the embodiment of the present invention to mask exposure by ultraviolet irradiation.

In the present invention, in the coloring agent represented by each General Formula, a cation is present in a delocalized manner, and thus a plurality of tautomer structures are present. Therefore, in the present invention, in a case where at least one tautomer structure of a certain coloring agent matches with each general formula, the certain coloring agent shall be a coloring agent represented by the general formula. Therefore, a coloring agent represented by a specific general formula can also be said to be a coloring agent having at least one tautomer structure that can be represented by the specific general formula. In the present invention, a coloring agent represented by a general formula may have any tautomer structure as long as at least one tautomer structure of the coloring agent matches with the general formula.

(1) Squaraine-Based Coloring Agent Represented by General Formula (1)

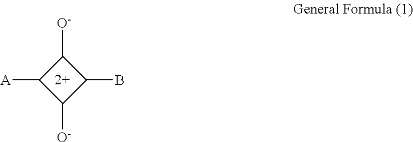

In General Formula (1), A and B each independently represent an aryl group which may have a substituent, a heterocyclic group which may have a substituent, or —CH=G. Here, G represents a heterocyclic group which may have a substituent.

The aryl group that can be employed as A or B is not particularly limited and may be a group consisting of a monocyclic ring or a group consisting of a fused ring. The aryl group preferably has 6 to 30 carbon atoms, more preferably 6 to 20 carbon atoms, and still more preferably 6 to 12 carbon atoms. Examples of the aryl group include groups respectively consisting of a benzene ring and a naphthalene ring, and a group consisting of a benzene ring is more preferable.

The heterocyclic group that can be employed as A or B is not particularly limited, and examples thereof include a group consisting of an aliphatic heterocyclic ring or an aromatic heterocyclic ring. A group consisting of an aromatic heterocyclic ring is preferable. Examples of the heteroaryl group that is an aromatic heterocyclic group include a heteroaryl group that can be employed as a substituent X described below. The aromatic heterocyclic group that can be employed as A or B is preferably a group of a 5-membered ring or a 6-membered ring and more preferably a group of a nitrogen-containing 5-membered ring. Specific examples thereof suitably include a group consisting of any of a pyrrole ring, a furan ring, a thiophene ring, an imidazole ring, a pyrazole ring, a thiazole ring, an oxazole ring, a triazole ring, an indole ring, an indolenine ring, an indoline ring, a pyridine ring, a pyrimidine ring, a quinoline ring, a benzothiazole ring, a benzoxazole ring, or a pyrazolotriazole ring. Among these, a group consisting of any of a pyrrole ring, a pyrazole ring, a thiazole ring, a pyridine ring, a pyrimidine ring, or a pyrazolotriazole ring is preferable. The pyrazolotriazole ring consists of a fused ring of a pyrazole ring and a triazole ring and may be a fused ring obtained by fusing at least one pyrazole ring and at least one triazole ring. Examples thereof include fused rings in General Formulae (4) and (5) described below.

A and B may be bonded to the squaric acid moiety (the 4-membered ring represented by General Formula (1)) at any portion (any ring-constituting atom) without particular limitation: however, they are preferably bonded at a carbon atom.

G in —CH=G that can be employed as A or B represents a heterocyclic group which may have a substituent, and examples thereof suitably include examples shown in the heterocyclic group that can be employed as A or B. Among these, a group consisting of any of a benzoxazole ring, a benzothiazole ring, an indoline ring, or the like is preferable.

At least one of A or B may have a hydrogen bonding group that forms an intramolecular hydrogen bond.

Each of A, B, and G may have the substituent X, and, in a case where A, B, or G has the substituent X, adjacent substituents may be bonded to each other to further form a ring structure. In addition, a plurality of substituents X may be present.

Examples of the substituent X include substituents that can be employed as R¹ in General Formula (2) described below. Specific examples thereof include a halogen atom, a cyano group, a nitro group, an alkyl group (including a cycloalkyl group), an alkenyl group, an alkynyl group, an aryl group, a heteroaryl group, an aralkyl group, and a ferrocenyl group, —OR¹⁰, —C(═O)R¹¹, —C(═O)OR¹², —OC(═O)R¹³, —NR¹⁴R¹⁵, —NHCOR¹⁶, —CONR¹⁷R¹⁸, —NHCONR¹⁹R²⁰, —NHCOOR²¹, —SR²², —SO₂R²³, —SO₃R²⁴, —NHSO₂R²⁵, and SO₂NR²⁶R²⁷. Further, it is also preferable that the substituent X has a quencher moiety described later, in addition to the ferrocenyl group.

In General Formula (1), R¹⁰ to R²⁷ each independently represent a hydrogen atom, an aliphatic group, an aromatic group, or a heterocyclic group. The aliphatic group and the aromatic group that can be employed as R¹⁰ to R²⁷ are not particularly limited, and appropriately selected from an alkyl group, a cycloalkyl group, an alkenyl group, and an alkynyl group which are classified as aliphatic groups, and an aryl group which is classified as an aromatic group, in the substituent that can be employed as R¹ in General Formula (2) described later. The heterocyclic group that can be employed as R¹⁰ to R²⁷ may be aliphatic or aromatic, and it can be appropriately selected from heteroaryl groups or heterocyclic groups that can be employed as R¹ in General Formula (2) described below.

It is noted that in a case where R¹² of —COOR¹² is a hydrogen atom (that is, a carboxy group), the hydrogen atom may be dissociated (that is, a carbonate group) or may be in a salt state. In addition, in a case where R²⁴ of —SO₃R²⁴ is a hydrogen atom (that is, a sulfo group), the hydrogen atom may be dissociated (that is, a sulfonate group) or may be in a salt state.

Examples of the halogen atom that can be employed as the substituent X include a fluorine atom, a chlorine atom, a bromine atom, and an iodine atom.

The alkyl group that can be employed as the substituent X preferably has 1 to 20 carbon atoms, more preferably 1 to 15 carbon atoms, and still more preferably 1 to 8 carbon atoms. The alkenyl group preferably has 2 to 20 carbon atoms, more preferably 2 to 12 carbon atoms, and still more preferably 2 to 8 carbon atoms. The alkynyl group preferably has 2 to 40 carbon atoms, more preferably 2 to 30 carbon atoms, and particularly preferably 2 to 25 carbon atoms. The alkyl group, the alkenyl group, and the alkynyl group each may be linear, branched, or cyclic, and they are preferably linear or branched.

The aryl group that can be employed as the substituent X includes a monocyclic group or a fused ring group. The aryl group preferably has 6 to 30 carbon atoms, more preferably 6 to 20 carbon atoms, and still more preferably 6 to 12 carbon atoms.

An alkyl portion in the aralkyl group that can be employed as the substituent X is the same as that in the alkyl group. An aryl moiety in the aralkyl group is the same as the aryl group described above. The aralkyl group preferably has 7 to 40 carbon atoms, more preferably 7 to 30 carbon atoms, and still more preferably 7 to 25 carbon atoms.

The heteroaryl group that can be employed as the substituent X includes a group consisting of a single ring or a fused ring, a group consisting of a single ring or a fused ring having 2 to 8 rings is preferable, and a group consisting of a single ring or a fused ring having 2 to 4 rings is more preferable. The number of heteroatoms constituting the ring of the heteroaryl group is preferably 1 to 3. Examples of the heteroatom constituting the ring of the heteroaryl group include a nitrogen atom, an oxygen atom, and a sulfur atom. The heteroaryl group is preferably a group consisting of a 5-membered ring or a 6-membered ring. The number of carbon atoms constituting the ring in the heteroaryl group is preferably 3 to 30, more preferably 3 to 18, and still more preferably 3 to 12. Examples of the heteroaryl group include each group consisting of any of a pyridine ring, a piperidine ring, a furan ring, a furfuran ring, a thiophene ring, a pyrrole ring, a quinoline ring, a morpholine ring, an indole ring, an imidazole ring, a pyrazole ring, a carbazole ring, a phenothiazine ring, a phenoxazine ring, an indoline ring, a thiazole ring, a pyrazine ring, a thiadiazine ring, a benzoquinoline ring, or a thiadiazole ring.

The ferrocenyl group that can be employed as the substituent X is preferably represented by General Formula (2M).

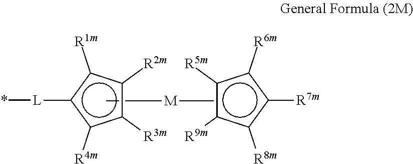

In General Formula (2M), L represents a single bond or a divalent linking group that does not conjugate with A, B, or G in General Formula (1). R^{1m} to R^{9m} each independently represent a hydrogen atom or a substituent. M represents an atom that can constitute a metallocene compound and represents Fe, Co, Ni, Ti, Cu, Zn, Zr, Cr, Mo, Os, Mn, Ru, Sn, Pd, Rh, V, or Pt. * represents a bonding site to A, B, or G.

In the present invention, in a case where L in General Formula (2M) is a single bond, a cyclopentadienyl ring directly bonded to A, B, or G (a ring having R^{1m} in General Formula (2M)) is not included in the conjugated structure which conjugates with A, B, or G.

The divalent linking group that can be employed as L is not particularly limited as long as it is a linking group that does not conjugate with A, B, or G, and it may have a conjugated structure in the inside thereof or at a cyclopentadiene ring side end part in General Formula (2M). Examples of the divalent linking group include an alkylene group having 1 to 20 carbon atoms, an arylene group having 6 to 20 carbon atoms, a divalent heterocyclic group obtained by removing two hydrogens from the heterocyclic ring, —CH═CH—, —CO—, —CS—, —NR—(R represents a hydrogen atom or a monovalent substituent), —O—, —S—, —SO₂—, or —N═CH—, or a divalent linking group formed by combining a plurality (preferably, 2 to 6) of these groups. The divalent linking group is preferably a group selected from the group consisting of an alkylene group having 1 to 8 carbon atoms, an arylene group having 6 to 12 carbon atoms, —CH═CH—, —CO—, —NR—(R is as described above), —O—, —S—, —SO₂—, and —N═CH—, or a divalent linking group in which two or more (preferably 2 to 6) selected from the above group are combined, and it is particularly preferably a group selected from the group consisting of an alkylene group having 1 to 4 carbon atoms, a phenylene group, —CO—, —NH—, —O—, and —SO₂—, or a linking group in which two or more (preferably 2 to 6) selected from the above group are combined. The divalent linking group combined is not particularly limited, and it is preferably a group containing —CO—, —NH—, —O—, or —SO₂—, and examples thereof include a linking group formed by combining two or more of —CO—, —NH—, —O—, or —SO₂—, or a linking group formed by combining at least one of —CO—, —NH—, —O—, or —SO₂— and an alkylene group or an arylene group. Examples of the linking group formed by combining two or more of —CO—, —NH—, —O—, or —SO₂— include —COO—, —OCO—, —CONH—, —NHCOO—, —NHCONH—, and —SO₂NH—. Examples of the linking group formed by combining at least one of —CO—, —NH—, —O—, or —SO₂— and an alkylene group or an arylene group include a group in which —CO—, —COO—, or —CONH— and an alkylene group or an arylene group are combined.

The substituent that can be employed as R is not particularly limited, and it has the same meaning as the substituent X which may be contained in A in General Formula (2).

L is preferably a single bond or a group selected from the group consisting of an alkylene group having 1 to 8 carbon atoms, an arylene group having 6 to 12 carbon atoms, —CH═CH—, —CO—, —NR— (R is as described above), —O—, —S—, —SO₂—, and —N═CH—, or a group in which two or more selected from the above group are combined.

L may have one or a plurality of substituents. The substituent which may be contained in L is not particularly limited, and for example, it has the same meaning as the substituent X. In a case where L has a plurality of substituents, the substituents bonded to adjacent atoms may be bonded to each other to further form a ring structure.

The alkylene group that can be employed as L may be linear, branched, or cyclic as long as the group has 1 to 20 carbon atoms, and examples thereof include methylene, ethylene, propylene, methylethylene, methylmethylene, dimethylmethylene, 1,1-dimethylethylene, butylene, 1-methylpropylene, 2-methylpropylene, 1,2-dimethylpropylene, 1,3-dimethylpropylene, 1-methylbutylene, 2-methylbutylene, 3-methylbutylene, 4-methylbutylene, 2,4-dimethylbutylene, 1,3-dimethylbutylene, pentylene, hexylene, heptylene, octylene, ethane-1,1-diyl, propane-2,2-diyl, cyclopropane-1,1-diyl, cyclopropane-1,2-diyl, cyclobutane-1,1-diyl, cyclobutane-1,2-diyl, cyclopentane-1,1-diyl, cyclopentane-1,2-diyl, cyclopentane-1,3-diyl, cyclohexane-1,1-diyl, cyclohexane-1,2-diyl, cyclohexane-1,3-diyl, cyclohexane-1,4-diyl, and methylcyclohexane-1,4-diyl.

In a case where a linking group containing at least one of —CO—, —CS—, —NR— (R is as described above), —O—, —S—, —SO₂—, or —N═CH— in the alkylene group is employed as L, the group such as —CO— may be incorporated at any site in the alkylene group, and the number of the groups incorporated is not particularly limited.

The arylene group that can be employed as L is not particularly limited as long as the group has 6 to 20 carbon atoms, and examples thereof include a group obtained by further removing one hydrogen atom from each group exemplified as the aryl group having 6 to 20 carbon atoms that can be employed as A in General Formula (1).

The heterocyclic group that can be employed as L is not particularly limited, and examples thereof include a group obtained by further removing one hydrogen atom from each group exemplified as the heterocyclic group that can be employed as A.

In General Formula (2M), the remaining partial structure excluding the linking group L corresponds to a structure (a metallocene structure portion) in which one hydrogen atom is removed from the metallocene compound. In the present invention, for the metallocene compound serving as the metallocene structure portion, a known metallocene compound can be used without particular limitation, as long as it is a compound conforming to the partial structure defined by General Formula (2M) (a compound in which a hydrogen atom is bonded instead of L). Hereinafter, the metallocene structure portion defined by General Formula (2M) will be specifically described.

In General Formula (2M), R^{1m} to R^{9m} each independently represent a hydrogen atom or a substituent. The substituents that can be employed as R^{1m} to R^{9m} are not particularly limited, and can be selected from, for example, the substituents that can be employed as R¹ in General Formula (3). R^{1m} to R^{9m} each are preferably a hydrogen atom, a halogen atom, an alkyl group, an acyl group, an alkoxy group, an amino group, or an amide group, more preferably a hydrogen atom, a halogen atom, an alkyl group, an acyl group, or an alkoxy group, still more preferably a hydrogen atom, a halogen atom, an alkyl group, or an acyl group, particularly preferably a hydrogen atom, a halogen atom, or an alkyl group, and most preferably a hydrogen atom.

As the alkyl group that can be employed as R^{1m} to R^{9m}, among the alkyl groups that can be employed as R¹, an alkyl group having 1 to 8 carbon atoms is preferable, and examples thereof include methyl, ethyl, propyl, isopropyl, butyl, sec-butyl, tert-butyl, isobutyl, pentyl, tert-pentyl, hexyl, octyl, and 2-ethylhexyl.

This alkyl group may have a halogen atom as a substituent. Examples of the alkyl group substituted with a halogen atom include, for example, chloromethyl, dichloromethyl, trichloromethyl, bromomethyl, dibromomethyl, tribromomethyl, fluoromethyl, difluoromethyl, trifluoromethyl, 2,2,2-trifluoroethyl, perfluoroethyl, perfluoropropyl, perfluorobutyl.

In addition, in the alkyl group that can be employed as R^{1m} or the like, at least one methylene group that forms a carbon chain may be substituted with —O— or —CO—. Examples of the alkyl group in which the methylene group is substituted with —O— include, for example, an alkyl group in which the end part methylene group of methoxy, ethoxy, propoxy, isopropoxy, isobutoxy, sec-butoxy, tert-butoxy, 2-methoxyethoxy, chloromethyloxy, dichloromethyloxy, trichloromethyloxy, bromomethyloxy, dibromomethyloxy, tribromomethyloxy, fluoromethyloxy, difluoromethyloxy, trifluoromethyloxy, 2,2,2-trifluoroethyloxy, perfluoroethyloxy, perfluoropropyloxy, or perfluorobutyloxy is substituted, and an alkyl group in which an internal methylene group of the carbon chain such as 2-methoxyethyl or the like is substituted. Examples of the alkyl group in which a methylene group is substituted with —CO— include acetyl, propionyl, monochloroacetyl, dichloroacetyl, trichloroacetyl, trifluoroacetyl, propane-2-one-1-yl, and butane-2-one-1-yl.

In General Formula (2M), M represents an atom that can constitute a metallocene compound, and represents Fe, Co, Ni, Ti, Cu, Zn, Zr, Cr, Mo, Os, Mn, Ru, Sn, Pd, Rh, V, or Pt. Among these, M is preferably Fe, Ti, Co, Ni, Zr, Ru, or Os, more preferably Fe, Ti, Ni, Ru, or Os, still more preferably Fe or Ti, and most preferably Fe.

The group represented by General Formula (2M) is preferably a group formed by combining preferred ones of L, R^{1m} to R^{9m}, and M. Examples thereof include a group formed by combining, as L, a single bond, or a group selected from the group consisting of an alkylene group having 2 to 8 carbon atoms, an arylene group having 6 to 12 carbon atoms, —CH═CH—, —CO—, —NR— (R is as described above), —O—, —S—, —SO₂—, and —N═CH—, or a group in which two or more selected from the above group are combined; as R^{1m} to R^{9m}, a hydrogen atom, a halogen atom, an alkyl group, an acyl group, or an alkoxy group; and as M, Fe.

The alkyl group, the alkenyl group, the alkynyl group, the aralkyl group, the aryl group, and the heteroaryl group which can be employed as the substituent X and the aliphatic group, the aromatic group, and the heterocyclic group which can be employed as R¹⁰ to R²⁷ each may further have a substituent or may be unsubstituted. The substituent which may be further contained therein is not particularly limited, and it is preferably a substituent selected from an alkyl group, an aryl group, an amino group, an alkoxy group, an aryloxy group, an aromatic heterocyclic oxy group, an acyl group, an alkoxycarbonyl group, an aryloxycarbonyl group, an acyloxy group, an acylamino group, an alkoxycarbonylamino group, an aryloxycarbonylamino group, a sulfonylamino group, an alkylthio group, an arylthio group, an aromatic heterocyclic thio group, a sulfonyl group, a ferrocenyl group, a hydroxy group, a mercapto group, a halogen atom, a cyano group, a sulfo group, or a carboxy group, and it is more preferably a substituent selected from an alkyl group, an aryl group, an alkoxy group, an aryloxy group, an aromatic heterocyclic oxy group, an acyl group, an alkoxycarbonyl group, an aryloxycarbonyl group, an acyloxy group, an alkylthio group, an arylthio group, an aromatic heterocyclic thio group, a sulfonyl group, a ferrocenyl group, a hydroxy group, a mercapto group, a halogen atom, a cyano group, a sulfo group, or a carboxy group. This group can be appropriately selected from the substituents that can be employed as R¹ in General Formula (2) described below.

One preferred embodiment of the coloring agent represented by General Formula (1) includes a coloring agent represented by General Formula (2).

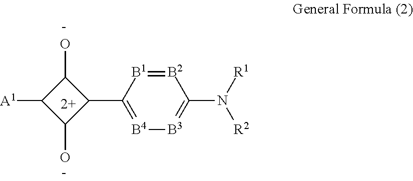

In General Formula (2), A¹ is the same as A in General Formula (1). Among these, a heterocyclic group which is a nitrogen-containing 5-membered ring is preferable.

In General Formula (2), R¹ and R² each independently represent a hydrogen atom or a substituent. R¹ and R² may be the same or different from each other, and they may be bonded together to form a ring.

The substituents that can be employed as R¹ and R² are not particularly limited, and examples thereof include an alkyl group (a methyl group, an ethyl group, a propyl group, an isopropyl group, a butyl group, a t-butyl group, an isobutyl group, a pentyl group, a hexyl group, an octyl group, a dodecyl group, a trifluoromethyl group, or the like), a cycloalkyl group (a cyclopentyl group, a cyclohexyl group, or the like), an alkenyl group (a vinyl group, an allyl group, or the like), an alkynyl group (an ethynyl group, a propargyl group, or the like), an aryl group (a phenyl group, a naphthyl group, or the like), a heteroaryl group (a furyl group, a thienyl group, a pyridyl group, a pyridazyl group, a pyrimidyl group, a pyrazyl group, a triazyl group, an imidazolyl group, a pyrazolyl group, a thiazolyl group, a benzoimidazolyl group, a benzoxazolyl group, a quinazolyl group, a phthalazyl group, or the like), a heterocyclic group (also referred to as a heterocyclic group, for example, a pyrrolidyl group, an imidazolidyl group, a morpholyl group, an oxazolidyl group, or the like), an alkoxy group (a methoxy group, an ethoxy group, a propyloxy group, or the like), a cycloalkoxy group (a cyclopentyloxy group, a cyclohexyloxy group, or the like), an aryloxy group (a phenoxy group, a naphthyloxy group, or the like), a heteroaryloxy group (an aromatic heterocyclic oxy group), an alkylthio a group (a methylthio group, an ethylthio group, a propylthio group, or the like), a cycloalkylthio group (a cyclopentylthio group, a cyclohexylthio group, or the like), an arylthio group (a phenylthio group, a naphthylthio group, or the like), a heteroarylthio group (an aromatic heterocyclic thio group), an alkoxycarbonyl group (a methyloxycarbonyl group, an ethyloxycarbonyl group, a butyloxycarbonyl group, an octyloxycarbonyl group, or the like), an aryloxycarbonyl group (a phenyloxycarbonyl group, a naphthyloxycarbonyl group, or the like), a phosphoryl group (dimethoxyphosphonyl or diphenylphosphoryl), a sulfamoyl a group (an aminosulfonyl group, a methylaminosulfonyl group, a dimethylaminosulfonyl group, a butylaminosulfonyl group, a cyclohexylaminosulfonyl group, an octylaminosulfonyl group, a phenylaminosulfonyl group, a 2-pyridylaminosulfonyl group, or the like), an ancyl a group (an acetyl group, an ethylcarbonyl group, a propylcarbonyl group, a cyclohexylcarbonyl group, an octylcarbonyl group, a 2-ethylhexylcarbonyl group, a phenylcarbonyl group, a naphthylcarbonyl group, a pyridylcarbonyl group, or the like), an acyloxy group (an acetyloxy group, an ethylcarbonyloxy group, a butylcarbonyloxy group, an octylcarbonyloxy group, a phenylcarbonyloxy group, or the like), an amide group (a methylcarbonylamino group, an ethylcarbonylamino group, a dimethylcarbonylamino group, a propylcarbonylamino group, a pentylcarbonylamino group, a cyclohexylcarbonylamino group, a 2-ethylhexylcarbonylamino group, an octylcarbonylamino group, a dodecylcarbonylamino group, a phenylcarbonylamino group, a naphthylcarbonylamino group, or the like), a sulfonylamide group (a methylsulfonylamino group, an octylsulfonylamino group, a 2-ethylhexylsulfonylamino group, a trifluoromethylsulfonylamino group, or the like), a carbamoyl group (an aminocarbonyl group, a methylaminocarbonyl group, a dimethylaminocarbonyl group, a propylaminocarbonyl group, a pentylaminocarbonyl group, a cyclohexylaminocarbonyl group, an octylaminocarbonyl group, a 2-ethylhexylaminocarbonyl group, a dodecylaminocarbonyl group, a phenylaminocarbonyl group, a naphthylaminocarbonyl group, a 2-pyridylaminocarbonyl group, or the like), a ureido group (a methylureido group, an ethylureido group, a pentylureido group, a cyclohexylureido group, an octylureido group, a dodecylureido group, a phenylureido group, a naphthylureido group, a 2-pyridylaminoureido group, or the like), an alkylsulfonyl group (a methylsulfonyl group, an ethylsulfonyl group, a butylsulfonyl group, a cyclohexylsulfonyl group, a 2-ethylhexylsulfonyl group, or the like), an arylsulfonyl group (a phenylsulfonyl group, a naphthylsulfonyl group, a 2-pyridylsulfonyl group, or the like), an amino group (an amino group, an ethylamino group, a dimethylamino group, a butylamino group, a dibutylamino group, a cyclopentylamino group, a 2-ethylhexylamino group, a dodecylamino group, an anilino group, a naphthylamino group, a 2-pyridylamino group, or the like), an alkylsulfonyloxy group (methanesulfonyloxy), a cyano group, a nitro group, halogen atoms (a fluorine atom, a chlorine atom, a bromine atom, or the like), and a hydroxy group.

Among these, an alkyl group, an alkenyl group, an aryl group, or a heteroaryl group is preferable, an alkyl group, an aryl group, or a heteroaryl group is more preferable, and an alkyl group is still more preferable.

The substituent that can be employed as R¹ and R² may further have a substituent. Examples of the substituent which may be further contained therein include the substituent that can be employed as R¹ and R², and the substituent X which may be contained in A, B, and G in General Formula (1) described above. In addition, R¹ and R² may be bonded to each other to form a ring, and R¹ or R² and the substituent of B² or B³ may be bonded to each other to form a ring.

The ring that is formed in this case is preferably a heterocyclic ring or a heteroaryl ring, and it is preferably a 5-membered ring or a 6-membered ring although the size of the ring to be formed is not particularly limited. Further, the number of rings to be formed is not particularly limited, and it may be one or may be two or more. Examples of the form in which two or more rings are formed include a form in which the substituents of R¹ and B² and the substituents of R² and B³ are respectively bonded to each other to form two rings.

In General Formula (2), B¹, B², B³, and B⁴ each independently represent a carbon atom or a nitrogen atom. The ring including B¹, B², B³, and B⁴ is an aromatic ring. It is preferable that at least two or more of B¹ to B⁴ are a carbon atom, and it is more preferable that all of B¹ to B⁴ are a carbon atom.

The carbon atom that can be employed as B¹ to B⁴ has a hydrogen atom or a substituent. Among carbon atoms that can be employed as B¹ to B⁴, the number of carbon atoms having a substituent is not particularly limited; however, it is preferably zero, one, or two, and more preferably one. Particularly, it is preferable that B¹ and B⁴ are a carbon atom and at least one of them has a substituent.

The substituent possessed by the carbon atom that can be employed as B¹ to B⁴ is not particularly limited, and examples thereof include the above-described substituents that can be employed as R¹ and R². Among these, it is preferably an alkyl group, an alkoxy group, an alkoxycarbonyl group, an aryl group, an acyl group, an amide group, a sulfonylamide group, a carbamoyl group, an alkylsulfonyl group, an arylsulfonyl group, an amino group, a cyano group, a nitro group, a halogen atom, or a hydroxy group, and it is more preferably an alkyl group, an alkoxy group, an alkoxycarbonyl group, an aryl group, an acyl group, an amide group, a sulfonylamide group, a carbamoyl group, an amino group, a cyano group, a nitro group, a halogen atom, or a hydroxy group.

The substituent possessed by the carbon atom that can be adopted as B¹ to B⁴ may further have a substituent. The substituents that may be further possessed by the carbon atom include the substituent which may be further contained in R¹ and R² in General Formula (2) described above and the substituent X which may be contained in A, B, and G in General Formula (1) described above, where a ferrocenyl group is preferable.

Examples of the substituent that can be possessed by the carbon atom that can be employed as B¹ and B⁴ still more preferably include an alkyl group, an alkoxy group, a hydroxy group, an amide group, a sulfonylamide group, or a carbamoyl group, and particularly preferably an alkyl group, an alkoxy group, a hydroxy group, an amide group, or a sulfonylamide group, and a hydroxy group, an amide group, or a sulfonylamide group is most preferable. The substituent possessed by the carbon atom that can be employed as B¹ and B⁴ may further have a ferrocenyl group.

It is still more preferable that the substituent that can be possessed by the carbon atom that can be employed as B² and B³ is an alkyl group, an alkoxy group, an alkoxycarbonyl group, an acyl group, an amino group, a cyano group, a nitro group, or a halogen atom, and it is particularly preferable that the substituent as any one of B² or B³ is an electron withdrawing group (for example, an alkoxycarbonyl group, an acyl group, a cyano group, a nitro group, or a halogen atom).

The coloring agent represented by General Formula (2) is preferably a coloring agent represented by any of General Formulae (3), (4), or (5).

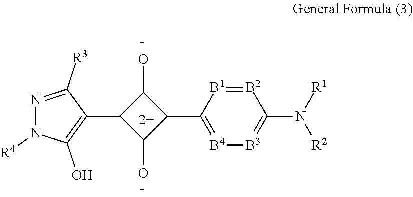

In General Formula (3), R¹ and R² each independently represent a hydrogen atom or a substituent, and they respectively have the same meanings as R¹ and R² in General Formula (2), where the same applies to the preferred ranges thereof.

In General Formula (3), B¹ to B⁴ each independently represent a carbon atom or a nitrogen atom, and they have respectively the same meanings as B¹ to B⁴ in General Formula (2), where the same applies to the preferred ranges thereof.

In General Formula (3), R³ and R⁴ each independently represent a hydrogen atom or a substituent. The substituent that can be employed as R³ and R⁴ is not particularly limited, and examples thereof include the same ones as the substituents that can be employed as R¹ and R².

However, the substituent that can be employed as R³ is preferably an alkyl group, an alkoxy group, an amino group, an amide group, a sulfonylamide group, a cyano group, a nitro group, an aryl group, a heteroaryl group, a heterocyclic group, an alkoxycarbonyl group, a carbamoyl group, or a halogen atom, more preferably an alkyl group, an aryl group, or an amino group, and still more preferably an alkyl group. This substituent that can be employed as R³ may further have a ferrocenyl group.

The substituent that can be employed as R⁴ is preferably an alkyl group, an aryl group, a heteroaryl group, a heterocyclic group, an alkoxy group, an alkoxycarbonyl group, an acyl group, an acyloxy group, an amide group, a carbamoyl group, an amino group, or a cyano group, more preferably an alkyl group, an alkoxycarbonyl group, an acyl group, a carbamoyl group, or an aryl group, and still more preferably an alkyl group.

The alkyl group that can be employed as R³ and R⁴ may be either linear, branched, or cyclic, and it is preferably linear or branched. The alkyl group preferably has 1 to 12 carbon atoms and more preferably 1 to 8 carbon atoms. An example of the alkyl group is preferably a methyl group, an ethyl group, an n-propyl group, an isopropyl group, a t-butyl group, a 2-ethylhexyl group, or a cyclohexyl group, and more preferably a methyl group or a t-butyl group.

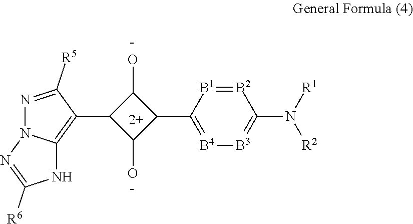

In General Formula (4), R¹ and R² each independently represent a hydrogen atom or a substituent, and they respectively have the same meanings as R¹ and R² in General Formula (2), where the same applies to the preferred ranges thereof.

In General Formula (4), B¹ to B⁴ each independently represent a carbon atom or a nitrogen atom, and they have respectively the same meanings as B¹ to B⁴ in General Formula (2), where the same applies to the preferred ranges thereof.

In General Formula (4), R⁵ and R⁶ each independently represent a hydrogen atom or a substituent. The substituent that can be employed as R⁵ and R⁶ is not particularly limited, and examples thereof include the same ones as the substituents that can be employed as R¹ and R².

However, the substituent that can be employed as R⁵ is preferably an alkyl group, an alkoxy group, an aryloxy group, an amino group, a cyano group, an aryl group, a heteroaryl group, a heterocyclic group, an acyl group, an acyloxy group, an amide group, a sulfonylamide group, an ureido group, or a carbamoyl group, more preferably an alkyl group, an alkoxy group, an acyl group, an amide group, or an amino group, and still more preferably an alkyl group.

The alkyl group that can be employed as R⁵ has the same meaning as the alkyl group that can be employed as R³ in General Formula (3), and the same applies to the preferred range thereof.

In General Formula (4), the substituent that can be employed as R⁶ is preferably an alkyl group, an alkenyl group, an aryl group, a heteroaryl group, a heterocyclic group, an alkoxy group, a cycloalkoxy group, an aryloxy group, an alkoxycarbonyl group, an acyl group, an acyloxy group, an amide group, a sulfonylamide group, an alkylsulfonyl group, an arylsulfonyl group, a carbamoyl group, an amino group, a cyano group, a nitro group, or a halogen atom, more preferably an alkyl group, an aryl group, a heteroaryl group, or a heterocyclic group, and still more preferably an alkyl group or an aryl group.

The alkyl group that can be employed as R⁶ has the same meaning as the alkyl group that can be employed as R⁴ in General Formula (3), and the same applies to the preferred range thereof.

The aryl group that can be employed as R⁶ is preferably an aryl group having 6 to 12 carbon atoms, and more preferably a phenyl group. This aryl group may have a substituent, and examples of such a substituent include a group included in the following substituent group A, and an alkyl group, a sulfonyl group, an amino group, an acylamino group, a sulfonylamino group, or the like, which have 1 to 10 carbon atoms, is particularly preferable. This substituent may further have a substituent. Specifically, the substituent is preferably an alkylsulfonylamino group.

—Substituent Group A—

A halogen atom, an alkyl group, an alkenyl group, an alkynyl group, an aryl group, a heterocyclic group, a cyano group, a hydroxy group, a nitro group, a carboxy group, an alkoxy group, an aminooxy group, an aryloxy group, a silyloxy group, a heterocyclic oxy group, an acyloxy group, a carbamoyloxy group, an amino group, an acylamino group, an aminocarbonylamino group, an alkoxycarbonylamino group, an aryloxycarbonylamino group, a sulfamoylamino group, a sulfonylamino group (including an alkyl or arylsulfonylamino group), a mercapto group, an alkylthio group, an arylthio group, a heterocyclic thio group, a sulfamoyl group, a sulfo group, an alkyl or arylsulfonyl group, a sulfonyl group (including an alkyl or arylsulfinyl group), an acyl group, an aryloxycarbonyl group, an alkoxycarbonyl group, a carbamoyl group, an aryl or heterocyclic azo group, an imide group, a phosphino group, a phosphinyl group, a phosphinyloxy group, a phosphinylamino group, a silyl group, and the like.

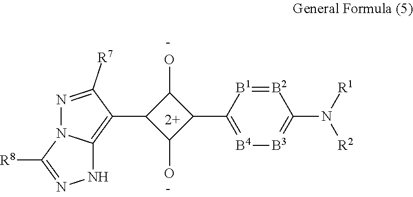

In General Formula (5), R¹ and R² each independently represent a hydrogen atom or a substituent, and they respectively have the same meanings as R¹ and R² in General Formula (2), where the same applies to the preferred ranges thereof.

In General Formula (5), B¹ to B⁴ each independently represent a carbon atom or a nitrogen atom, and they have respectively the same meanings as B¹ to B⁴ in General Formula (2), where the same applies to the preferred ranges thereof.

In General Formula (5), R⁷ and R⁸ each independently represent a hydrogen atom or a substituent. The substituent that can be employed as R⁷ and R⁸ is not particularly limited, and examples thereof include the same ones as the substituents that can be employed as R¹ and R².

However, the preferred range, the more preferred range, and the still more preferred range of the substituent that can be employed as R⁷ are the same as those of the substituent that can be employed as R⁵ in General Formula (4). The alkyl group that can be employed as R⁵ has the same meaning as the alkyl group that can be employed as R³, and the same applies to the preferred range thereof.

In General Formula (5), the preferred range, the more preferred range, and the still more preferred range of the substituent that can be employed as R⁸ are the same as those of the substituent that can be employed as R⁶ in General Formula (4). The preferred ranges of the alkyl group and the aryl group that can be employed as R⁸ have the same meaning as the alkyl group and the aryl group that can be employed as R⁶ in General Formula (4), where the same applies to the preferred ranges thereof.

As the squaraine-based coloring agent that is used for the above dye, any squaraine coloring agent represented by any of General Formulae (1) to (5) can be used without particular limitation. Examples thereof include compounds described in, for example, JP2006-160618A, WO2004/005981A, WO2004/007447A, Dyes and Pigment, 2001, 49, p. 161 to 179, WO2008/090757A, WO2005/121098A, and JP2008-275726A.

Hereinafter, specific examples of the coloring agent represented by any of General Formula (1) to General Formula (5) will be shown. However, the present invention is not limited thereto.

In the following specific examples, Me represents methyl, Et represents ethyl, Bu represents butyl, and Ph represents phenyl, respectively.

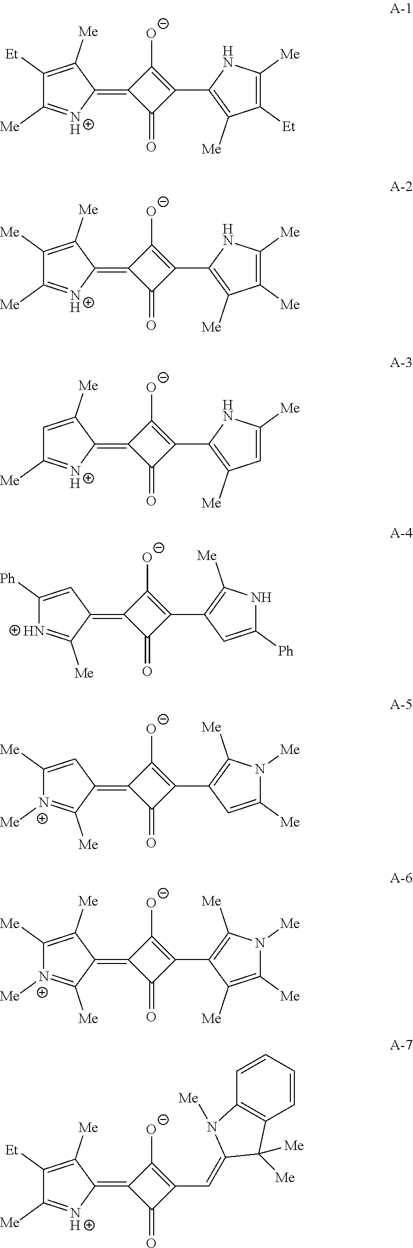

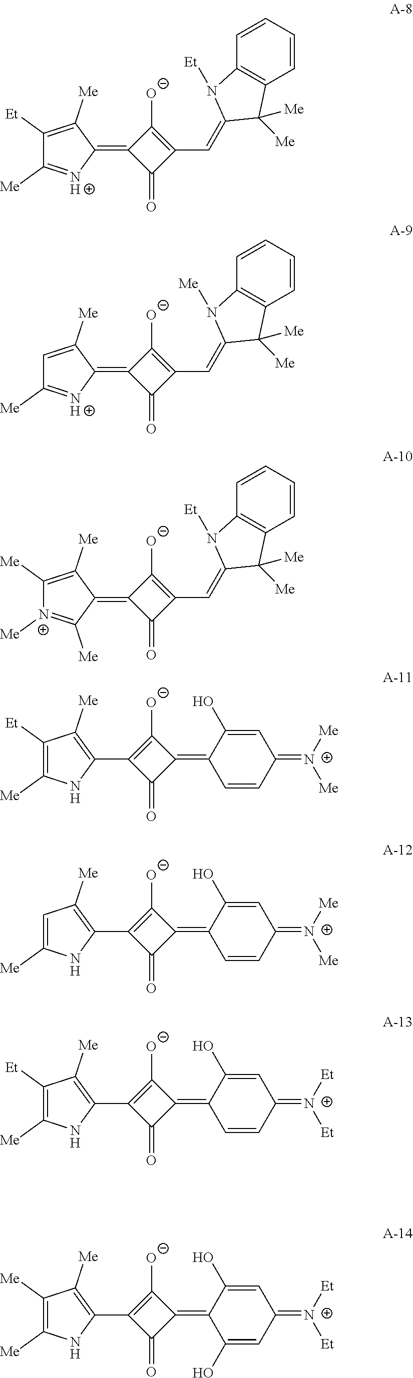

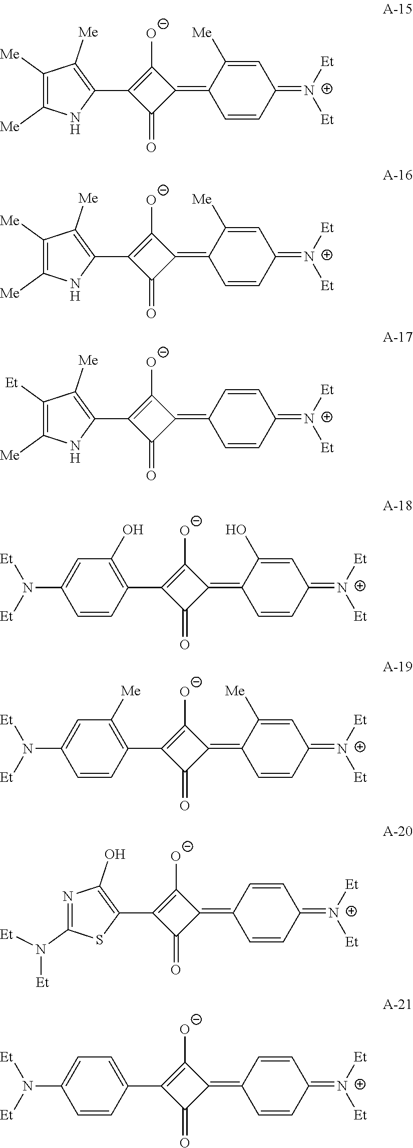

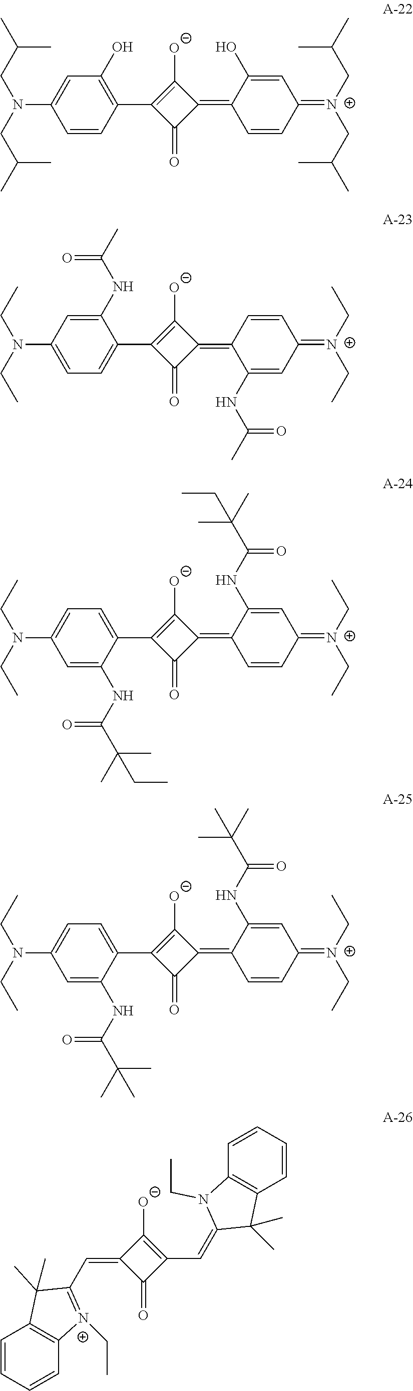

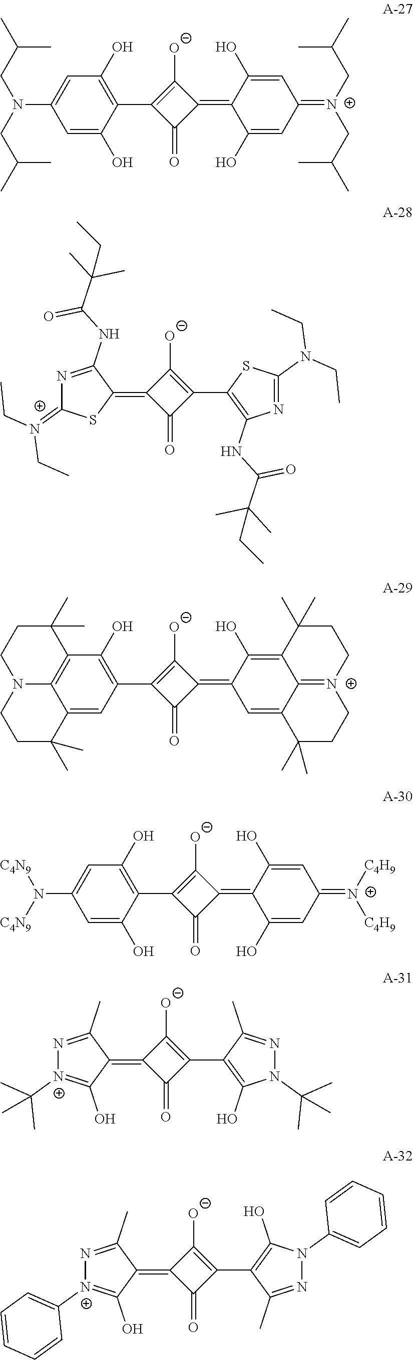

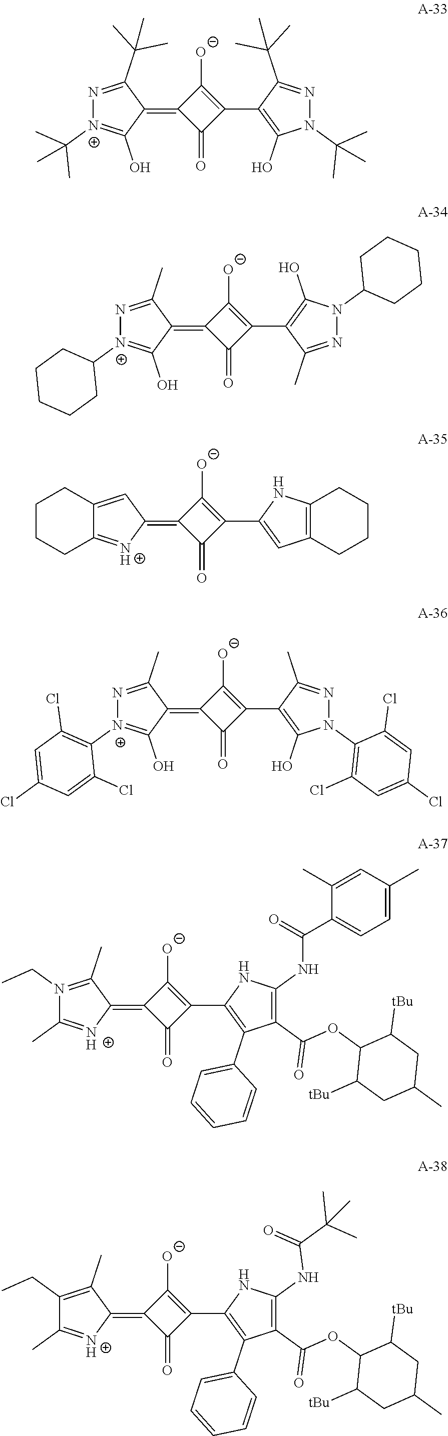

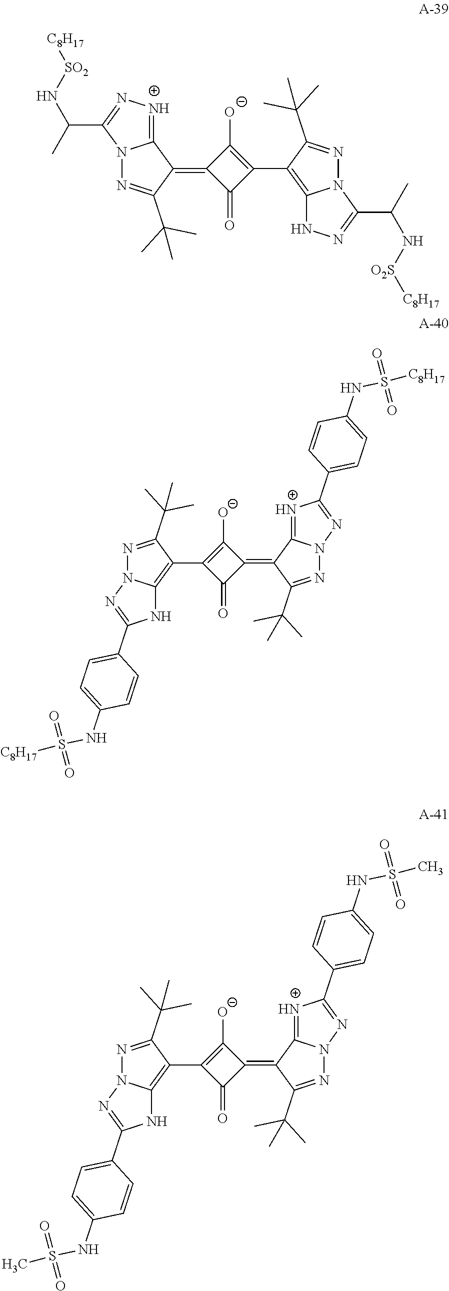

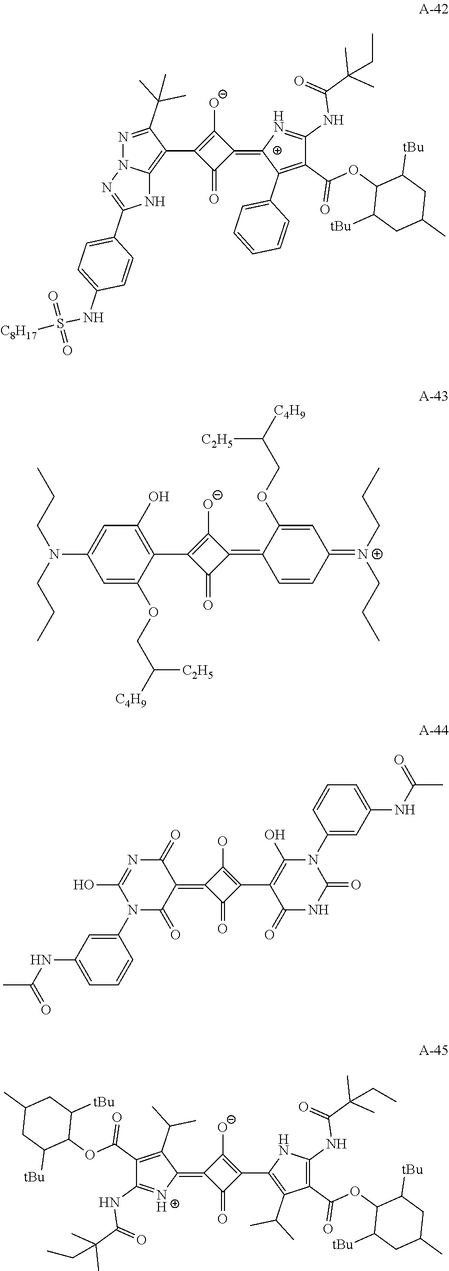

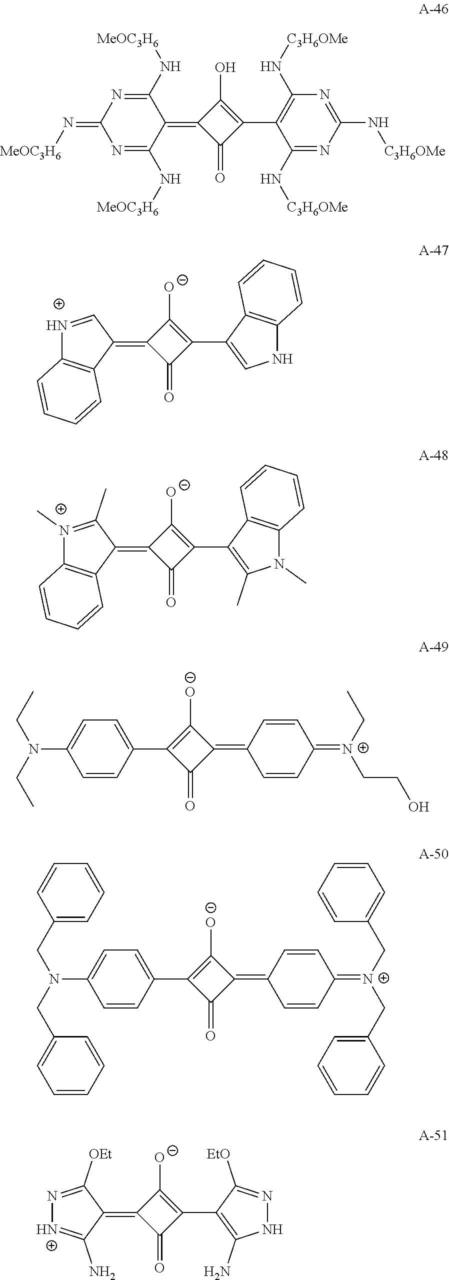

In addition to the above-described specific examples, specific examples of the coloring agents represented by any of General Formulae (3) to (5) will be shown. The substituent B in the following tables represents the following structures. In the following structures and the following tables, Me represents methyl, Et represents ethyl, i-Pr represents i-propyl, Bu represents n-butyl, t-Bu represents t-butyl, and Ph represents phenyl, respectively. In the following structures, * indicates a bonding site to a 4-membered carbon ring in each General Formula.

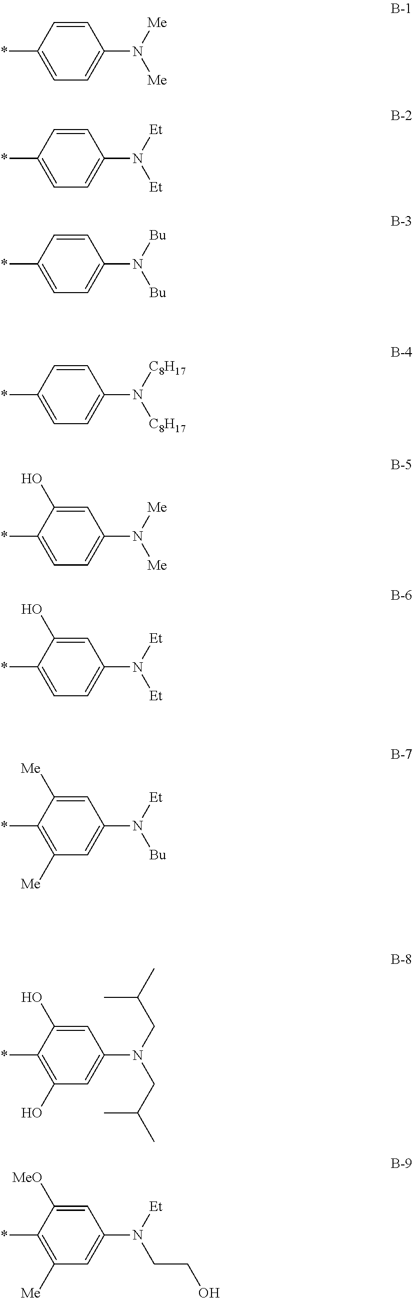

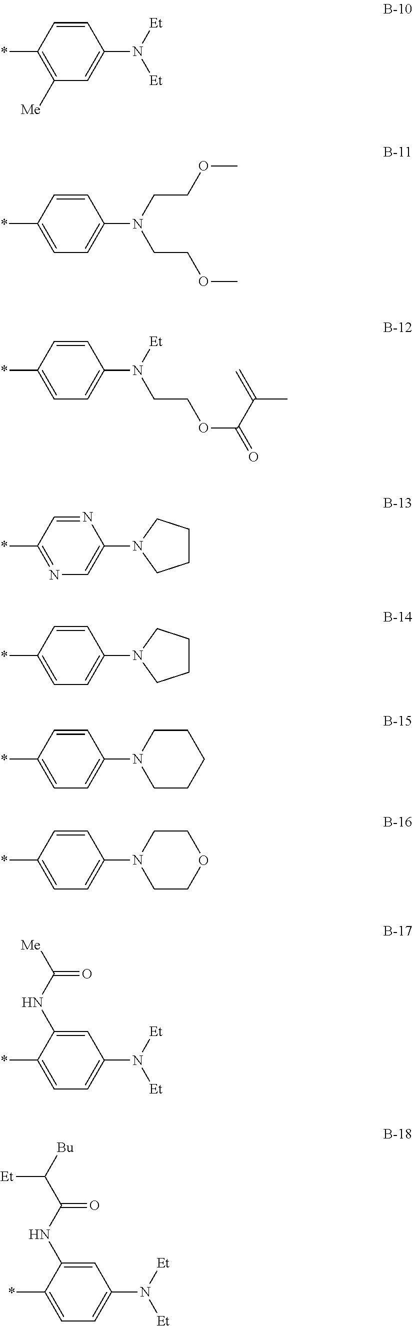

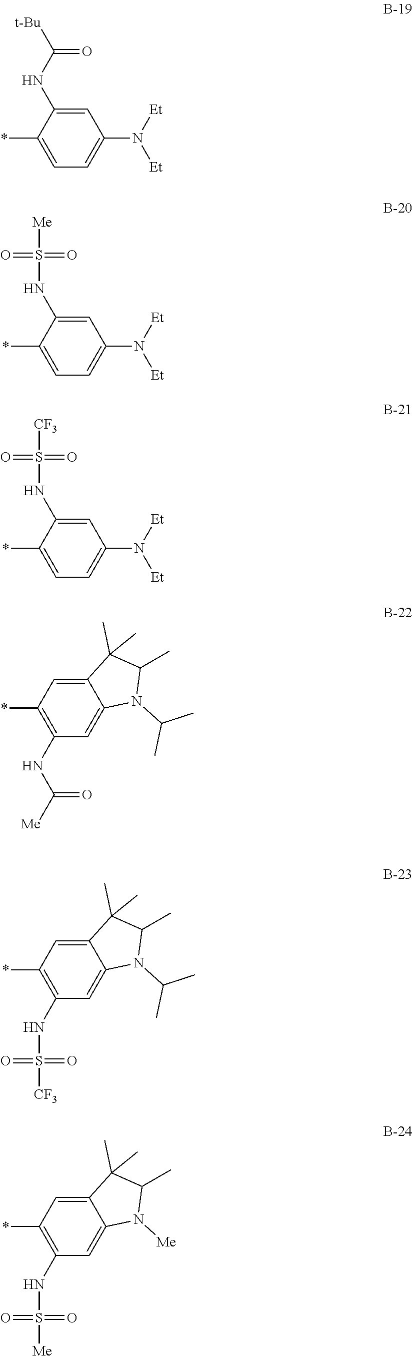

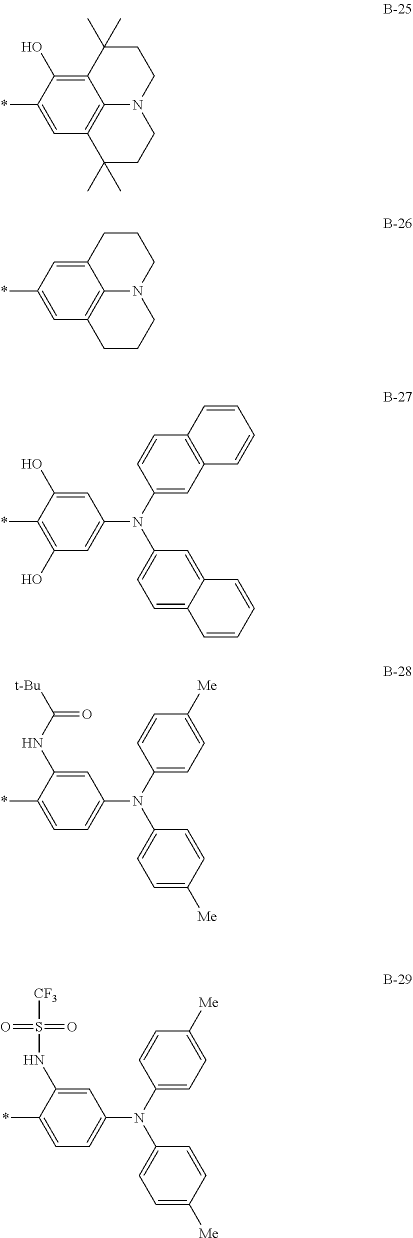

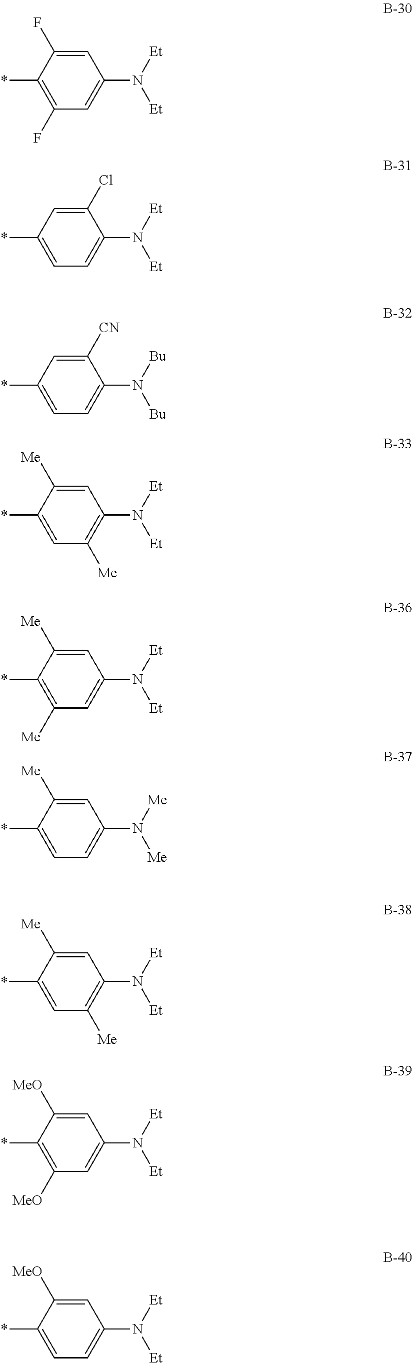

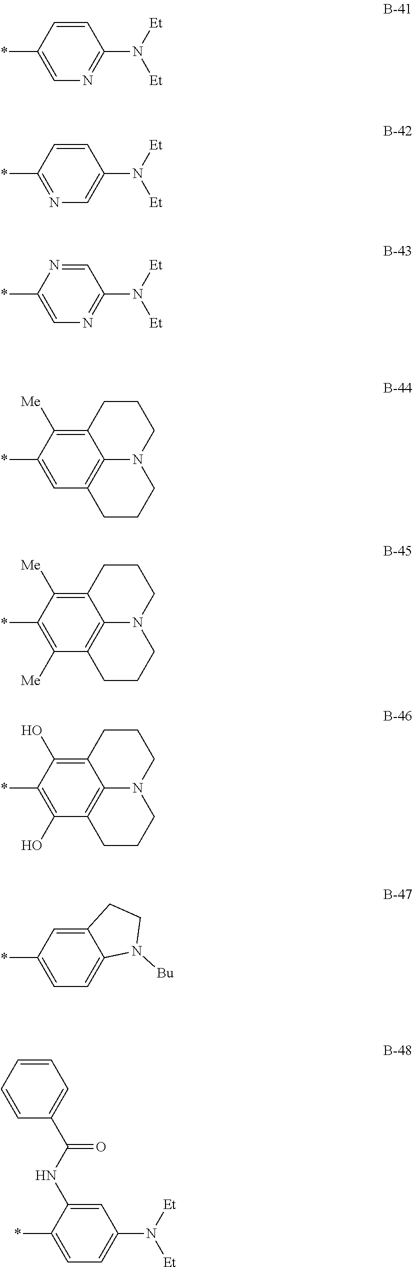

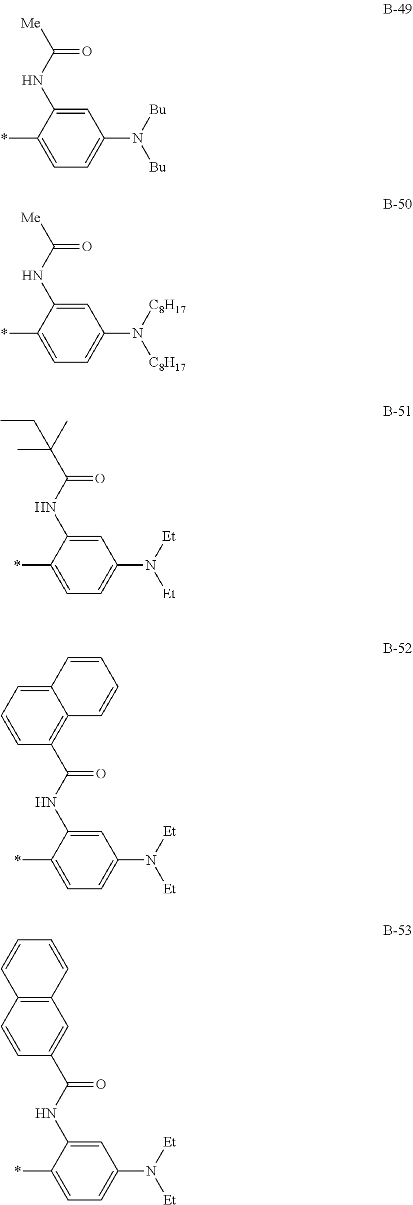

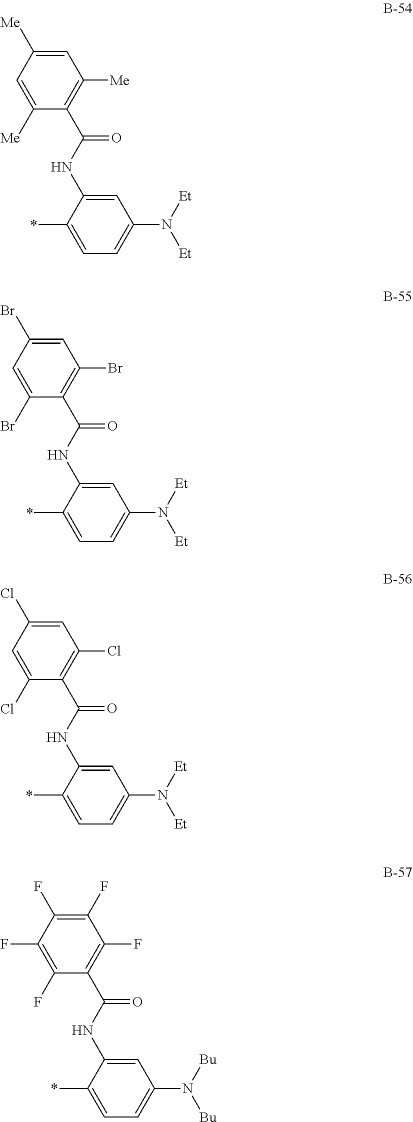

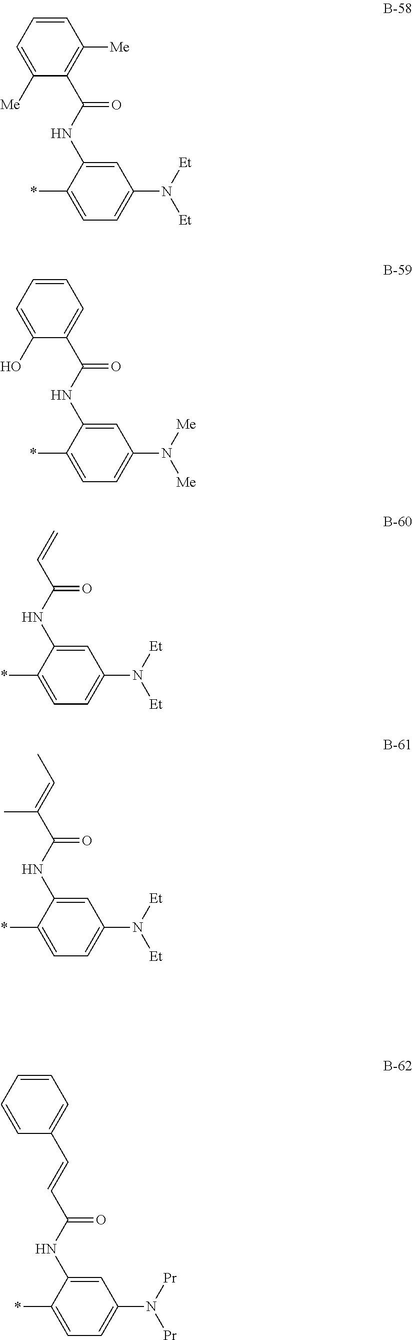

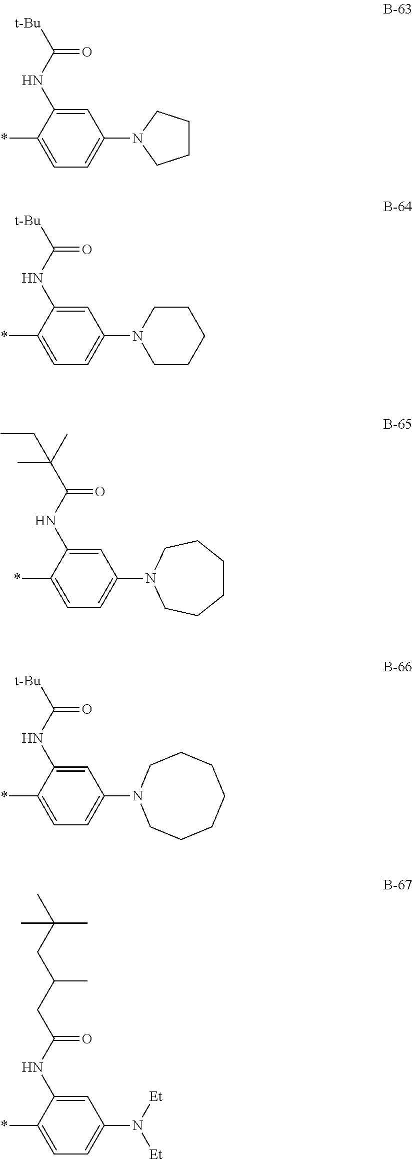

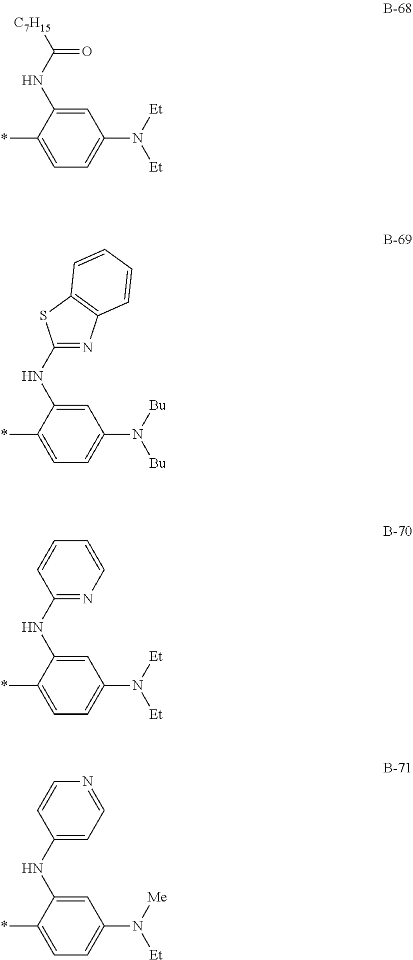

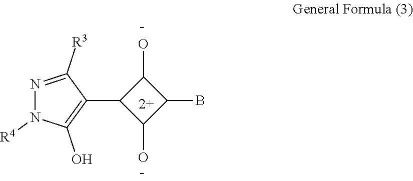

| Compound | | | |
|---|---|---|---|
| number | R³ | R⁴ | B |
| 3-1 | Me | Me | B-3 |
| 3-2 | Me | Me | B-4 |
| 3-3 | Me | Me | B-5 |
| 3-4 | Me | Me | B-10 |
| 3-5 | Me | Me | B-14 |
| 3-6 | Me | Me | B-16 |
| 3-7 | Me | Me | B-17 |
| 3-8 | Me | Me | B-18 |
| 3-9 | Me | Me | B-19 |
| 3-10 | Me | Me | B-20 |
| 3-11 | Me | Me | B-21 |
| 3-12 | Me | Me | B-22 |
| 3-13 | Me | Me | B-23 |
| 3-14 | Me | Me | B-26 |
| 3-15 | Me | Me | B-32 |
| 3-16 | Me | Me | B-33 |
| 3-17 | Me | Me | B-38 |
| 3-18 | Me | Me | B-49 |
| 3-19 | Et | 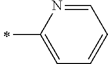 | B-28 |
| 3-20 | Me | 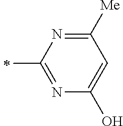 | B-29 |
| 3-21 | H | H | B-23 |
| 3-22 | Et | t-Bu | B-21 |
| 3-23 | t-Bu | Me | B-18 |
| 3-24 | CF₃ | i-Pr | B-12 |
| 3-25 | COOEt | Et | B-6 |
| 3-26 | CN | Ph | B-11 |
| 3-27 | NMe₂ | Me | B-2 |
| 3-28 | i-Pr | Me | B-17 |
| 3-29 | OEt | Bu | B-27 |
| 3-30 | NH₂ | i-Pr | B-9 |
| 3-31 | t-Bu | Me | B-17 |
| 3-32 | t-Bu | Bu | B-21 |
| 3-33 | CF₃ | Me | B-18 |
| 3-34 | OEt | Et | B-33 |
| 3-35 | NMe₂ | i-Pr | B-2 |
| 3-36 | Et | Me | B-17 |
| 3-37 | Bu | Me | B-18 |
| 3-38 | NH₂ | Ph | B-19 |
| 3-39 | OEt | 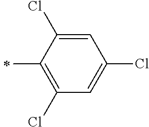 | B-25 |
| 3-40 | Me | 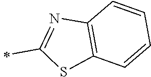 | B-2 |
| 3-41 | Me | Ph | B-17 |
| 3-42 | Me | Ph | B-21 |
| 3-43 | Me | Ph | B-36 |
| 3-44 | Me | t-Bu | B-17 |
| 3-45 | Me | t-Bu | B-18 |
| 3-46 | Me | t-Bu | B-10 |
| 3-47 | OEt | Me | B-17 |
| 3-48 | OEt | Me | B-10 |
| 3-49 | Me | 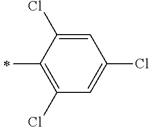 | B-17 |
| 3-50 | Me | 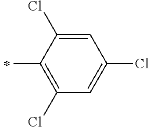 | B-19 |
| 3-51 | Me | 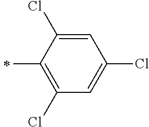 | B-21 |
| 3-52 | Me | 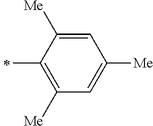 | B-17 |
| 3-53 | Me | 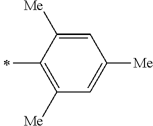 | B-20 |
| 3-54 | Me | 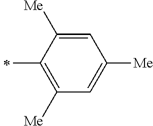 | B-21 |
| 3-55 | t-Bu | Me | B-17 |
| 3-56 | t-Bu | Me | B-10 |
| 3-57 | t-Bu | Me | B-44 |
| 3-58 | t-Bu | t-Bu | B-17 |
| 3-59 | t-Bu | t-Bu | B-10 |
| 3-60 | t-Bu | t-Bu | B-6 |
| 3-61 | NBu₂ | Me | B-17 |
| 3-62 | NBu₂ | Me | B-10 |
| 3-63 | t-Bu | 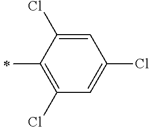 | B-17 |
| 3-64 | t-Bu | 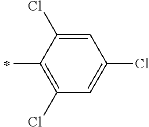 | B-19 |
| 3-65 | t-Bu | 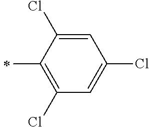 | B-21 |
| 3-66 | t-Bu | 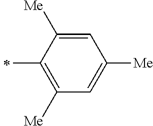 | B-17 |
| 3-67 | t-Bu | 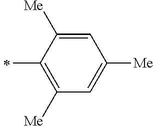 | B-20 |
| 3-68 | t-Bu | 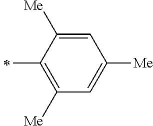 | B-21 |
| 3-69 | Me | t-Bu | B-51 |
| 3-70 | Me | t-Bu | B-52 |
| 3-71 | Me | t-Bu | B-54 |
| 3-72 | Me | t-Bu | B-55 |
| 3-73 | Me | t-Bu | B-58 |
| 3-74 | Me | t-Bu | B-60 |
| 3-75 | Me | t-Bu | B-65 |
| 3-76 | Me | t-Bu | B-67 |
| 3-77 | Me | t-Bu | B-68 |
| 3-78 | H | t-Bu | B-51 |
| 3-79 | Et | t-Bu | B-53 |
| 3-80 | Pr | 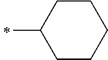 | B-64 |
| 3-81 | iPr | iPr | B-66 |
| 3-82 | Me | 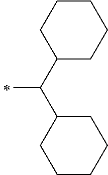 | B-51 |
| 3-83 | Et | Bu | B-56 |
| 3-84 | Me | iPr | B-66 |
| 3-85 | Me | 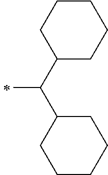 | B-54 |
| 3-86 | Me | 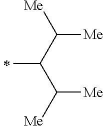 | B-57 |
| 3-87 | Et | 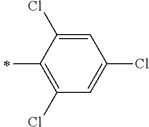 | B-60 |
| 3-88 | Me | iPr | B-65 |
| 3-89 | Me | t-Bu | B-69 |
| 3-90 | Me | 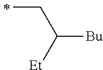 | B-50 |
| 3-91 | Me | 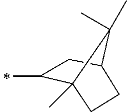 | B-61 |
| 3-92 | Me | 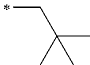 | B-51 |
| 3-93 | Me | 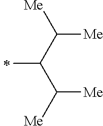 | B-51 |
| 3-94 | Me | 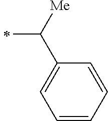 | B-67 |
| 3-95 | Me | 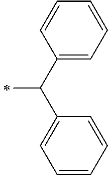 | B-51 |
| 3-96 | Me | 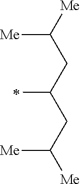 | B-51 |

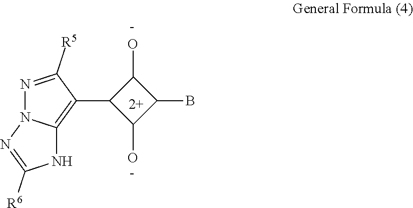

| Compound | | | |
|---|---|---|---|
| number | R⁵ | R⁶ | B |
| 4-1 | t-Bu | 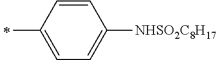 | B-2 |
| 4-2 | t-Bu | 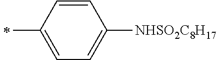 | B-6 |
| 4-3 | t-Bu | 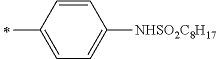 | B-10 |
| 4-4 | Me | 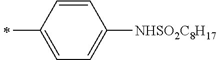 | B-4 |
| 4-5 | t-Bu | 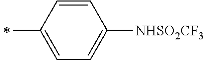 | B-6 |
| 4-6 | t-Bu | 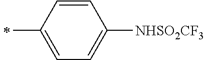 | B-14 |
| 4-7 | NHCOCH₃ | 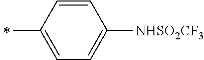 | B-1 |
| 4-8 | t-Bu | 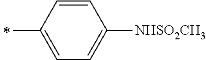 | B-6 |
| 4-9 | t-Bu | 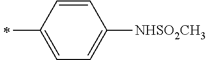 | B-16 |
| 4-10 | OEt | 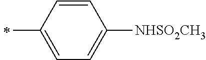 | B-11 |
| 4-11 | t-Bu | 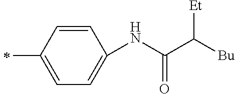 | B-6 |
| 4-12 | t-Bu | 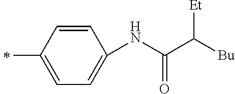 | B-12 |
| 4-13 | OEt | 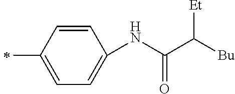 | B-31 |
| 4-14 | H | H | B-22 |
| 4-15 | Me | Me | B-23 |
| 4-16 | Me | Me | B-17 |
| 4-17 | Me | Et | B-18 |
| 4-18 | Ph | Ph | B-8 |
| 4-19 | Et | t-Bu | B-17 |
| 4-20 | OEt | t-Bu | B-3 |
| 4-21 | OEt | Bu | B-26 |
| 4-22 | OEt | 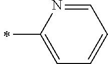 | B-2 |
| 4-23 | CF3 | t-Bu | B-19 |
| 4-24 | NHCOCH₃ | t-Bu | B-2 |
| 4-25 | NHCOCH₃ | Me | B-1 |
| 4-26 | NMe₂ | t-Bu | B-6 |
| 4-27 | NMe₂ | Et | B-17 |
| 4-28 | H | Me | B-2 |
| 4-29 | t-Bu | t-Bu | B-18 |
| 4-30 | t-Bu | Me | B-17 |
| 4-31 | t-Bu | 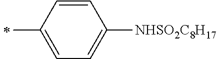 | B-51 |
| 4-32 | t-Bu | 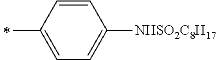 | B-52 |
| 4-33 | t-Bu | 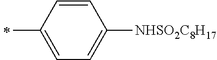 | B-54 |
| 4-34 | Me | 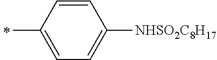 | B-55 |
| 4-35 | t-Bu | 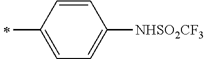 | B-60 |
| 4-36 | Me | Me | B-65 |
| 4-37 | Me | Et | B-67 |
| 4-38 | Ph | Ph | B-48 |
| 4-39 | Et | t-Bu | B-54 |
| 4-40 | Me | Me | B-51 |

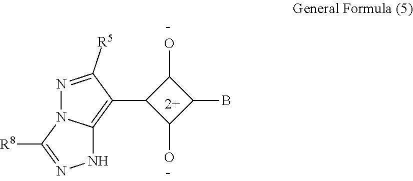

| Compound | | | |
|---|---|---|---|
| number | R⁷ | R⁸ | B |
| 5-1 | t-Bu | 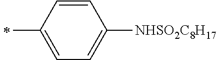 | B-2 |
| 5-2 | Me | 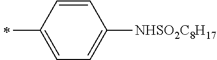 | B-6 |
| 5-3 | t-Bu | 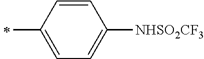 | B-4 |
| 5-4 | Me | 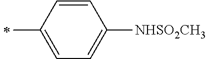 | B-10 |
| 5-5 | t-Bu | 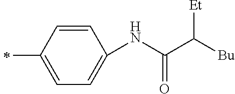 | B-6 |
| 5-6 | t-Bu | 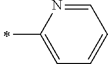 | B-14 |
| 5-7 | Me | 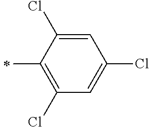 | B-1 |
| 5-8 | Me | 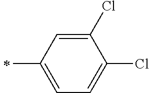 | B-6 |
| 5-9 | Me | 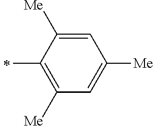 | B-16 |
| 5-10 | t-Bu | 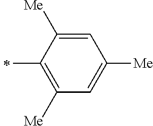 | B-11 |
| 5-11 | Me | Me | B-17 |
| 5-12 | Me | t-Bu | B-18 |
| 5-13 | Ph | Ph | B-8 |
| 5-14 | Ph | 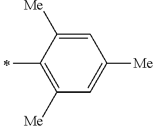 | B-17 |
| 5-15 | Et | Ph | B-17 |
| 5-16 | OEt | t-Bu | B-3 |
| 5-17 | OEt | Bu | B-26 |
| 5-18 | CF3 | t-Bu | B-19 |
| 5-19 | NHCOCH3 | t-Bu | B-2 |
| 5-20 | NHCOCH3 | 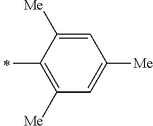 | B-1 |
| 5-21 | t-Bu | 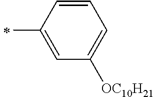 | B-2 |
| 5-22 | Me | 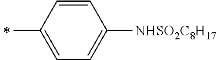 | B-51 |
| 5-23 | t-Bu | 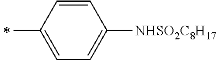 | B-52 |
| 5-24 | Me | 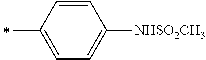 | B-55 |
| 5-25 | t-Bu | 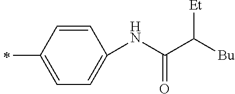 | B-60 |
| 5-26 | Me | Me | B-65 |
| 5-27 | Me | t-Bu | B-67 |
| 5-28 | Ph | Ph | B-50 |
| 5-29 | Ph | 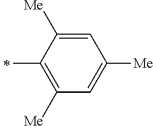 | B-23 |
| 5-30 | Et | Ph | B-59 |

For the preferred embodiment of the coloring agent represented by General Formula (1), a coloring agent represented by any of General Formulae (6) to (9) described in [0081] to [0095] of WO2021/132674A and the description regarding the specific example can be applied as they are.

(Quencher-Embedded Coloring Agent)

The squaraine-based coloring agent represented by General Formula (1) may be a quencher-embedded coloring agent in which a quencher moiety is linked to a coloring agent by a covalent bond through a linking group. The quencher-embedded coloring agent can also be preferably used as the above dye. That is, the quencher-embedded coloring agent is counted as the above dye according to the wavelength having the main absorption wavelength band.

Examples of the quencher-embedded coloring agent include an electron-donating quencher-embedded coloring agent in which the quencher moiety is an electron-donating quencher moiety, and an electron-accepting quencher moiety in which the quencher moiety is an electron-accepting quencher moiety.

The electron-donating quencher moiety means a structure portion that inactivates a coloring agent in the excited state to the ground state by donating an electron to a SOMO at a low energy level of two SOMO's of the coloring agent in the excited state and then receiving an electron from a SOMO at a high energy level of the coloring agent. The electron-accepting quencher moiety means a structure portion that inactivates a coloring agent in the excited state to the ground state by accepting an electron from a SOMO at a high energy level of two SOMO's of the coloring agent in the excited state and then donating an electron to a SOMO at a low energy level of the coloring agent.

Examples of the electron-donating quencher moiety include the ferrocenyl group in the substituent X described above, and the quencher moieties in the quencher compounds described in paragraphs [0199] to [0212] and paragraphs [0234] to [0287] of WO2019/066043A, where the ferrocenyl group in the substituent X described above is preferable. In addition, examples of the electron-accepting quencher moiety include the quencher moieties in the quencher compounds described in paragraphs [0288] to [0310] of WO2019/066043A.

In a case where the light absorption filter according to the embodiment of the present invention contains a dye having a main absorption wavelength band in a wavelength range of 400 to 700 nm and a resin composed of a polymer including a partial structure that generates a radical upon ultraviolet irradiation, from the viewpoint of the light resistance of the light absorptive portion, the dye having a main absorption wavelength band in a wavelength range of 400 to 700 nm preferably includes an electron-donating quencher-embedded coloring agent, and more preferably includes a squaraine-based coloring agent represented by General Formula (1A) described below.

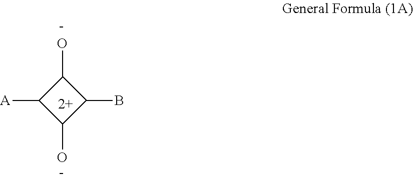

In the formula, A and B each independently represent an aryl group which may have a substituent, a heterocyclic group which may have a substituent, or —CH=G.

Here, G represents a heterocyclic group which may have a substituent. However, at least one of A or B includes an electron-donating quencher moiety.

The coloring agent represented by General Formula (1A) is the same as the coloring agent represented by (1) described later, except that in the coloring agent represented by General Formula (1) described above, at least one of A or B includes an electron-donating quencher moiety. As a result, the description related to A, B, and G in General Formula (1) can be applied to the description related to A, B, and G in General Formula (1A). In addition, as a preferred embodiment of the coloring agent represented by General Formula (1A), a description in which, in the description of the coloring agent represented by any of General Formulae (2) to (9), which is a preferred embodiment of the coloring agent represented General Formula (1), at least one of the structures corresponding to A and B in General Formula (1) is changed to include an electron-donating quencher moiety can be applied.

The electron-donating quencher moiety contained in at least one of A and B is preferably the ferrocenyl group in the substituent X described above.

Among the squaraine-based coloring agents represented by General Formula (1), specific examples of the coloring agent corresponding to the quencher-embedded coloring agent are shown below. However, the present invention is not limited thereto.

In the following specific examples, Me represents methyl, Et represents ethyl, and Bu represents butyl, respectively.

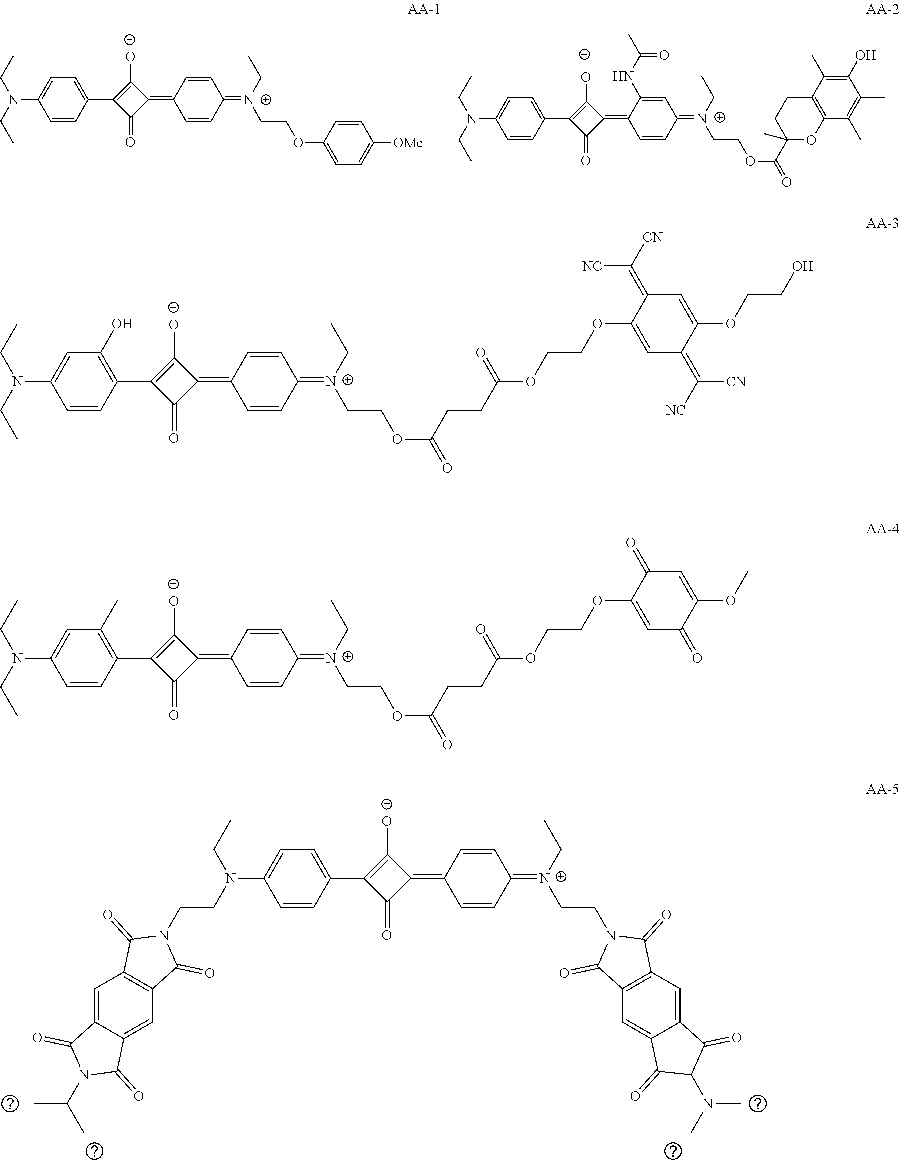

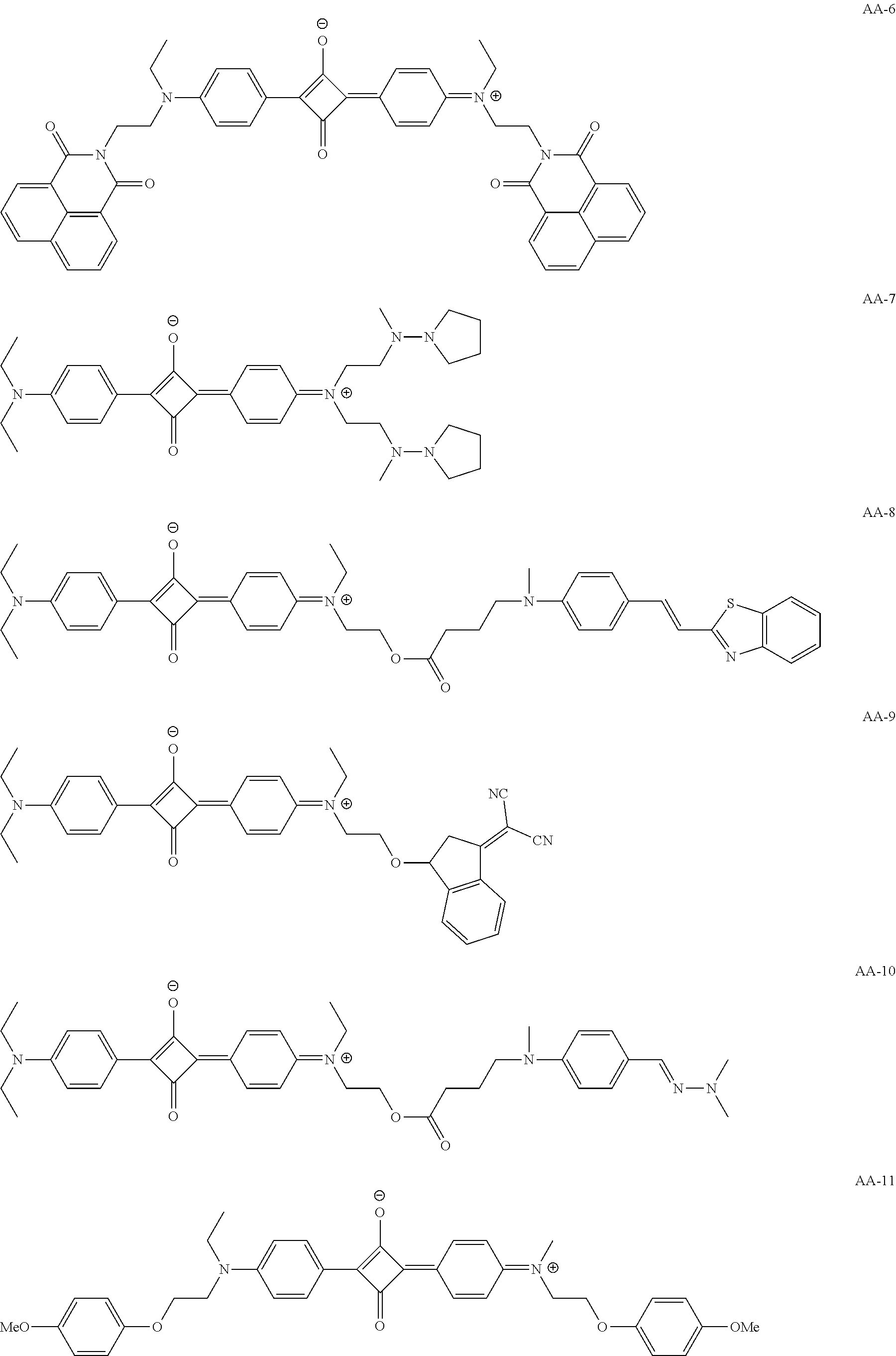

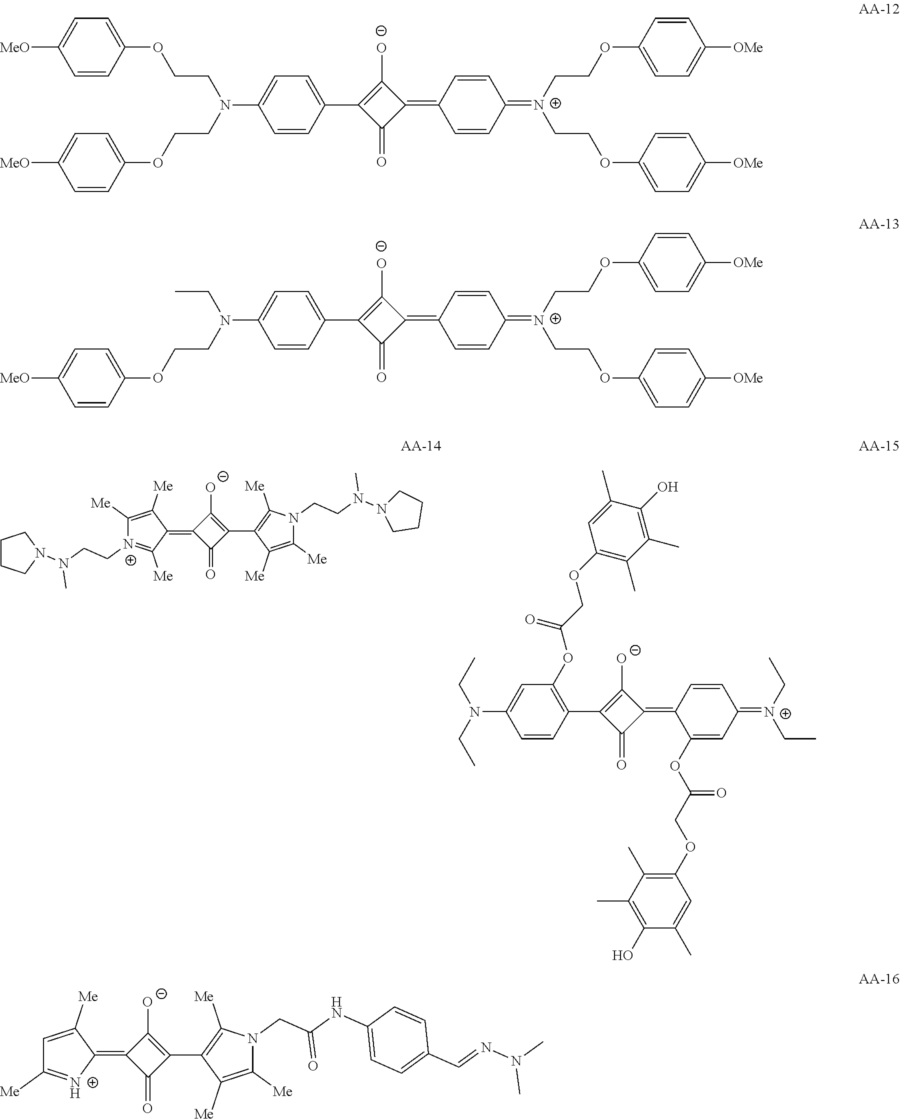

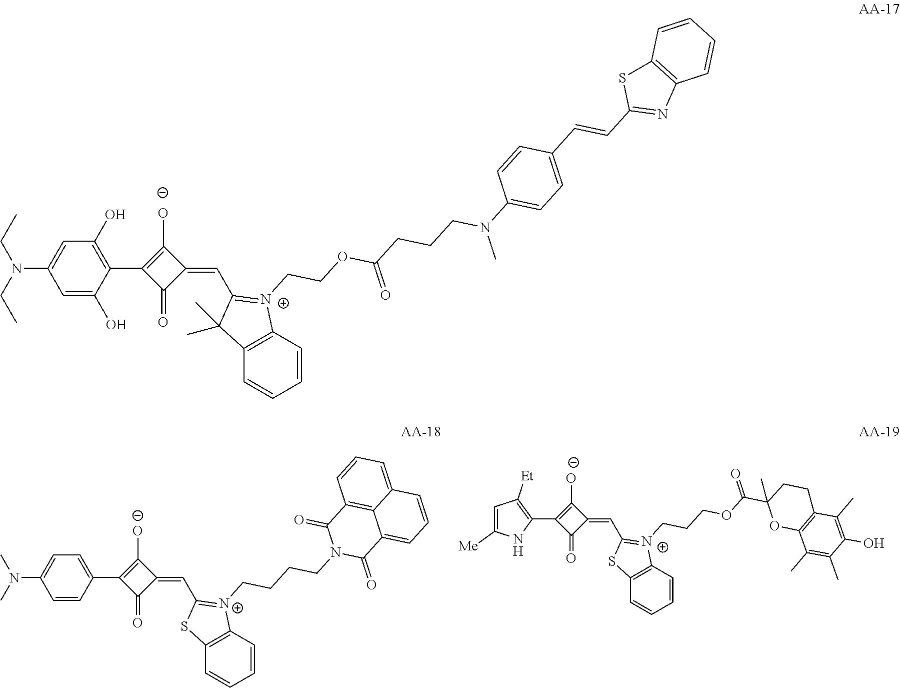 [SMILES: CCN(CC)c1cc(O)c(C2=C([O-])/C(=C/C3=[N+](CCOC(=O)CCCN(C)c4ccc(/C=C/c5nc6ccccc6s5)cc4)c4ccccc4C3(C)C)C2=O)c(O)c1 ; CN(C)c1ccc(C2=C([O-])/C(=C/c3sc4ccccc4[n+]3CCCCN3C(=O)c4cccc5cccc(c45)C3=O)C2=O)cc1 ; CCc1cc(C)[nH]c1C1=C([O-])/C(=C/c2sc3ccccc3[n+]2CCCOC(=O)C2(C)CCc3c(C)c(O)c(C)c(C)c3O2)C1=O]

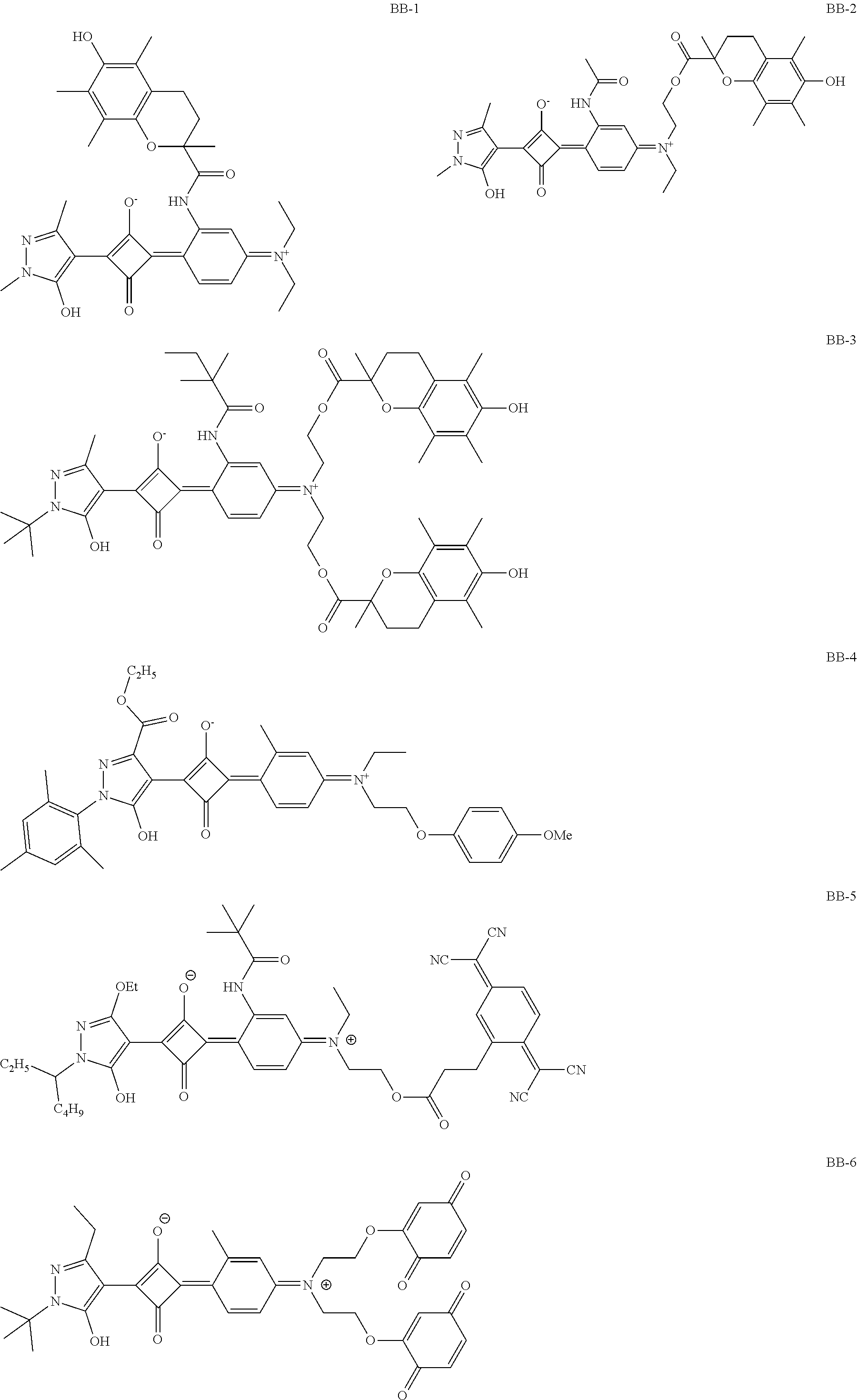

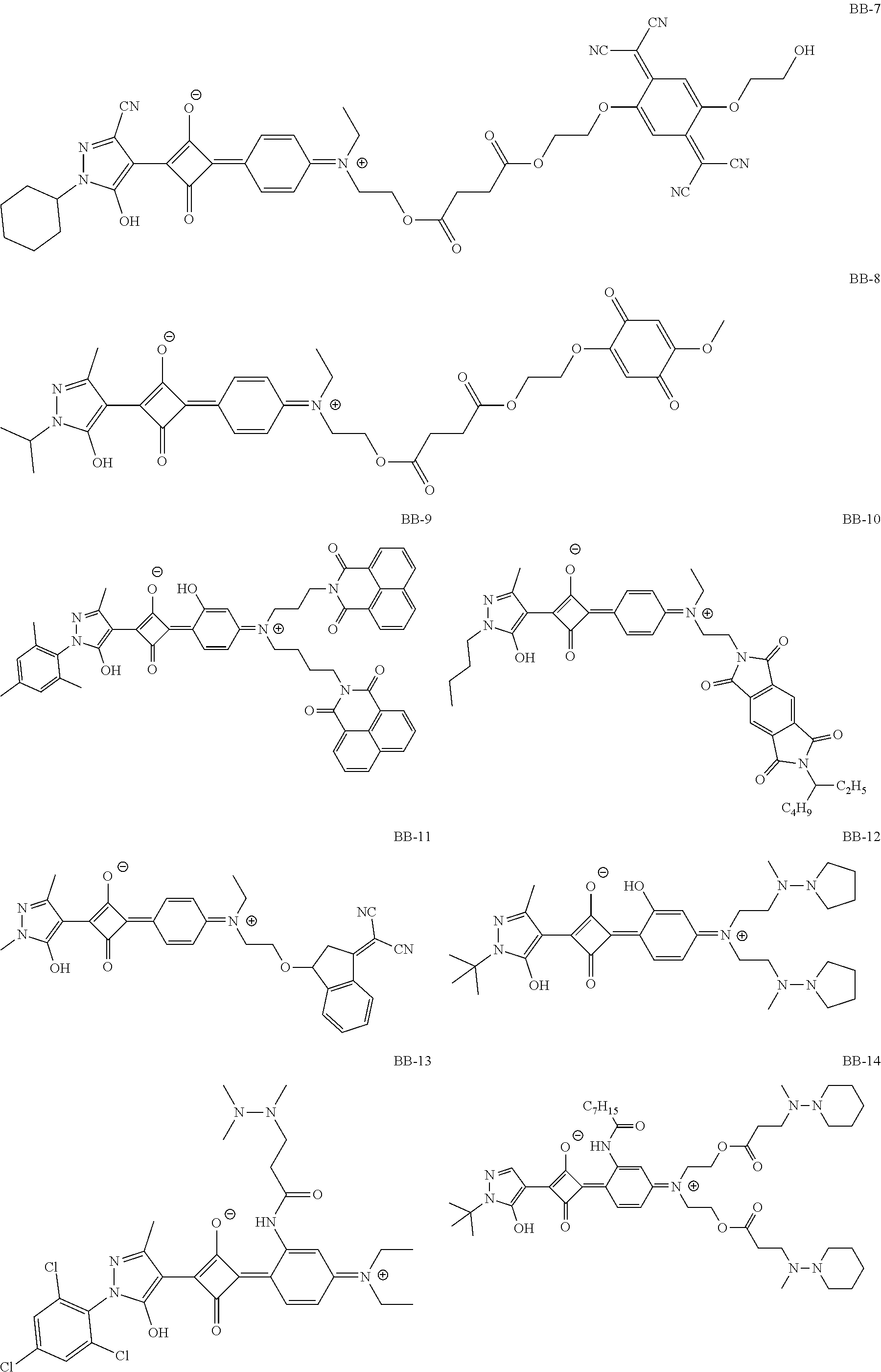

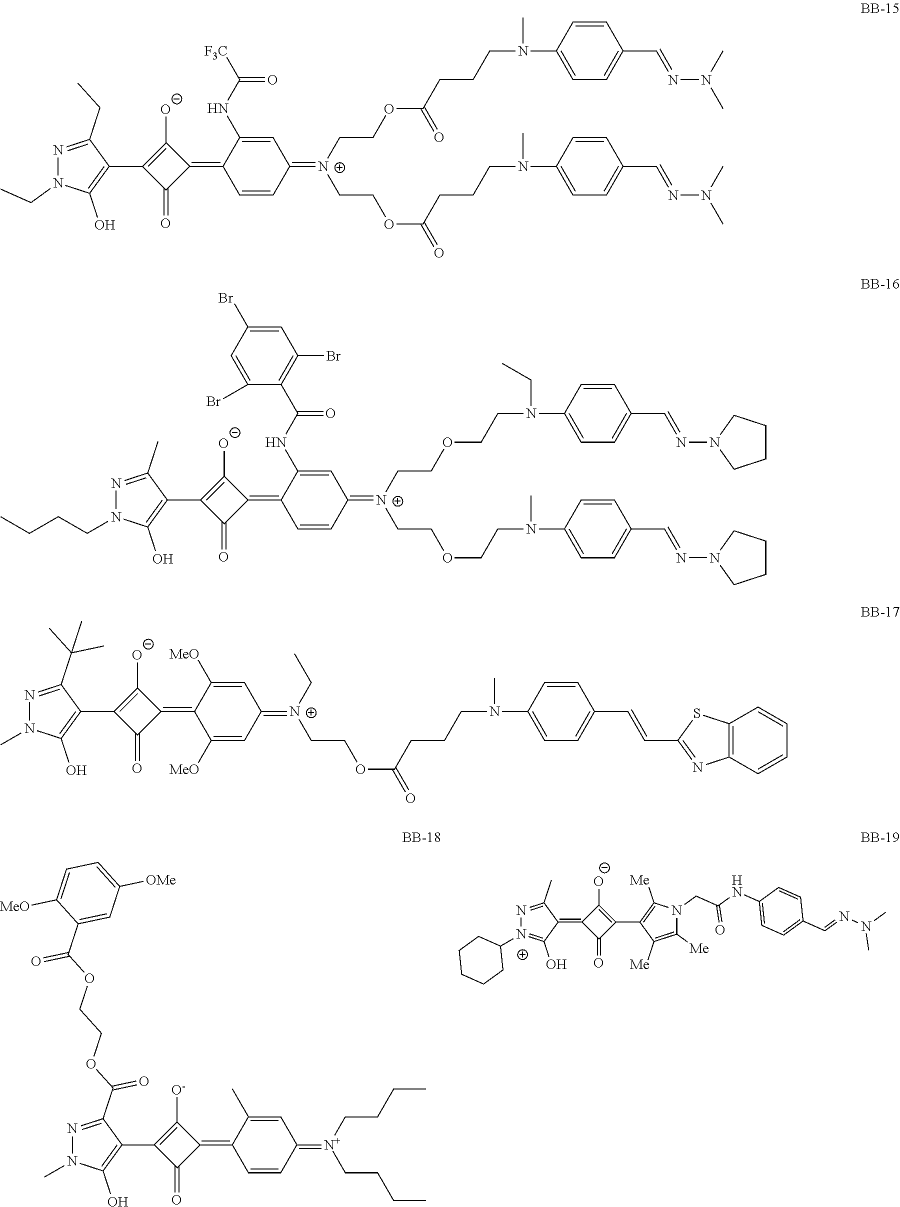

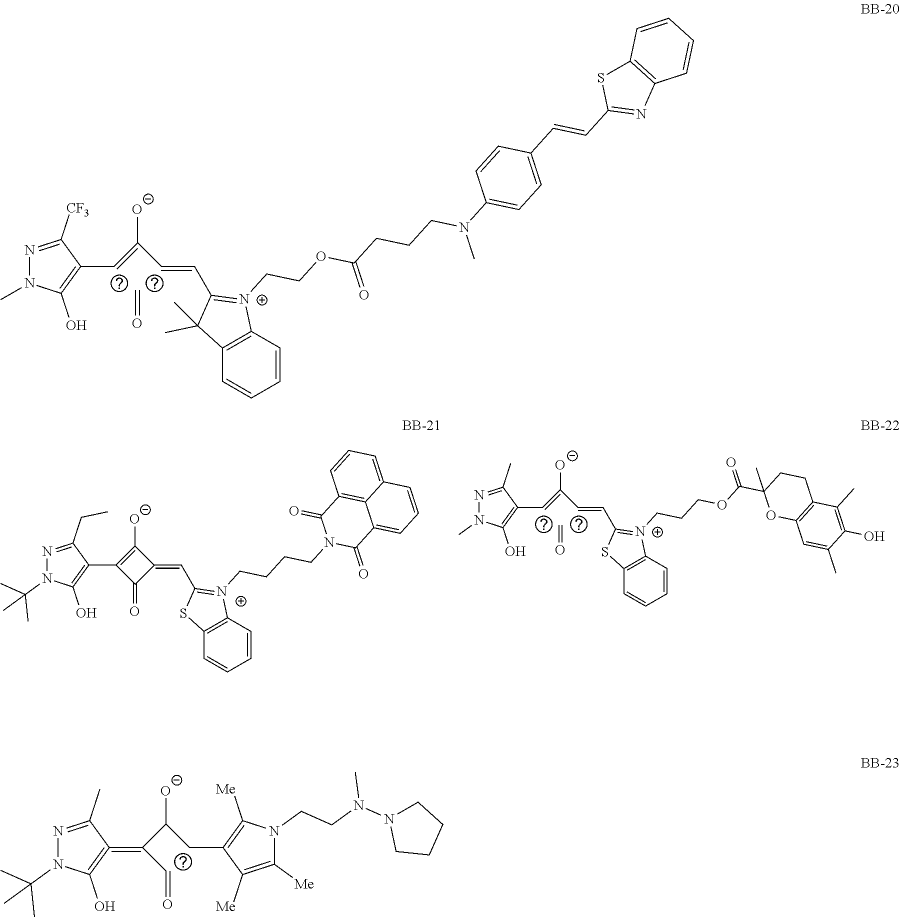

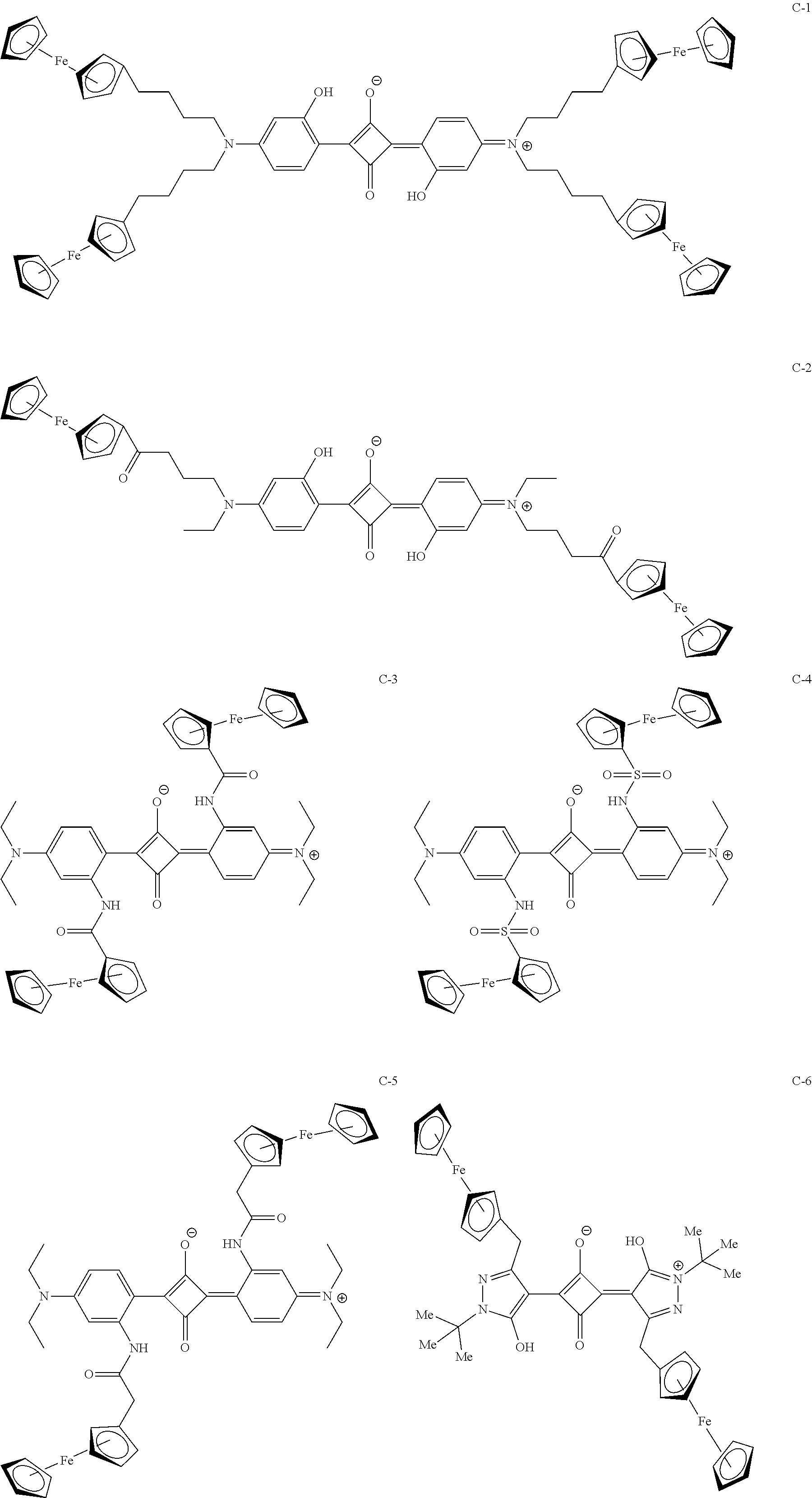

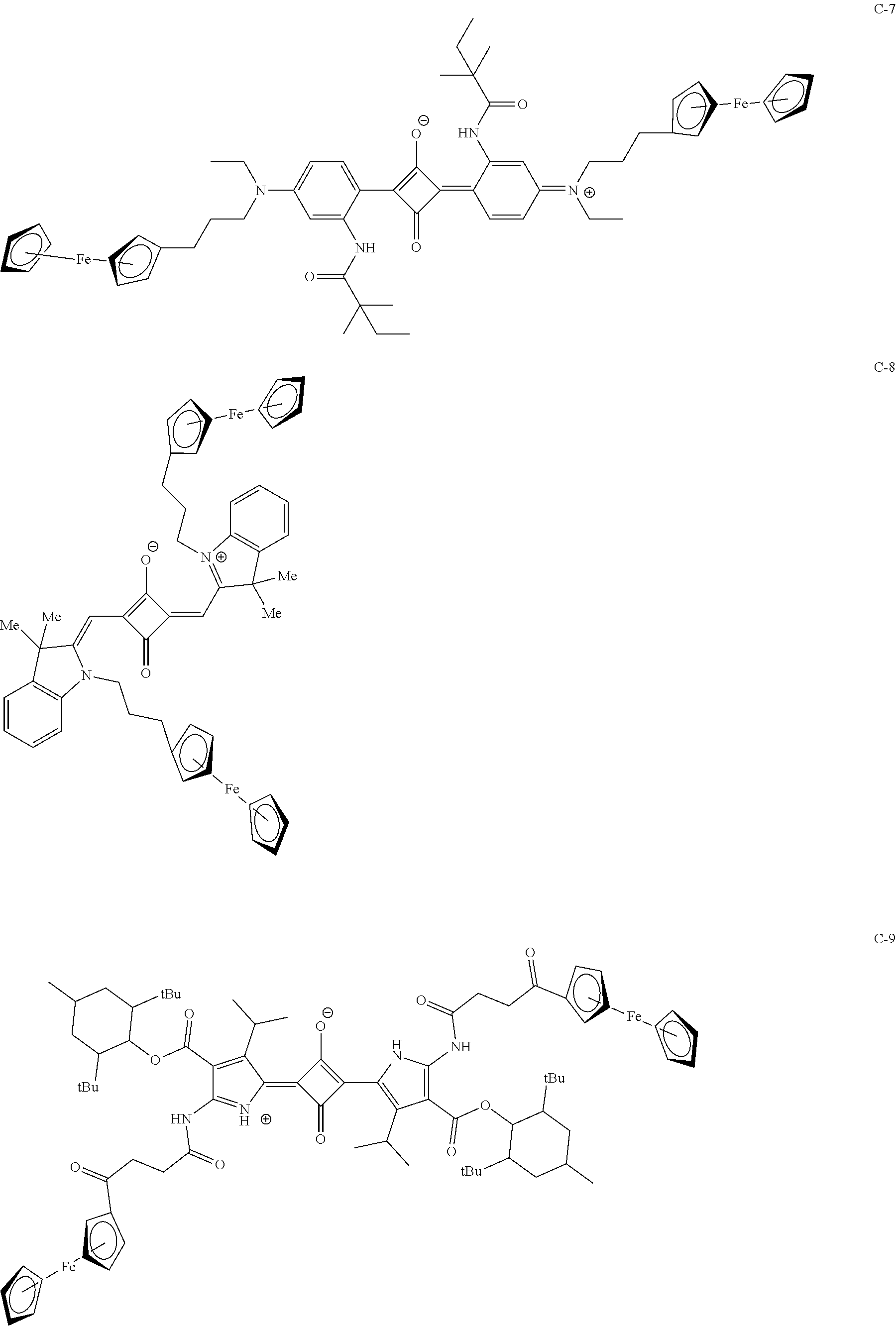

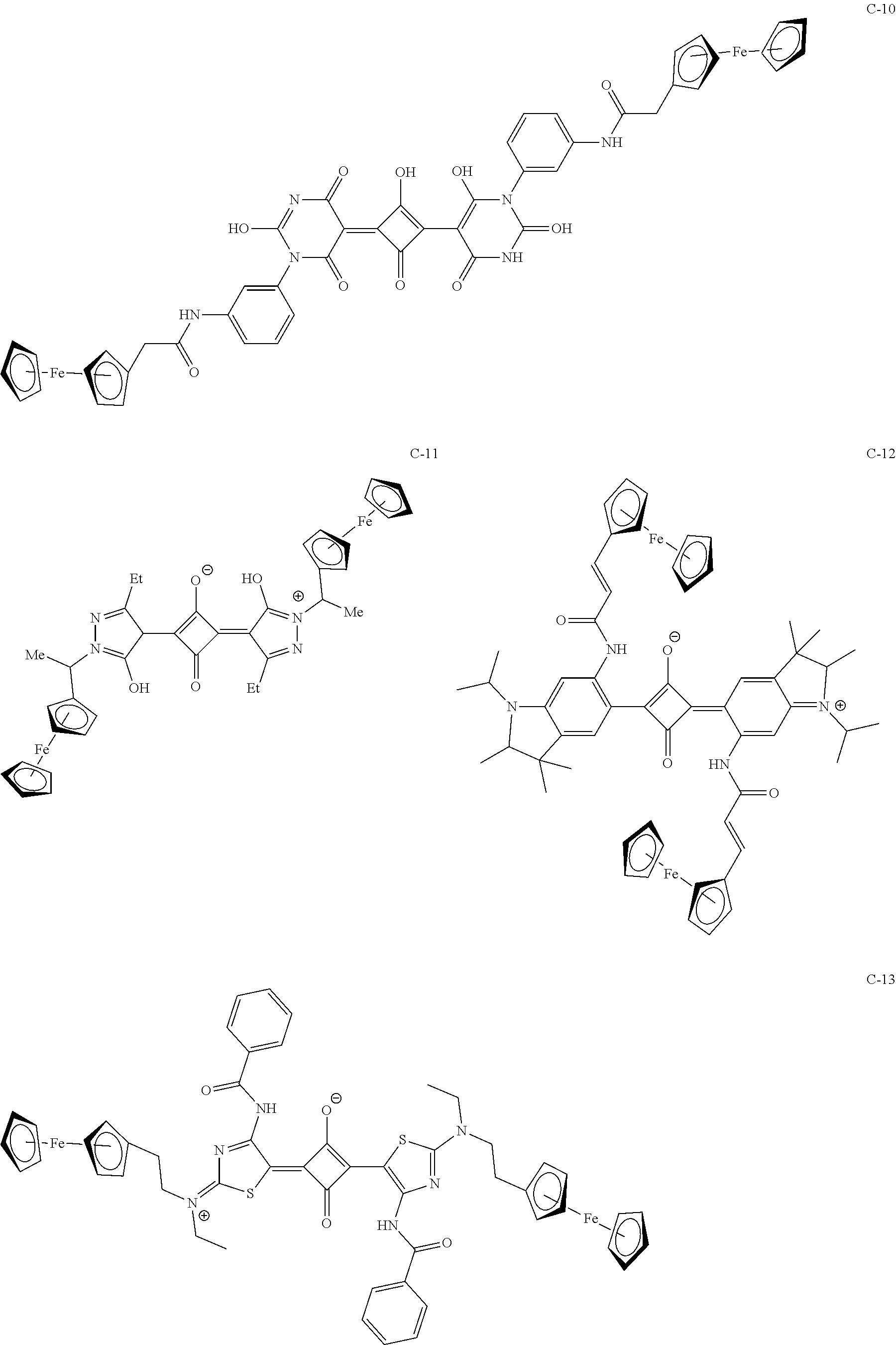

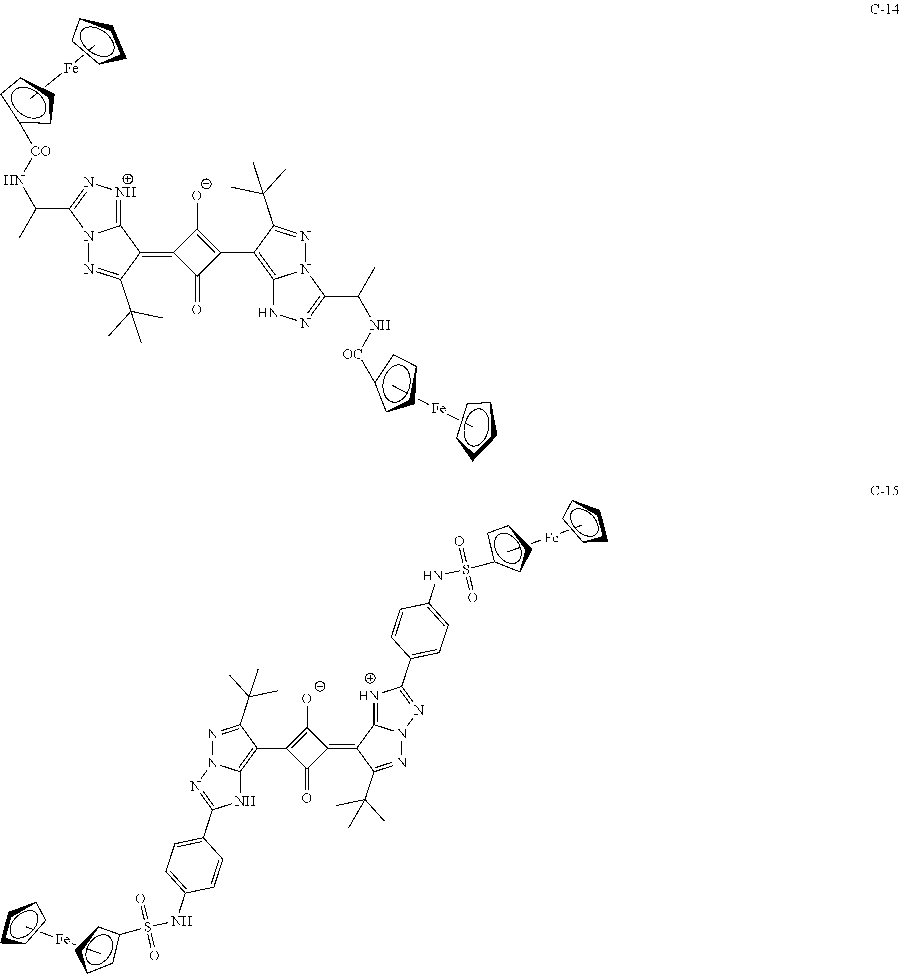

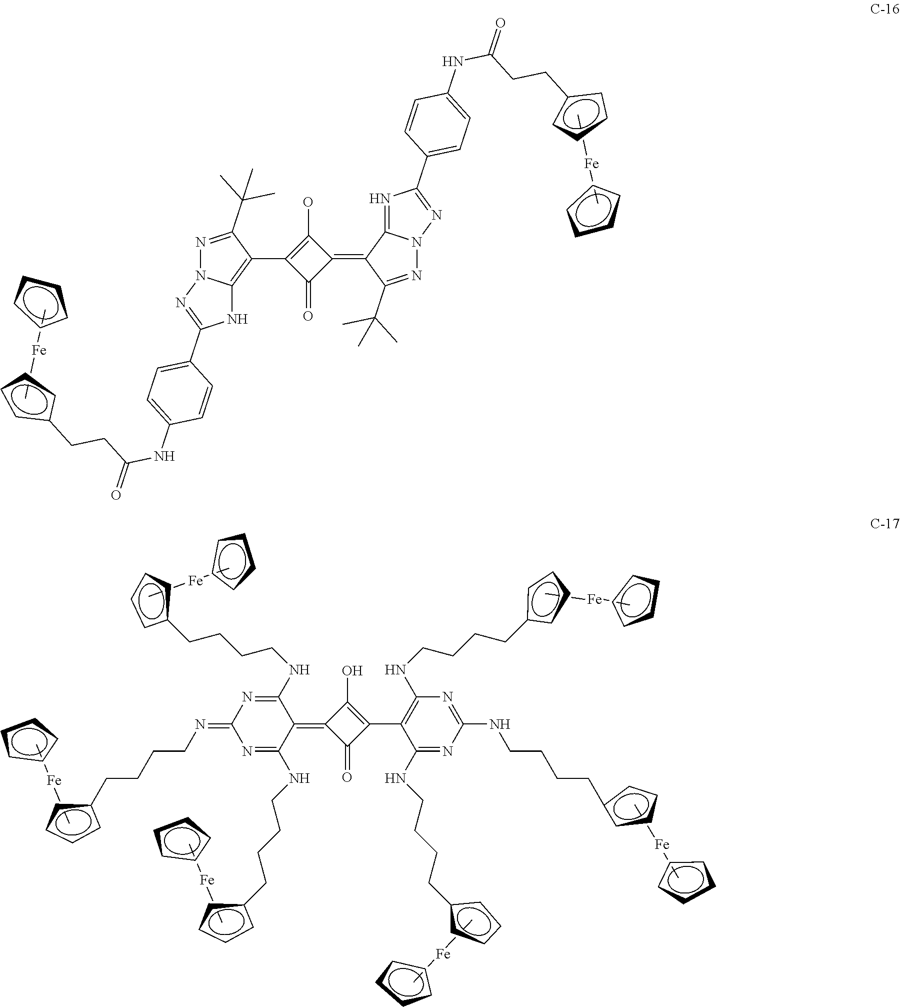

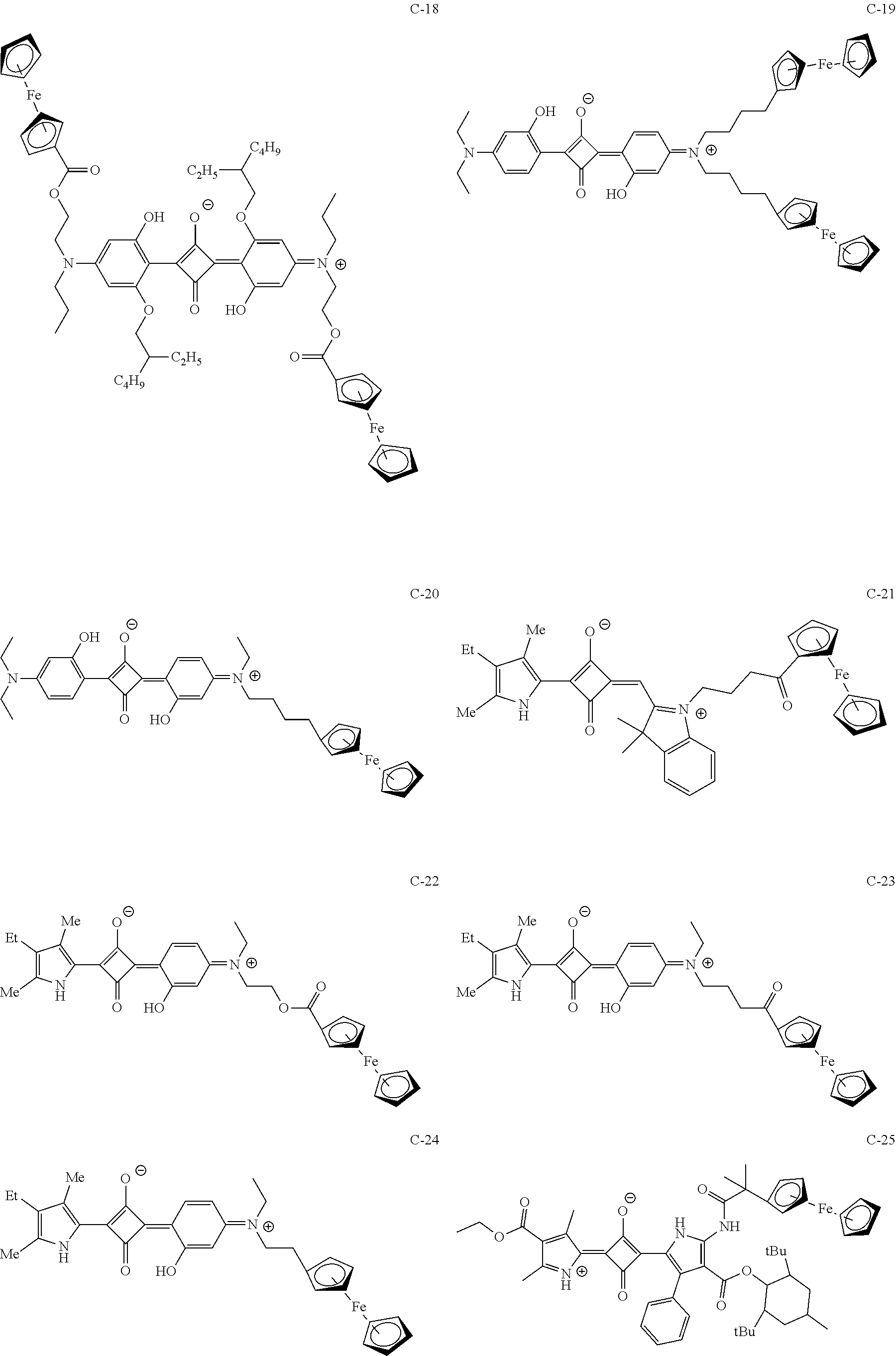

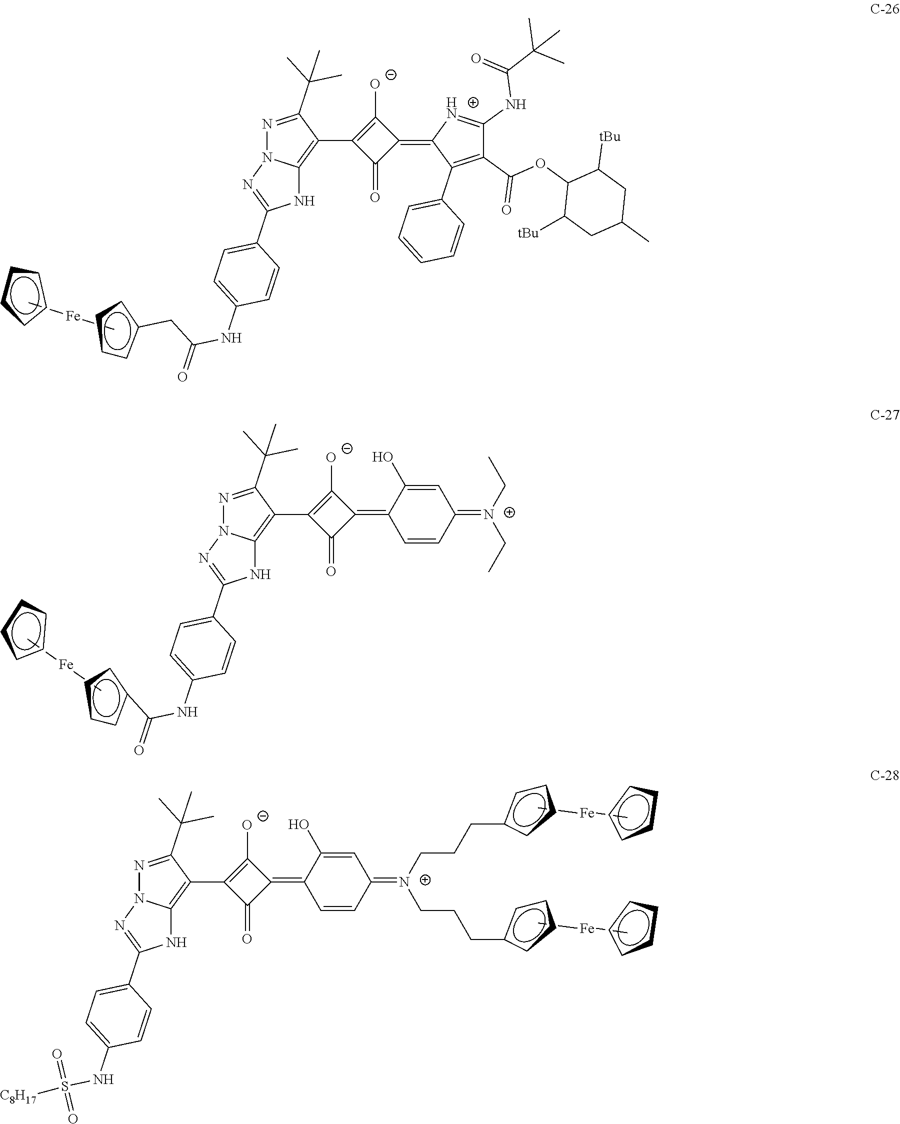

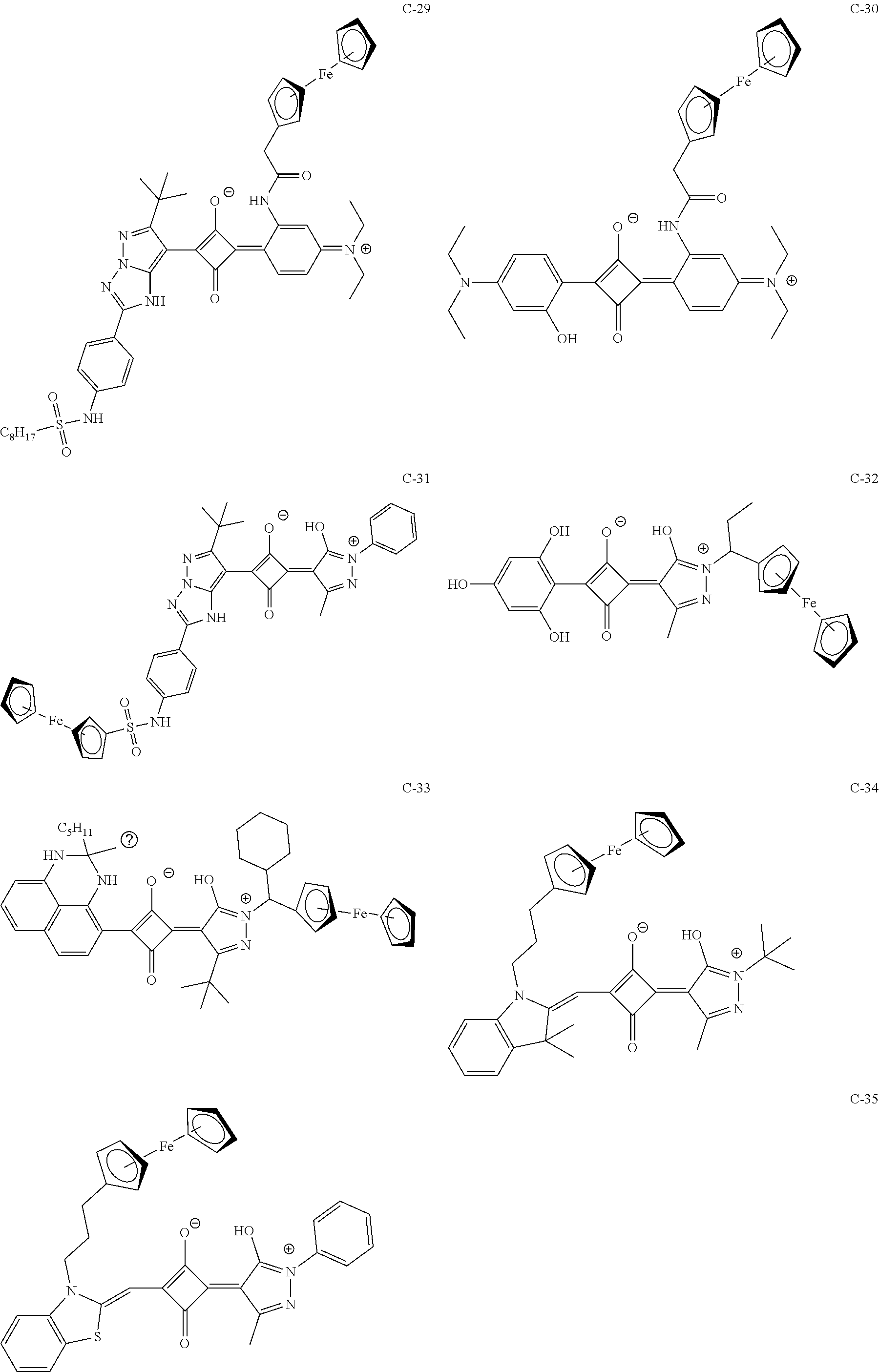

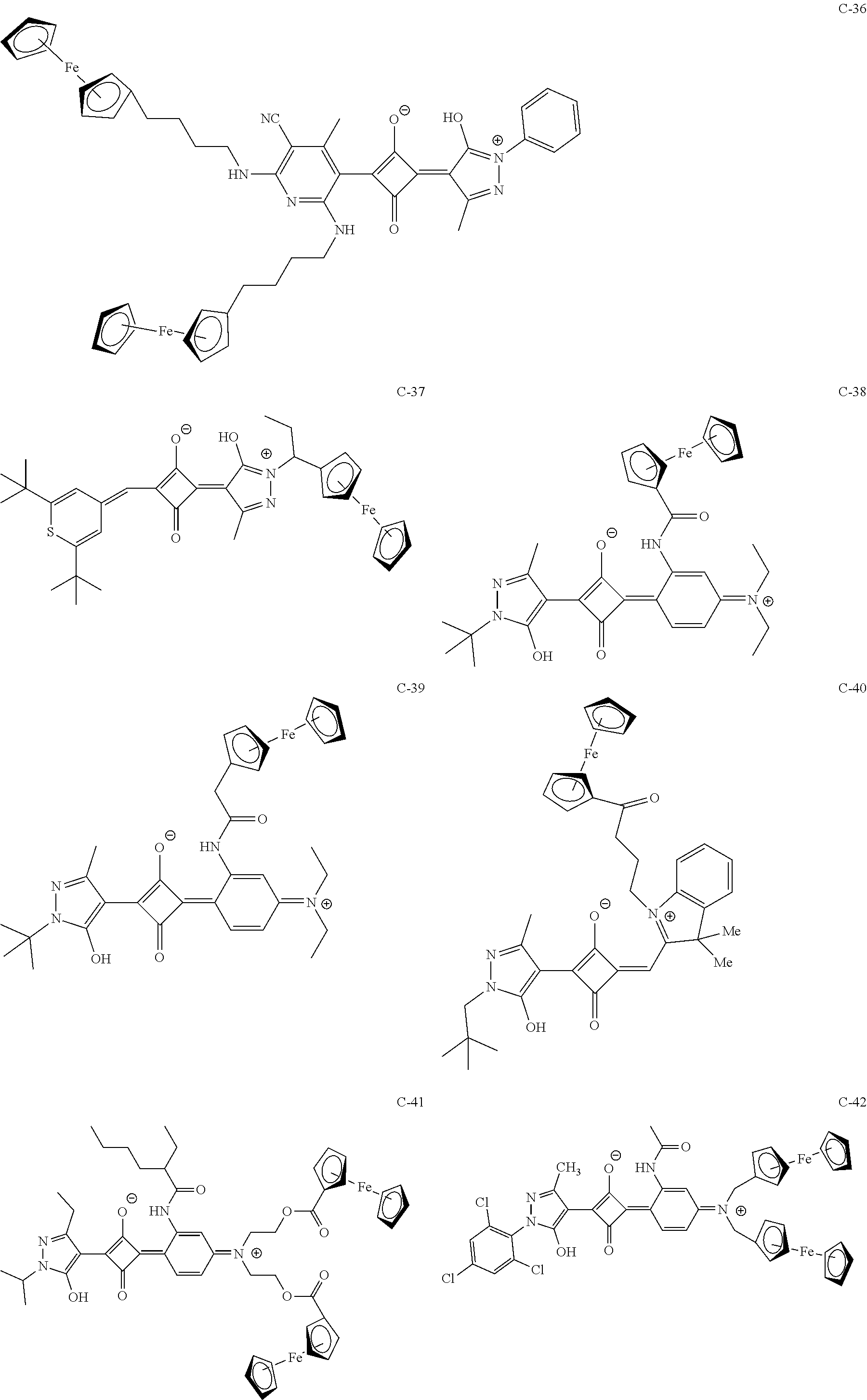

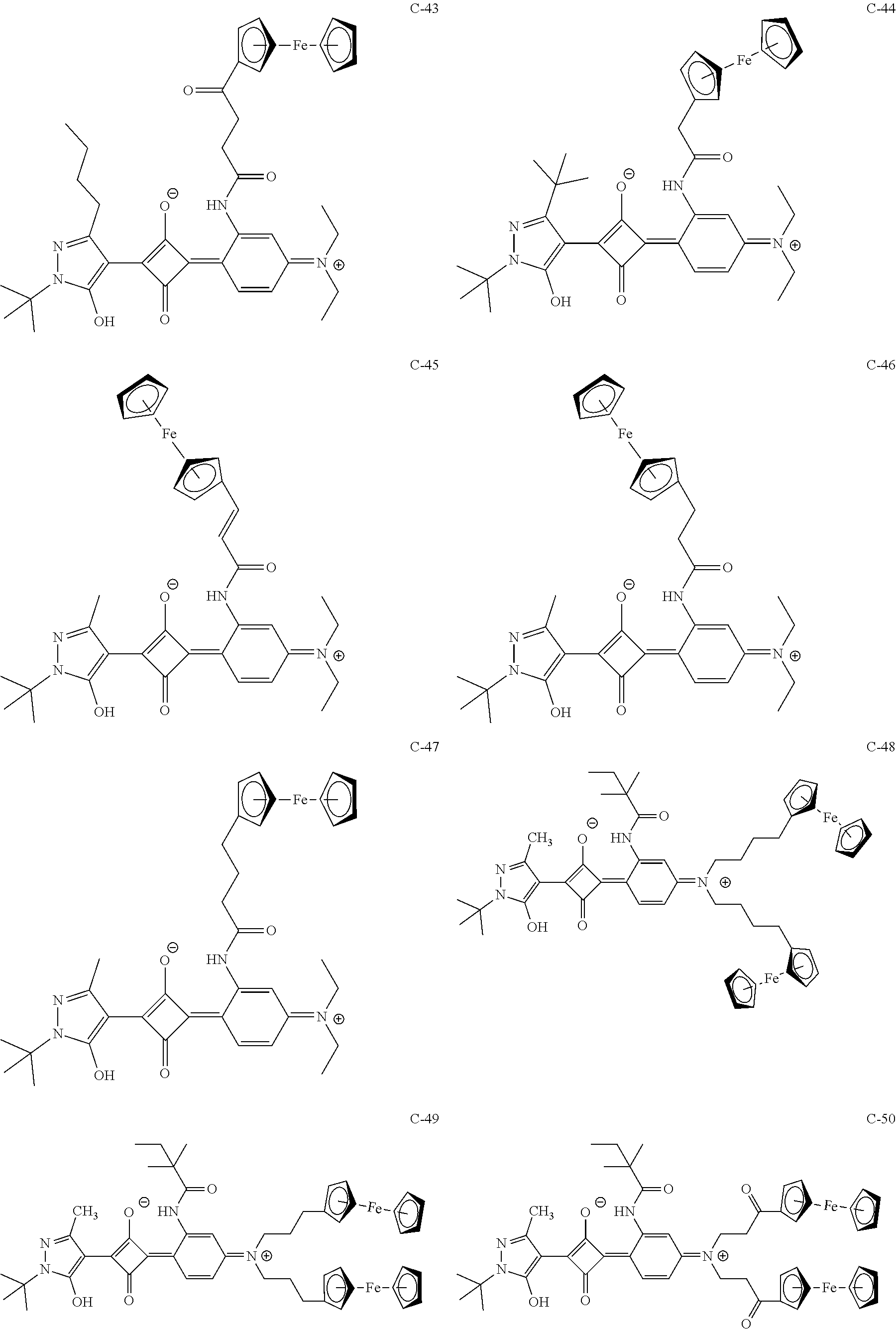

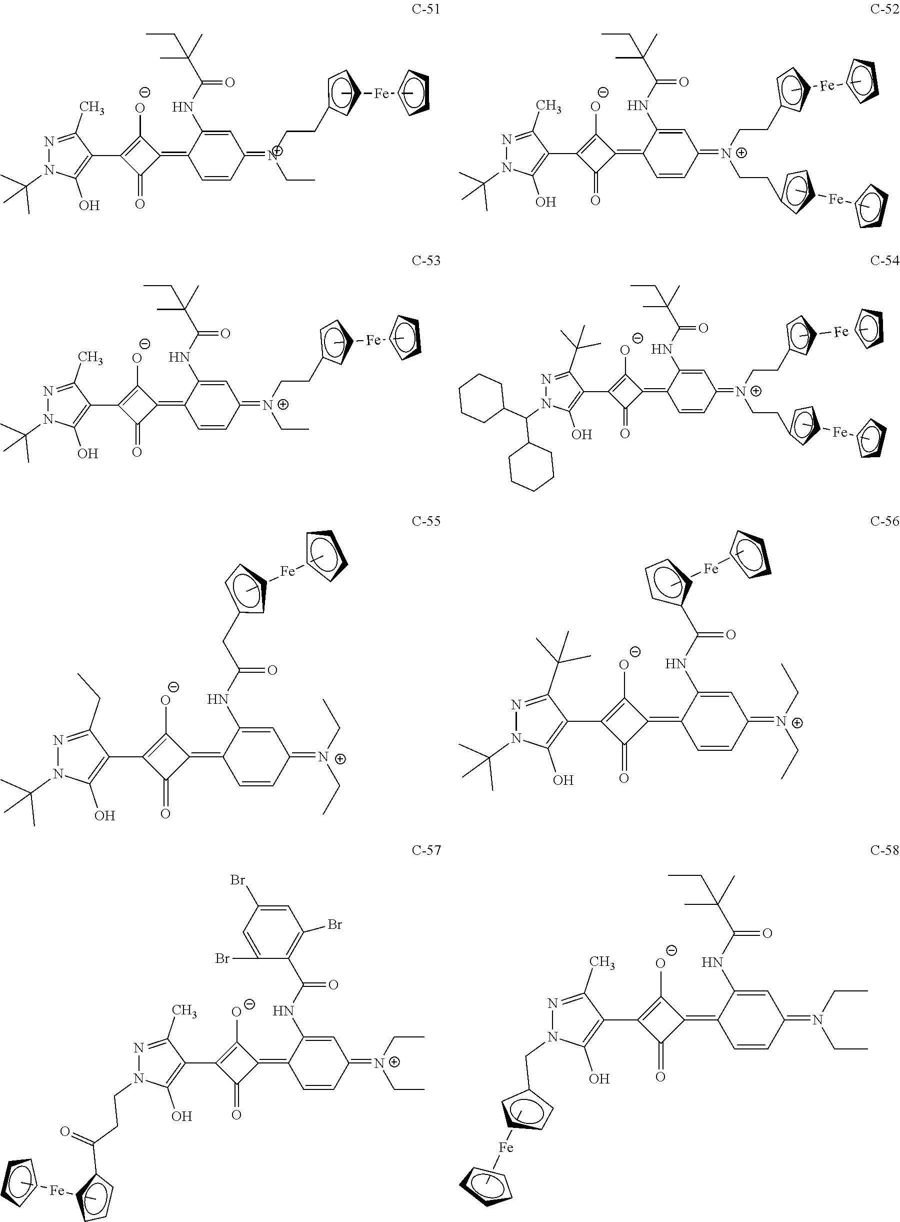

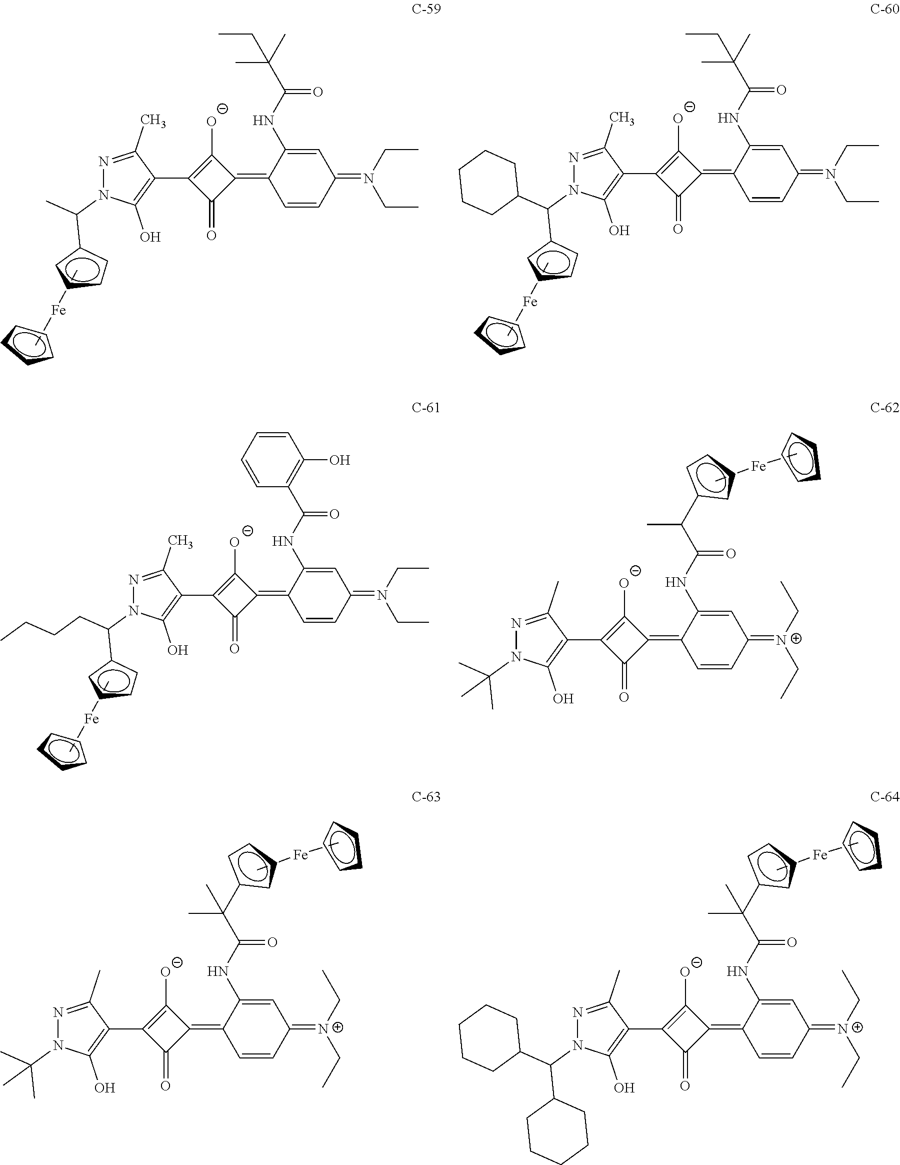

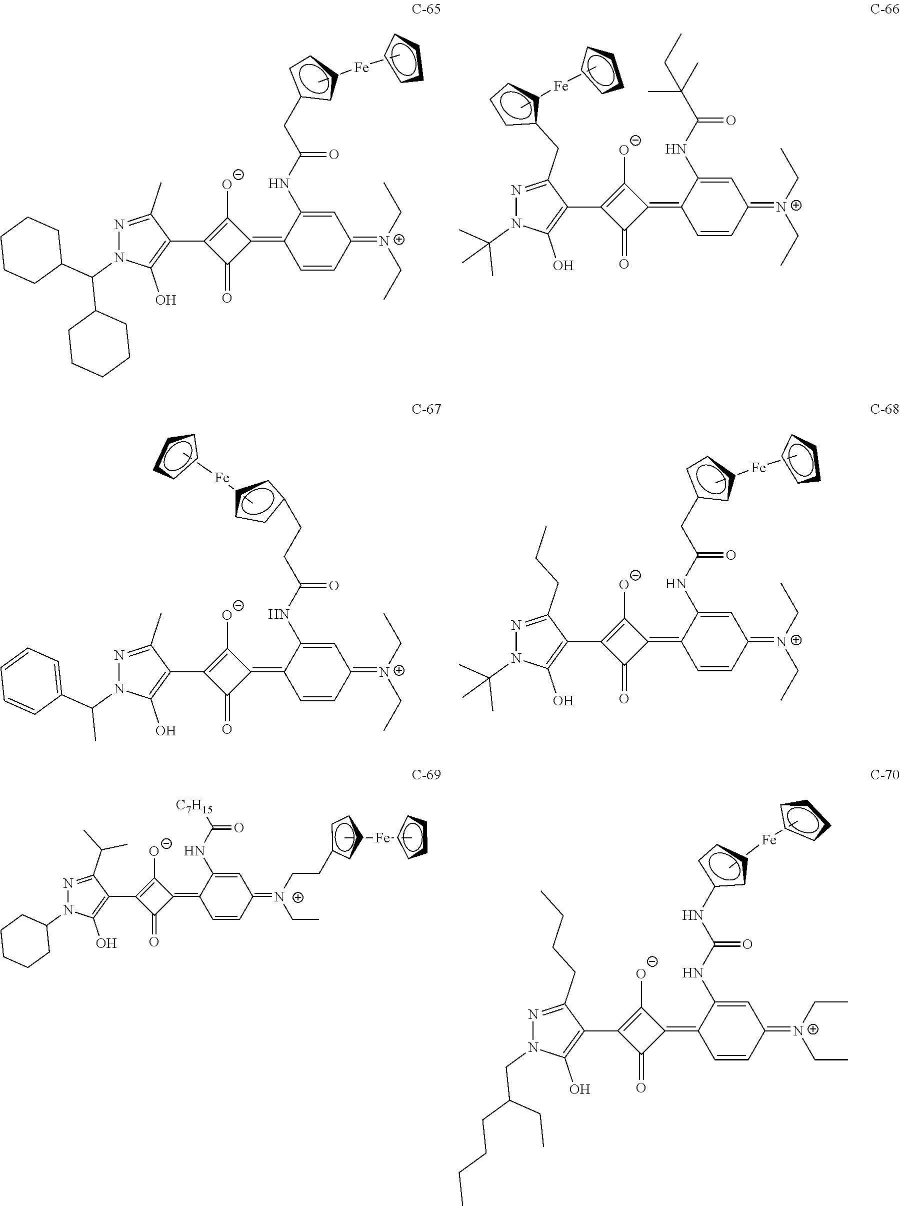

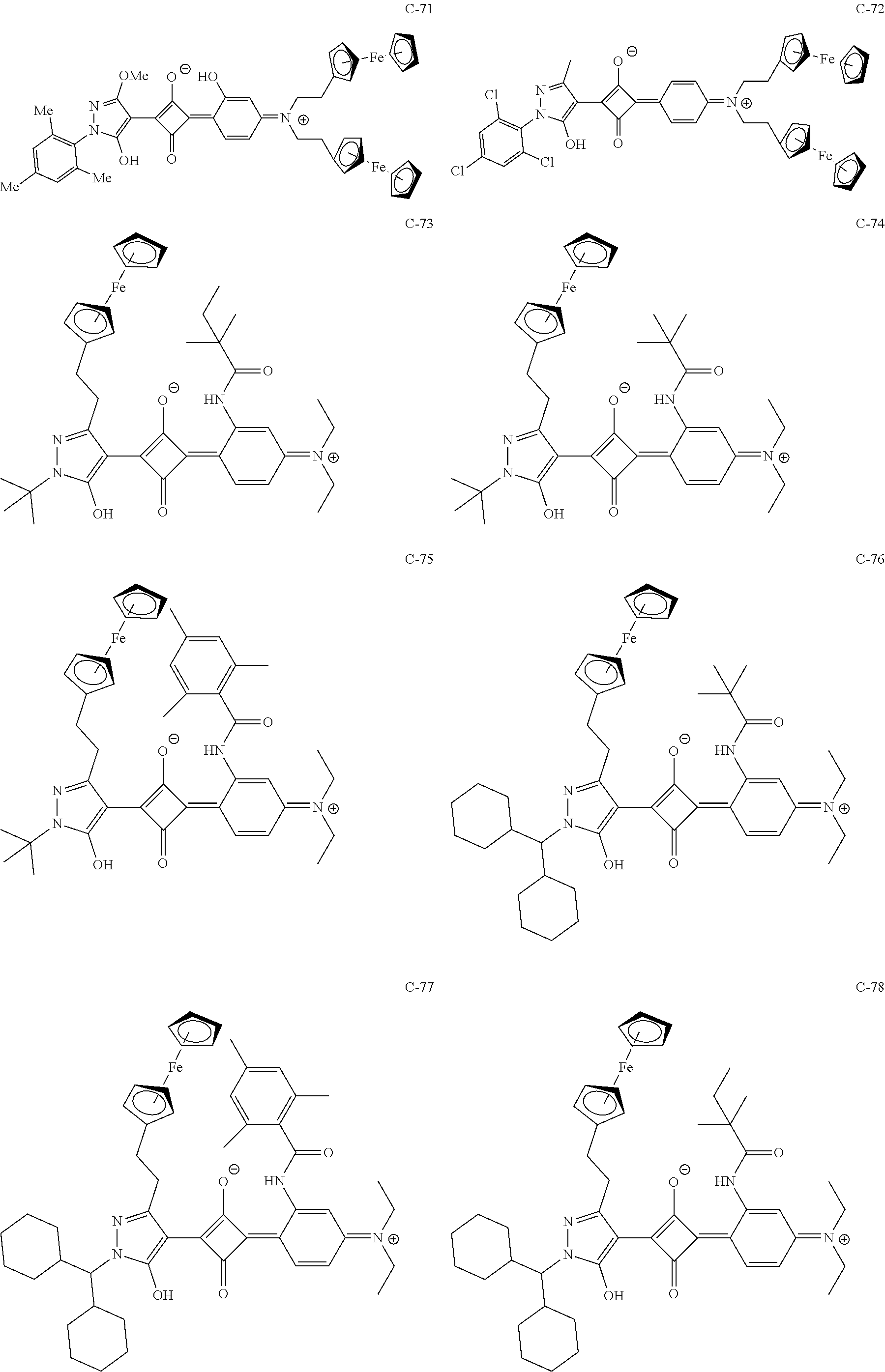

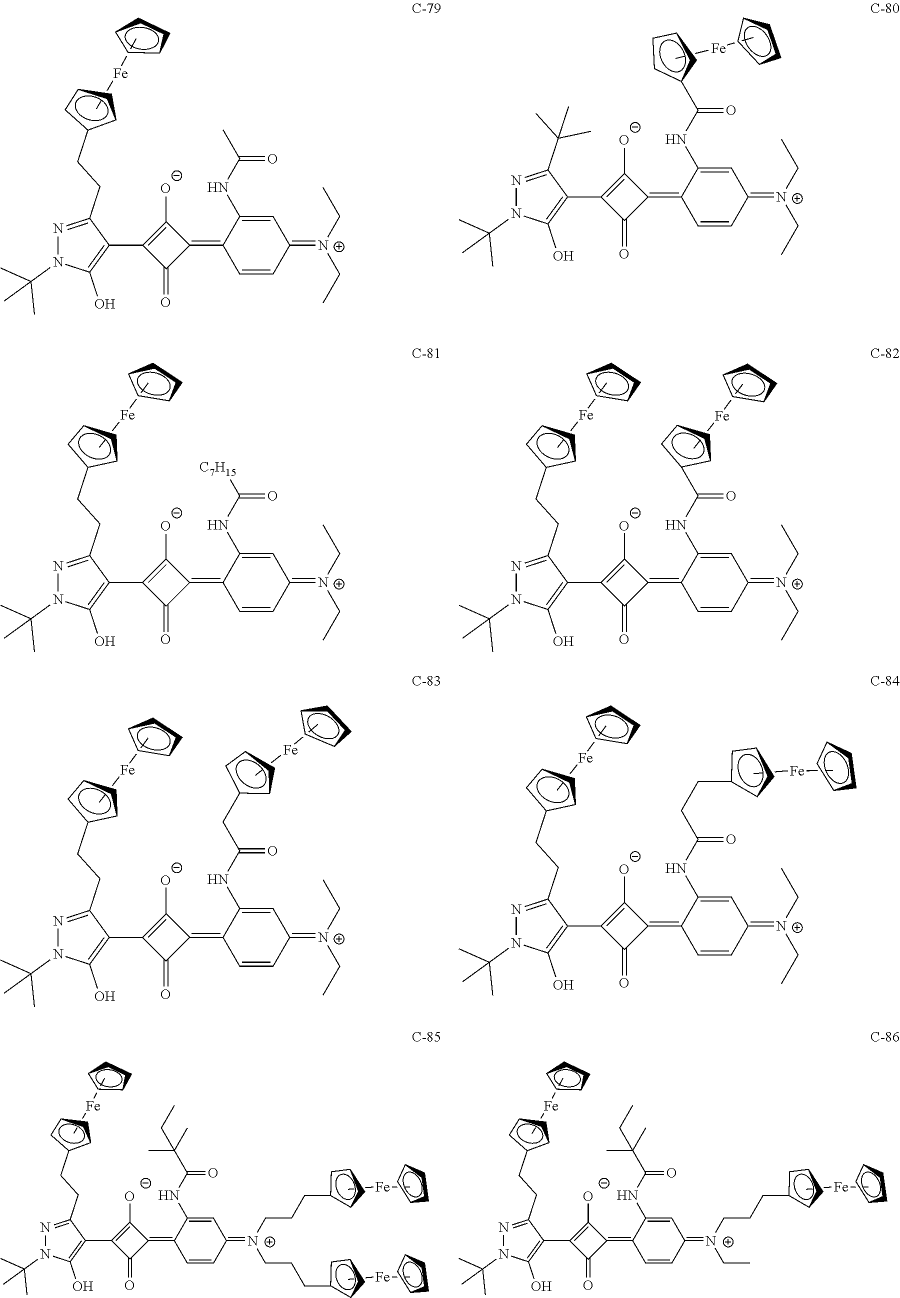

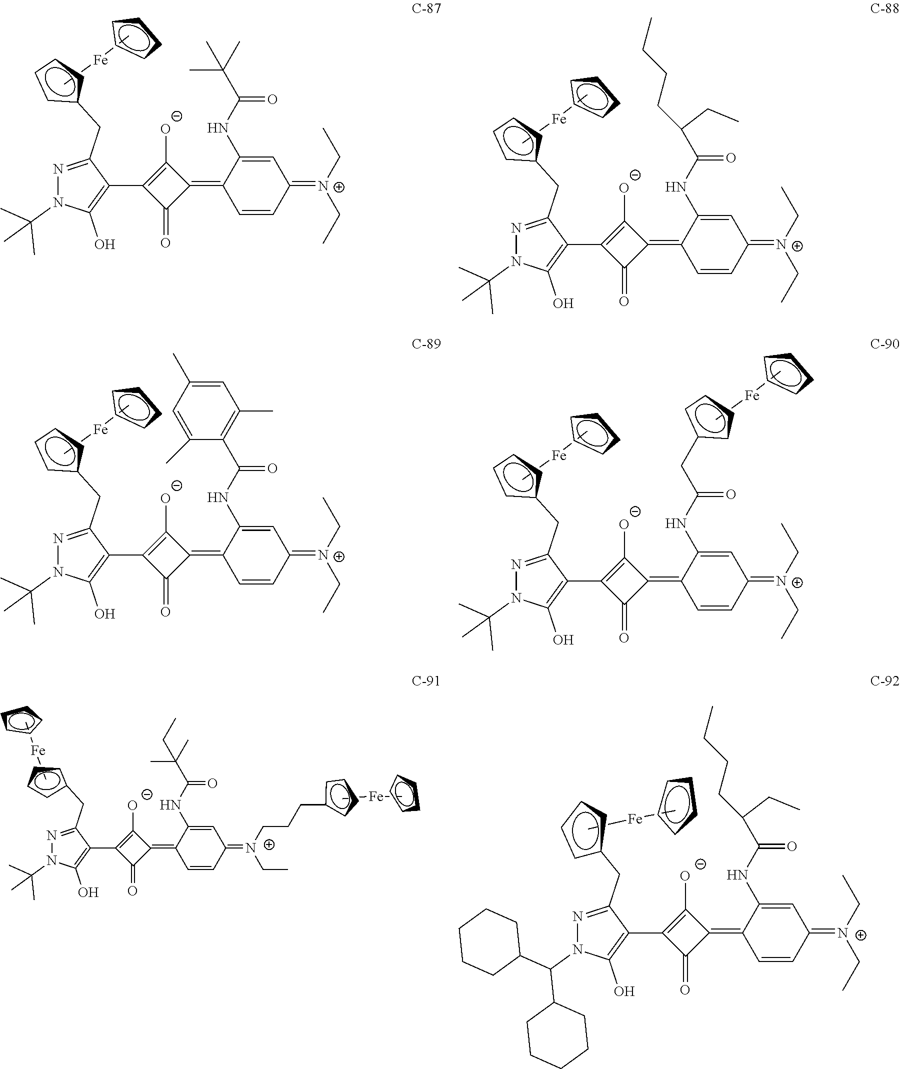

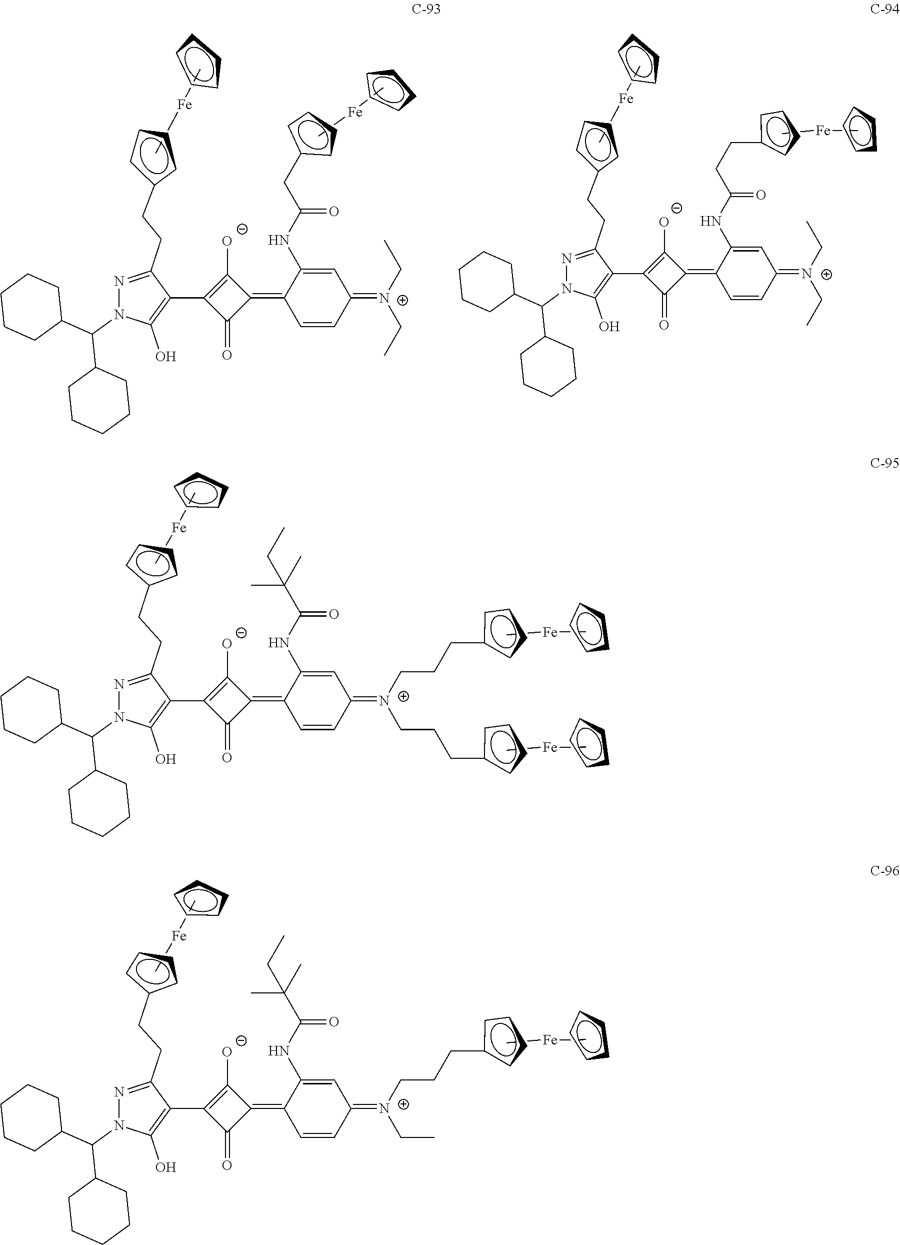

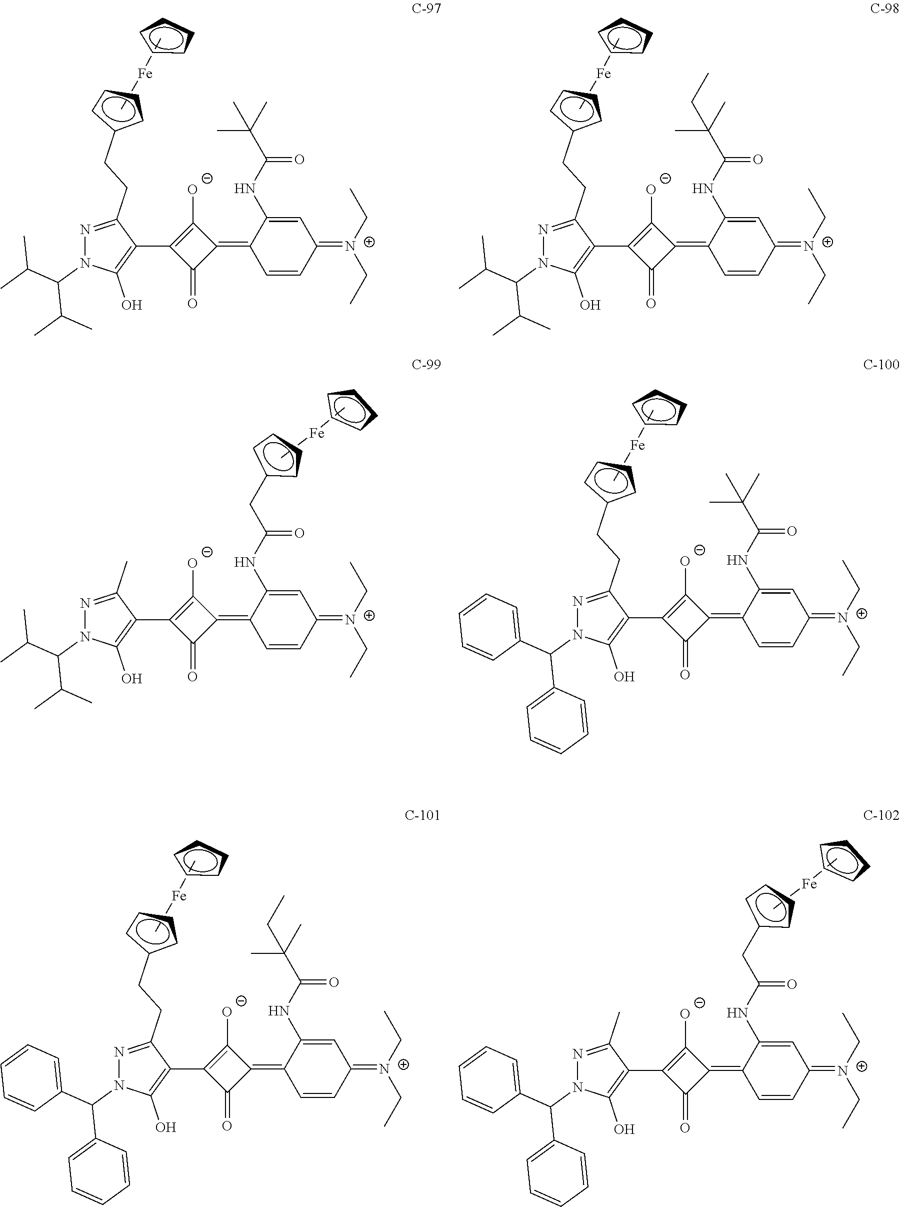

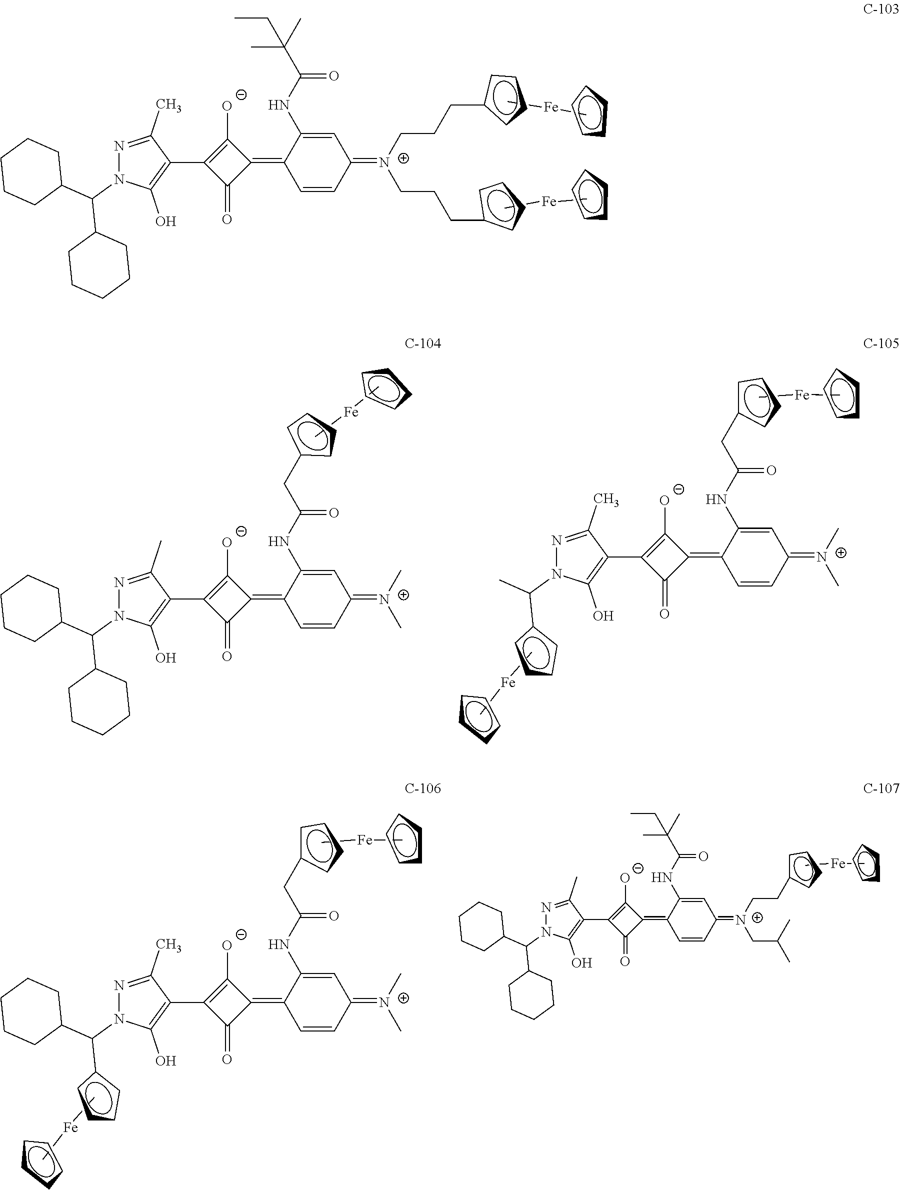

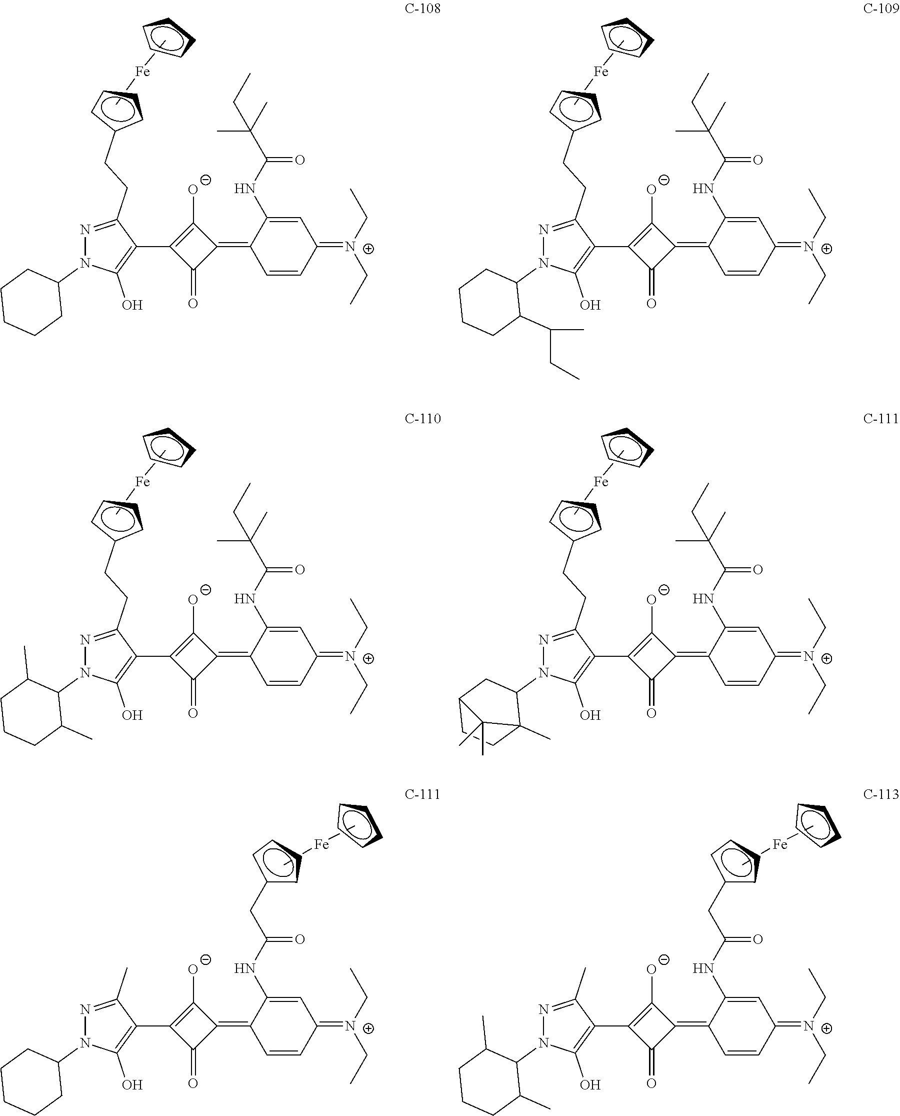

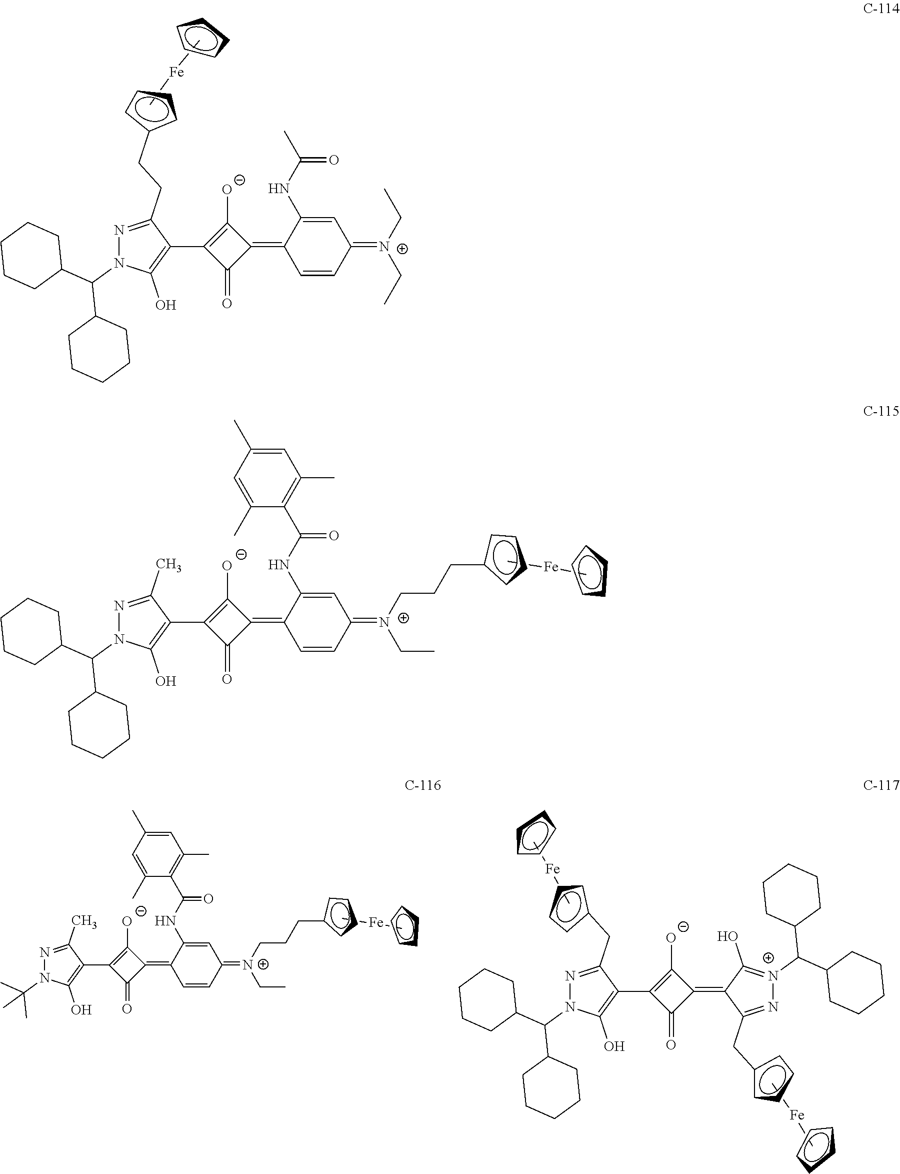

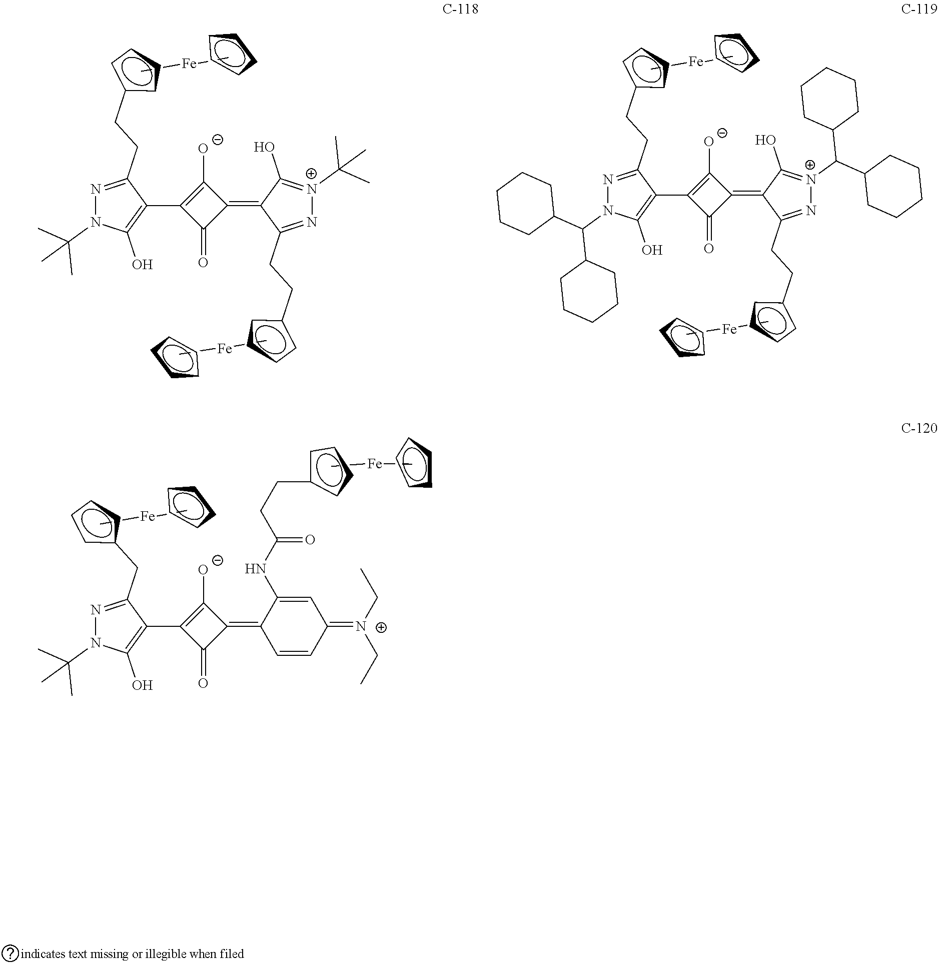

(2) Benzylidene-Based or Cinnamylidene-Based Coloring Agent Represented by General Formula (V)

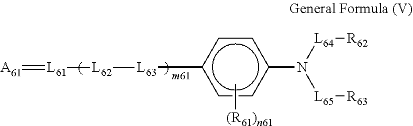

In the formula, A₆₁ represents an acidic nucleus, L₆₁, L₆₂, and L₆₃ each independently represent a methine group which may be substituted, L₆₄ and L₆₅ each independently represent an alkylene group having 1 to 4 carbon atoms. R₆₂ and R₆₃ each independently represent a cyano group, —COOR₆₄ (that is, —C(═O)R₆₄), —CONR₆₅R₆₆ (that is, —C(═O)NR₆₅R₆₆), —COR₆₄ (that is, —C(═O)OR₆₄), —SO₂R₆₄, or —SO₂NR₆₅R₆₆, where R⁶⁴ represents an alkyl group, an alkenyl group, a cycloalkyl group, or an aryl group, R₆₅ and R₆₆ each independently represent a hydrogen atom, an alkyl group, an alkenyl group, a cycloalkyl group, or an aryl group. R₆₁ represents a substituent, m₆₁ is an integer of 0 or 1, and n₆₁ is an integer of 0 to 4.

The compound (coloring agent) represented by General Formula (V) is the same as the benzylidene-based or cinnamylidene-based coloring agent represented by General Formula (V), which is described in [0116] to [0133] of WO2021/132674A. Therefore, regarding the descriptions of each substituent in General Formula (V) and the specific example of the compound represented by General Formula (V), the description of [0118] to [0133] of WO2021/132674A can be applied as it is.

In the light absorption filter according to the embodiment of the present invention, the total content of the dye is preferably 0.10 parts by mass or more, more preferably 0.15 parts by mass or more, still more preferably 0.20 parts by mass or more, particularly preferably 0.25 parts by mass or more, and especially preferably 0.30 parts by mass or more, with respect to 100 parts by mass of the light absorption filter according to the embodiment of the present invention. In a case where the total content of the dye in the light absorption filter according to the embodiment of the present invention is equal to or larger than the above-described preferred lower limit value, a favorable antireflection effect can be obtained.

In addition, in the light absorption filter according to the embodiment of the present invention, the total content of the dyes is generally 50 parts by mass or less, preferably 40 parts by mass or less, more preferably 30 parts by mass or less, still more preferably 15 parts by mass or less, and particularly preferably 10 parts by mass or less, with respect to 100 parts by mass of the light absorption filter according to the embodiment of the present invention.

In the light absorption filter according to the embodiment of the present invention, the content of the squaraine coloring agent represented by General Formula (1) or the benzylidene-based or cinnamylidene-based coloring agent represented by General Formula (V) is preferably 0.01 to 30 parts by mass and more preferably 0.1 to 10 parts by mass with respect to 100 parts by mass of the light absorption filter according to the embodiment of the present invention. It is noted that in the light absorption filter according to the embodiment of the present invention, all of the above coloring agents may be the squaraine coloring agent represented by General Formula (1) or the benzylidene-based or cinnamylidene-based coloring agent represented by General Formula (V).

It is noted that in a case where the dye includes the quencher-embedded coloring agent, the content of the quencher-embedded coloring agent is, from the viewpoint of imparting light absorptance such as the antireflection effect, preferably 0.10 parts by mass or more, more preferably 0.15 parts by mass or more, still more preferably 0.20 parts by mass or more, particularly preferably 0.25 parts by mass or more, and especially preferably 0.30 parts by mass or more, with respect to 100 parts by mass of the resin constituting the light absorption filter according to the embodiment of the present invention. The upper limit value thereof is preferably 45 parts by mass or less, preferably 40 parts by mass or less, more preferably 30 parts by mass or less, still more preferably 15 parts by mass or less, and particularly preferably 10 parts by mass or less.

<Compound that Generates Radical Upon Ultraviolet Irradiation>

The compound that is used in the present invention which generates a radical upon ultraviolet irradiation (hereinafter, also referred to as a “radical generator”) is a compound that generates a radical upon ultraviolet irradiation and is not particularly limited as long as it has a function of decolorizing the dye. In the present invention, it is possible to preferably use a compound that absorbs light and generates a radical (hereinafter, also referred to as a “photoradical generator”). It is noted that the radical generated may be a biradical in addition to the typical radical.

As the photoradical generator, a compound commonly used as a photoradical polymerization initiator a photoradical generator can be used without particular limitation, and examples thereof include an acetophenone generator, a benzoin generator, a benzophenone generator, a phosphine oxide generator, an oxime generator, a ketal generator, an anthraquinone generator, a thioxanthone generator, an azo compound generator, a peroxide generator, a disulfide generator, a lophine dimer generator, an onium salt generator, a borate salt generator, an active ester generator, an active halogen generator, an inorganic complex generator, and a coumarin generator. It is noted that an “XX generator” as the specific example of the photoradical generator may be individually referred to as an “XX compound” or “XX compounds”, and hereinafter, it is referred to as an “XX compound”.

Specific examples, preferred forms, commercially available products, and the like of the photoradical generator are respectively described as the specific examples, preferred forms, commercially available products, and the like of the photoradical initiator in paragraphs [0133] to [0151] of JP2009-098658A, and these can be similarly used suitably in the present invention.

The photoradical generator is preferably a compound that generates a radical upon intramolecular cleavage or a compound that abstracts a hydrogen atom from a compound present in the vicinity thereof to generate a radical, and it is more preferably a compound that abstracts a hydrogen atom from a compound present in the vicinity thereof to generate a radical, from the viewpoint of further improving the decolorization rate.

The above-described compound that generates a radical upon intramolecular cleavage (hereinafter, also referred to as an “intramolecular cleavage type photoradical generator”) means a compound that generates a radical, where the compound absorbing light undergoes bonding cleavage in a homolytic manner.

Examples of the intramolecular cleavage type photoradical generator include an acetophenone compound, a benzoin compound, a phosphine oxide compound, an oxime compound, a ketal compound, an azo compound, a peroxide compound, a disulfide compound, an onium salt compound, a borate salt compound, an active ester compounds, an active halogen compound, an inorganic complex compound, and a coumarin compound. Among these, an acetophenone compound, a benzoin compound, or a phosphine oxide compound, which is a carbonyl compound, is preferable. The Norrish type I reaction is known as a photodecomposition reaction of an intramolecular cleavage type carbonyl compound, and this reaction can be referenced as a radical generation mechanism.

The above-described compound that abstracts a hydrogen atom from a compound present in the vicinity thereof to generate a radical (hereinafter, also referred to as a “hydrogen abstraction type photoradical generator”) means a carbonyl compound in an excited triplet state obtained upon light absorption that abstracts a hydrogen atom from a compound present in the vicinity thereof to generate a radical.

A carbonyl compound is known as the hydrogen abstraction type photoradical generator, and examples thereof include a benzophenone compound, an anthraquinone compound, and a thioxanthone compound. The Norrish type II reaction is known as a photodecomposition reaction of a hydrogen abstraction type carbonyl compound, and this reaction can be referenced as a radical generation mechanism.

Examples of the compound present in the vicinity include various components present in the light absorption filter, such as a resin, a dye, and a radical generator.

The compound present in the vicinity becomes a compound having a radical by a hydrogen atom being abstracted therefrom. Since a dye from which a hydrogen atom has been abstracted by the hydrogen abstraction type photoradical generator becomes an active compound having a radical, the dye may be faded or decolorized through a reaction such as the decomposition of the dye having the radical.

Further, in a case where the hydrogen abstraction type photoradical generator abstracts a hydrogen atom in the molecule, a biradical is generated.

The hydrogen abstraction type photoradical generator is preferably a benzophenone compound from the viewpoint of the quantum yield of the hydrogen abstraction reaction.

(Benzophenone Compound)

Examples of the benzophenone compound used as the hydrogen abstraction type photoradical generator include an alkylbenzophenone compound such as benzophenone, 2-methylbenzophenone, 3-methylbenzophenone, or 4-methylbenzophenone; a benzophenone compound having a halogen atom, such as 2-chlorobenzophenone, 4-chlorobenzophenone, or a 4-bromobenzophenone; a benzophenone compound substituted with a carboxy group or an alkoxycarbonyl group, such as 2-carboxybenzophenone, 2-ethoxycarbonylbenzophenone, benzophenone tetracarboxylic acid, or a tetramethyl ester thereof, a bis(dialkylamino)benzophenone compound (preferably a 4,4′-bis(dialkylamino)benzophenone compound), such as 4,4′-bis(dimethylamino)benzophenone, 4,4′-bis(dicyclohexylamino)benzophenone, 4,4′-bis(diethylamino)benzophenone, or 4,4′-bis(dihydroxyethylamino)benzophenone; and a benzophenone compound substituted with an alkoxy group, such as 4-methoxy-4′-dimethylaminobenzophenone, 4-methoxybenzophenone, or 4,4′-dimethoxybenzophenone.

Among the above-described benzophenone compounds, a benzophenone compound (also referred to as an alkoxybenzophenone compound) substituted with an alkoxy group is preferable from the viewpoint of realizing the achievement of both the light resistance of the unexposed portion and the decolorizing property of the exposed portion at a high level while reducing the molar formulation ratio of the radical generator to the dye.

The number of alkoxy groups contained in the benzophenone compound is preferably 1 to 3 and more preferably 1 or 2.

The moiety of the alkyl chain in the alkoxy group contained in the alkoxybenzophenone compound may be linear or branched. The alkoxy group preferably has 1 to 18 carbon atoms, more preferably 1 to 15 carbon atoms, still more preferably 1 to 12 carbon atoms, and particularly preferably 1 to 6 carbon atoms, among which 1 to 3 carbon atoms are preferable.

Regarding the substitution position of the alkoxy group in the alkoxybenzophenone compound, the alkoxy group is preferably provided at least at the 4-position, more preferably provided at least at the 4-position and the 4′-position, and still more preferably provided at the two positions of the 4-position and the 4′-position, from the viewpoint of realizing the achievement of both the light resistance of the unexposed portion and the decolorizing property of the exposed portion at a higher level while reducing the molar formulation ratio of the radical generator to the dye.

Various examples of the photoradical generator are also described in “Latest UV Curing Technology”, TECHNICAL INFORMATION INSTITUTE CO. LTD., 1991, p. 159, and “Ultraviolet Curing System”, written by Kiyomi Kato, 1989, published by SOGO GIJUTSU CENTER, p. 65 to 148, which can be also suitably used in the present invention.

In the photoradical generator, the maximal absorption wavelength of the ultraviolet ray to be absorbed is preferably in a range of 250 to 400 nm, more preferably in a range of 240 to 400 nm, and still more preferably in a range of 270 to 400 nm.

In a case where the photoradical generator is a benzophenone compound, the wavelength of the absorption maximum attributed to the n-π* transition, which is located on the longest wavelength side, is preferably in a range of 260 to 400 nm and more preferably in a range of 285 to 345 nm. The wavelength of the absorption maximum attributed to π-π*, which is located on the second longest wavelength side, is preferably in a range of 240 to 380 nm and more preferably in a range of 270 to 330 nm. In a case where the absorption maximum wavelength is set in the above range, the light of a light source used at the time of exposure, such as a metal halide lamp, is absorbed well. On the other hand, in a case of being incorporated into a display device, the light absorption filter becomes difficult to absorb an ultraviolet ray incident from the outside, and thus it becomes possible to achieve both the light resistance of the unexposed portion and the decolorizing property of the exposed portion.

Among the benzophenone compounds, examples of the photoradical generator having absorption in a longer wavelength range include an alkoxybenzophenone compound.

In general, the maximal absorption wavelength of the ultraviolet ray absorbed by the photoradical generator is preferably separated by 30 nm or more from the main absorption wavelength band of the dye that has a main absorption wavelength band in a wavelength range of 400 to 700 nm. The upper limit value thereof is not particularly limited.

Examples of the commercially available photocleavage type photoradical generator include “Irgacure 651”, “Irgacure 184”, “Irgacure 819”, “Irgacure 907”, “Irgacure 1870” (a mixed initiator of CGI-403/Irgacure 184=7/3), “Irgacure 500”, “Irgacure 369”, “Irgacure 1173”, “Irgacure 2959”, “Irgacure 4265”, “Irgacure 4263”, “Irgacure 127”, or “OXE01”, all of which are product names, manufactured by BASF SE (formerly Ciba Specialty Chemicals Inc.); additionally, “Kayacure DETX-S”, “Kayacure BP-100”, “Kayacure BDMK”, “Kayacure CTX”, “Kayacure BMS”, “Kayacure 2-EAQ”, “Kayacure ABQ”, “Kayacure CPTX”, “Kayacure EPD”, “Kayacure ITX”, “Kayacure QTX”, “Kayacure BTC”, and “Kayacure MCA”, manufactured by Nippon Kayaku Co., Ltd.; and more additionally “Esacure (KIP100F, KB1, EB3, BP, X33, KT046, KT37, KIP150, and TZT)” manufactured by Sartomer Company Inc. Further, preferred examples thereof include a combination of two or more of these.

In the light absorption filter according to the embodiment of the present invention, the content of the radical generator (preferably a photoradical generator) is preferably 0.01 to 30 parts by mass and more preferably 0.1 to 20 parts by mass with respect to 100 parts by mass of the light absorption filter according to the embodiment of the present invention.

From the viewpoint of further improving the decolorization rate, the formulation amount of the radical generator (preferably a photoradical generator) in the light absorption filter according to the embodiment of the present invention is preferably 0.1 to 20 mol with respect to 1 mol of the dye that has a main absorption wavelength band in a wavelength range of 400 to 700 nm. The lower limit value thereof is more preferably 0.25 mol or more and still more preferably 0.50 mol or more. The upper limit value thereof is more preferably 17.5 mol or less and still more preferably 15 mol or less.

The light absorption filter according to the embodiment of the present invention may contain one kind of the radical generator (preferably a photoradical generator) or may contain two or more kinds thereof.

In the present invention, it is preferable that the radical generator (preferably a photoradical generator) is linked to the dye by a chemical bond. In a case where the radical generator is linked to the dye, the radical generated from the radical generator can efficiently react with the dye, and thus the decolorization speed can be improved.

<Resin>

The resin (hereinafter, also referred to as a “matrix polymer”) contained in the light absorption filter according to the embodiment of the present invention preferably contains a polymer having both a partial structure having a high affinity (hereinafter, also referred to as a “high affinity part”) and a partial structure having a low affinity (hereinafter, also referred to as a “low affinity part”), with respect to the dye and the compound (the radical generator) that generates a radical upon ultraviolet irradiation.

Examples of the one form of the light absorption filter according to the embodiment of the present invention include a light absorption filter containing a resin, a dye having a main absorption wavelength band in a wavelength range of 400 to 700 nm, and a compound that generates a radical upon ultraviolet irradiation, where the resin is composed of a polymer having both a high affinity part and a low affinity part with respect to the dye and the radical generator.

The high affinity part makes it possible to disperse (preferably dissolve) the dye and the radical generator (preferably a photoradical generator) in the light absorption filter.

On the other hand, the low affinity part surrounds the high affinity part to confine the dye and the radical generator (preferably a photoradical generator) in the high affinity part, and increases the efficiency of the reaction between the radical generated from the radical generator (preferably a photoradical generator) and the dye, or the efficiency of generating a dye having a radical due to the fact that the photoradical generator abstracts a hydrogen atom (a hydrogen radical) from the dye, thereby exhibiting an effect of promoting decolorization, and it may be any part having a relatively low affinity with respect to the high affinity part.

In the polymer having both a high affinity part and a low affinity part, the high affinity part and the low affinity part are not particularly limited as long as they have a relative relationship which exhibits the above-described function.

Regarding the high affinity part, it is possible to discuss the high affinity with respect to the dye and the radical generator, for example, using fd, fp, and fh, which are represented by the following expressions.

fd={δd/(δd+δp+δh)}×100

fp={δd/(δd+δp+δh)}×100

fh={δd/(δd+δp+δh)}×100

Here, δd, δp, and δh respectively represent, in the following order, the term of London dispersion force, the term of dipole-dipole force, and the term of hydrogen bonding force of the solubility parameters calculated according to the Hoy method, respectively. A specific calculation method of fd will be described later.

In the fd, fp, and fh of the high affinity part, and the fd, fp, and fh of the dye and the radical generator, which are calculated based on the above expressions, any one of the absolute value of the difference in fd, the absolute value of the difference in fp, and the absolute value of the difference in fd is preferably 15 or less, and any one thereof is more preferably 13 or less. The lower limit value of each of the absolute value of the difference in fd, the absolute value of the difference in fp, and the absolute value of the difference in fh is not particularly limited and it may be, for example, a value of 1 or more.

—Term δd Corresponding to London Dispersion Force—

The term δd of the London dispersion force refers to δd obtained for the Amorphous Polymers described in the column “2) Method of Hoy (1985, 1989)” on pages 214 to 220 of the document “Properties of Polymers 3^{rd}, ELSEVIER, (1990)”, and is calculated according to the description in the column of the document. It is noted that regarding a dye containing a ferrocenyl group, the calculation shall be carried out for a structure in which the ferrocenyl group is replaced with a hydrogen atom.

—Term δp of Dipole-Dipole Force—

The term δp of the dipole-dipole force refers to Sp obtained for Amorphous Polymers described in the column “2) Method of Hoy (1985, 1989)” on pages 214 to 220 of the document “Properties of Polymers 3^{rd}, ELSEVIER, (1990)”, and is calculated according to the description in the column of the document.

It is noted that regarding a dye containing a ferrocenyl group, the calculation shall be carried out for a structure in which the ferrocenyl group is replaced with a hydrogen atom.

—Term δh of Hydrogen Bonding Force—

The term δh of the hydrogen bonding force refers to δh obtained for the Amorphous Polymers described in the column “2) Method of Hoy (1985, 1989)” on pages 214 to 220 of the document “Properties of Polymers 3^{rd}, ELSEVIER, (1990)”, and is calculated according to the description in the column of the document.

It is noted that regarding a dye containing a ferrocenyl group, the calculation shall be carried out for a structure in which the ferrocenyl group is replaced with a hydrogen atom.

In the present invention, the polymer having both a high affinity part and a low affinity part is preferably a block copolymer having a block consisting of a high affinity part and a block consisting of a low affinity part. In the block copolymer, the dye and the radical generator (preferably a photoradical generator) can be confined in a region (phase) having a size of a width of several tens of nanometers by forming a micro-phase separation structure, and thus the reaction efficiency between the radical and the dye or the generation efficiency of the dye having a radical can be further increased.

In the present invention, the high affinity part is not particularly limited as long as it has a desired light transmittance (preferably, the light transmittance is 80% or more in a visible range in a wavelength range of 400 to 800 nm). However, it preferably includes a polystyrene structure from the viewpoint of exhibiting affinity with respect to a wide variety of dyes and radical generators (preferably photoradical generators).

In addition, the low affinity part is not particularly limited either as long as it has a desired light transmittance (preferably, the light transmittance is 80% or more in a visible range in a wavelength range of 400 to 800 nm). However, it is preferably a structure in which a linear or branched alkylene group is linked, and it is particularly preferable to include a structure in which a polydiene structure is hydrogenated, from the viewpoint of low compatibility with dye and the radical generator (preferably a photoradical generator). It is noted that the structure in which a polydiene structure is hydrogenated is meant to include, in addition to a structure in which all double bonds in the polydiene structure are hydrogenated, a structure in which double bonds of the main chain portion in the polydiene structure is selectively hydrogenated.

In the present invention, the polymer having both a high affinity part and a low affinity part preferably forms an ordered structure by micro-phase separation. Among the above, an ordered structure such as a spherical phase or a cylinder phase is particularly preferable in that the dye and the radical generator (preferably a photoradical generator) can be confined in a smaller space.

In the present invention, the ratio of the high affinity part to the low affinity part is preferably in a range of 10/90 to 80/20 and more preferably 20/80 to 70/30 in terms of high affinity part/low affinity part, based on the weight ratio. In a case of adjusting the ratio of the high affinity part to the low affinity part to be within the above-described range, it is possible to achieve both a high decolorization speed and heat resistance. From the viewpoint of improving the decolorization rate, the ratio base on the weight ratio is still more preferably in a range of high affinity part/low affinity part=20/80 to 60/40, and particularly preferably in a range of high affinity part/low affinity part=20/80 to 50/50.

A preferred example of the polymer having both a high affinity part and a low affinity part is, for example, preferably a hydrogenated styrene-diene-based copolymer (hereinafter, also referred to as a “hydrogenated styrene-diene-based copolymer”) such as a hydrogenated styrene-butadiene-styrene block copolymer (SEBS) obtained by hydrogenating a styrene-butadiene-styrene block copolymer (SBS) or hydrogenated styrene-isoprene-styrene block copolymer (SEPS) obtained by hydrogenating a styrene-isoprene-styrene block copolymer (SIS). In addition, a copolymer in which double bonds of the main chain portion of the butadiene constituting the SBS are selectively hydrogenated is also preferably mentioned. It is noted that as the polymer having both a high affinity part and a low affinity part, only one kind of polymer may be used, or two or more kinds thereof may be used.

As the hydrogenated styrene-diene-based copolymer, a commercially available product may be used. Examples thereof include “TUFTEC H series” and “TUFTEC P series” manufactured by Asahi Kasei Corporation (formerly Asahi Kasei Chemicals Corporation) and “KRATON G series” manufactured by Shell Japan Ltd. (all are SEBS), “DYNARON” manufactured by JSR Corporation (hydrogenated styrene-butadiene random copolymer), and “SEPTON” manufactured by Kuraray Co., Ltd. (SEPS), all of which are product names. In addition, examples of the modified hydrogenated styrene-diene-based copolymer include “TUFTEC M series” manufactured by Asahi Kasei Corporation (formerly Asahi Kasei Chemicals Corporation), “EPOFRIEND” manufactured by Daicel Corporation, “Polar Group Modified DYNARON” manufactured by JSR Corporation, and “RESEDA” manufactured by ToaGosei Co., Ltd., all of which are product names.

The light absorption filter according to the embodiment of the present invention preferably contains a polyphenylene ether resin in addition to the hydrogenated styrene-diene-based copolymer. In a case of containing the polystyrene resin and the polyphenylene ether resin together, the physical crosslinking in the hydrogenated styrene-diene-based copolymer can be strengthened, and the heat resistance can be improved.

As the polyphenylene ether resin, XYRON S201A, XYRON S202A, XYRON S203A (all of which are product names), and the like, manufactured by Asahi Kasei Corporation, can be preferably used. In addition, a resin which the polystyrene resin and the polyphenylene ether resin are mixed in advance may also be used. As the mixed resin of the polystyrene resin and the polyphenylene ether resin, for example, XYRON 1002H, XYRON 1000H, XYRON 600H, XYRON 500H, XYRON 400H, XYRON 300H, XYRON 200H (all of which are product names), and the like manufactured by Asahi Kasei Corporation can be preferably used.

In a case where the hydrogenated styrene-diene-based copolymer and the polyphenylene ether resin are used in combination in the light absorption filter according to the embodiment of the present invention, the mass ratio of both resins is preferably 99/1 to 50/50, more preferably 98/2 to 60/40, and still more preferably 95/5 to 70/30, in terms of hydrogenated styrene-diene-based copolymer/polyphenylene ether resin. In a case where the formulation ratio of the polyphenylene ether resin is set in the above-described preferred range, the light absorption filter according to the embodiment of the present invention can have sufficient toughness, and the solvent can be properly volatilized in a case where a film is formed with a solution.

<Resin Composed of Polymer Having Partial Structure that Generates Radical Upon Ultraviolet Irradiation>

A resin composed of a polymer including a partial structure that generates a radical upon ultraviolet irradiation can also be preferably used as a resin that is contained in the light absorption filter according to the embodiment of the present invention.

Examples of the embodiment of the light absorption filter according to the embodiment of the present invention include a light absorption filter containing a dye having a main absorption wavelength band in a wavelength range of 400 to 700 nm and a resin composed of a polymer including a partial structure that generates a radical upon ultraviolet irradiation.

Hereinafter, the resin according to the embodiment of the present invention, which is composed of a polymer including a partial structure that generates a radical upon ultraviolet irradiation, will be described in detail.

The polymer including a partial structure that generates a radical upon ultraviolet irradiation is not particularly limited as long as it is a polymer that includes a partial structure that generates a radical upon ultraviolet irradiation and has a function of decolorizing the dye. In the present invention, a polymer having a partial structure that absorbs light and generates a radical can be preferably used. It is noted that the radical generated may be a biradical in addition to the typical radical.

The partial structure that generates a radical upon ultraviolet irradiation is preferably a partial structure that generates a radical upon intramolecular cleavage or a partial structure that abstracts a hydrogen atom from a compound present in the vicinity thereof to generate a radical, and it is more preferably a partial structure that abstracts a hydrogen atom from a compound present in the vicinity thereof to generate a radical, from the viewpoint of further improving the quenching rate.

The partial structure that generates a radical upon intramolecular cleavage and the partial structure that abstracts a hydrogen atom from a compound present in the vicinity thereof to generate a radical are preferably partial structures respectively derived from the above-described compound that generates a radical upon intramolecular cleavage and the above-described compound that abstracts a hydrogen atom from a compound present in the vicinity thereof to generate a radical.

Among the above, a polymer having a partial structure derived from a benzophenone compound (also referred to as an alkoxybenzophenone compound) substituted with an alkoxy group is preferable from the viewpoint of being capable of realizing the achievement of both the light resistance of the unexposed portion and the decolorizing property of the exposed portion while reducing the molar formulation ratio of the radical generator to the dye.

Various polymers can be used as the polymer including a partial structure that generates a radical upon ultraviolet irradiation, and examples thereof include an alkyl (meth)acrylic polymer and a polymer having an aromatic ring in the side chain. However, it is preferably a polymer having an aromatic structure (an aromatic ring) in the side chain, more preferably a (meth)acrylic polymer having an aromatic ring in the side chain, and still more preferably a (meth)acrylic polymer containing a constitutional unit having an aromatic ring in the side chain, from the viewpoint that the decrease in the molecular weight of the polymer due to ultraviolet irradiation is difficult to occur. A polymer having an aromatic structure (an aromatic ring) in the side chain is classified into a polymer including a partial structure that generates a radical upon intramolecular cleavage.

Hereinafter, in a case where the (meth)acrylic polymer is referred to, it refers to a polymer containing at least one of a constitutional unit derived from (meth)acrylic acid or a constitutional unit derived from (meth)acrylic acid ester. In the present invention, the “main chain” indicates the relatively longest bond chain in the polymer that constitutes the resin, and the “side chain” indicates an atomic group branched from the main chain.

Examples of the monomer that forms a constitutional unit having an aromatic ring in the side chain include benzyl acrylate, benzyl methacrylate, naphthyl acrylate, naphthyl methacrylate, naphthyl methyl acrylate, and naphthyl methyl methacrylate.

The content of the constitutional unit having an aromatic ring in the side chain is preferably 5% by mass to 100% by mass, more preferably 10% by mass to 100% by mass, still more preferably 20% by mass to 100% by mass, and particularly preferably 50% by mass to 100% by mass, with respect to the total mass of the resin.

From the viewpoint of controlling the glass transition temperature and the like, the polymer including a partial structure that generates a radical upon ultraviolet irradiation may contain a constitutional unit having an alkyl group having 1 to 14 carbon atoms. The constitutional unit having an alkyl group having 1 to 14 carbon atoms is preferably an alkyl (meth)acrylate having an alkyl group having 1 to 14 carbon atoms, and examples thereof include methyl (meth)acrylate, ethyl (meth)acrylate, n-propyl (meth)acrylate, isopropyl (meth)acrylate, n-butyl (meth)acrylate, t-butyl (meth)acrylate, sec-butyl (meth)acrylate, pentyl (meth)acrylate, 2-ethylhexyl (meth)acrylate, 2-ethylbutyl (meth)acrylate, n-octyl (meth)acrylate, isooctyl (meth)acrylate, isononyl (meth)acrylate, lauryl (meth)acrylate, isobornyl (meth)acrylate, and tetradecyl (meth)acrylate.

The constitutional unit having an alkyl group having 1 to 14 carbon atoms may be used alone, or two or more kinds thereof may be used in combination. It is preferable that an amount of 0% by mass to 95% by mass of the constitutional unit having an alkyl group having 1 to 14 carbon atoms is contained with respect to the total mass of the resin.

From the viewpoint of polarity adjustment, the polymer including a partial structure that generates a radical upon ultraviolet irradiation may contain a constitutional unit having an alicyclic skeleton.

The monomer that forms a constitutional unit having an alicyclic skeleton is preferably an alkyl (meth)acrylate having an alicyclic skeleton, and examples thereof include dicyclopentanyl (meth)acrylate, cyclohexyl (meth)acrylate, and isobornyl (meth)acrylate.

In a case where the polymer including a partial structure that generates a radical upon ultraviolet irradiation contains a constitutional unit having an alicyclic skeleton, the content of the constitutional unit having an alicyclic skeleton is preferably 1% by mass to 90% by mass and more preferably 5% by mass to 80% by mass with respect to the total mass of the resin.

In addition, from the viewpoint of polarity adjustment, the polymer including a partial structure that generates a radical upon ultraviolet irradiation may contain a constitutional unit derived from (meth)acrylic acid. The content of the constitutional unit derived from (meth)acrylic acid is preferably 0% by mass to 70% by mass and more preferably 0% by mass to 60% by mass with respect to the total mass of the polymer.

The weight-average molecular weight (Mw) of the polymer including a partial structure that generates a radical upon ultraviolet irradiation is preferably 10,000 or more, more preferably 10,000 to 200,000, and still more preferably 15,000 to 150,000.

(Crosslinking)

It is preferable that the polymer contained in the light absorption filter according to the embodiment of the present invention, which has both a high affinity part and a low affinity part with respect to the dye and radical generator described above, has a crosslinking structure. In a case of having a crosslinking structure, it is possible to suppress the diffusion of the dye and the compound that generates a radical upon ultraviolet irradiation, to the outside of the high affinity part, it is possible to further improve the decolorization rate, and an effect of sharpening the boundary between the ultraviolet irradiated part, and the ultraviolet non-irradiated part at the time is exhibited when the mask exposure is carried out.

As the crosslinking structure, any one of a physical crosslink or a chemical crosslink can be preferably used.

In a case where the polymer having both a high affinity part and a low affinity part is a hydrogenated styrene-polydiene block copolymer, the styrene domain functions as a physical crosslinking moiety, which is preferable. In addition, in a case of adding a polyphenylene ether resin to the hydrogenated styrene-polydiene block copolymer, the glass transition temperature of the styrene domain can be increased, the physical crosslinking of the styrene domain can be strengthened, and the heat resistance can be improved.

Further, although chemical crosslinking can be carried out in any one of the high affinity part or the low affinity part, a method of introducing a crosslinking structure into the low affinity part is preferable from the viewpoint of confining the dye or the compound that generates a radical upon ultraviolet irradiation in the high affinity part. For example, in a case where the styrene-butadiene copolymer is used as a polymer having both a high affinity part and a low affinity part, the polydiene structure can be crosslinked by radical polymerization. In addition, a maleic acid anhydride-modified hydrogenated styrene-polydiene block copolymer such as TUFTEC M1913 or TUFTEC M1943 manufactured by Asahi Kasei Corporation or KRATON FG1901 manufactured by Shell Japan Limited (all of which are product names) is used as the polymer having both a high affinity part and a low affinity part, the maleic acid anhydride moiety can also be used as the crosslinking site. In addition, a difunctional or higher functional monomer having low polarity and high compatibility with the low affinity part can be added to increase the degree of crosslinking.

(Peelability Control Resin Component)

The light absorption filter according to the embodiment of the present invention can contain, as a resin component, a component that controls the peelability (a peelability control resin component) in a case of being produced by a method including a step of peeling the light absorption filter according to the embodiment of the present invention from a release film, among the methods of manufacturing the light absorption filter according to the embodiment of the present invention described later, which is preferable. By controlling the peelability of the light absorption filter according to the embodiment of the present invention from the release film, it is possible to prevent a peeling mark from being left on the light absorption filter according to the embodiment of the present invention after peeling, and it is possible to cope with various processing speeds in the peeling step. As a result, a preferable effect can be obtained for improving the quality and productivity of the light absorption filter according to the embodiment of the present invention.

The peelability control resin component is not particularly limited and can be appropriately selected depending on the kind of the release film. In a case where a polyester-based polymer film is used as the release film as described later, for example, a polyester resin (also referred to as a polyester-based additive) is suitable as the peelability control resin component. In a case where a cellulose acylate-based film is used as the release film, for example, a hydrogenated polystyrene-based resin (also referred to as a hydrogenated polystyrene-based additive) is suitable as the peelability control resin component.

For the polyester-based additive and the hydrogenated polystyrene-based additive, the descriptions regarding the polyester-based additive and hydrogenated polystyrene-based additive described in [0177] to [0188] of WO2021/132674A can be applied as they are.

The content of the peelability control resin component in the light absorption filter according to the embodiment of the present invention is preferably 0.05% by mass or more, and more preferably 0.1% by mass or more in the matrix polymer. In addition, the upper limit value thereof is preferably 25% by mass or less, more preferably 20% by mass or less, and still more preferably 15% by mass or less. From the viewpoint of obtaining proper adhesiveness, the above-described preferred range is preferable.

<Other Components>

In addition to the above-described dye, the above-described compound that generates a radical upon ultraviolet irradiation, and the above-described matrix polymer, the absorption filter according to the embodiment of the present invention may contain an antifading agent, a matting agent, a leveling agent (a surfactant), and the like.

<Antifading Agent>

It is preferable that the antifading agent according to the present invention does not inhibit the decolorization due to ultraviolet irradiation but has an effect of suppressing the dye decomposition due to visible light.

The compound represented by General Formula (IV) below can be preferably used as the antifading agent.

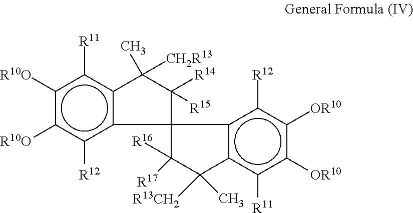

In Formula (IV), R¹⁰ represents an alkyl group, an alkenyl group, an aryl group, a heterocyclic group, or a group represented by R¹⁸CO—, R¹⁹SO₂—, or R²⁰NHCO—. Here, R¹⁸, R¹⁹, and R²⁰ each independently represent an alkyl group, an alkenyl group, an aryl group, or a heterocyclic group. R¹¹ and R¹² each independently represent a hydrogen atom, a halogen atom, an alkyl group, an alkenyl group, an alkoxy group, or an alkenyloxy group, and R¹³, R¹⁴, R¹⁵, R¹⁶, and R¹⁷ each independently represent a hydrogen atom, an alkyl group, an alkenyl group, or an aryl group.

However, the alkyl group in R¹⁰ to R²⁰ includes an aralkyl group.

The compound represented by General Formula (IV) is the same as the compound represented by General Formula (IV) described in [0215] to [0221] of WO2021/221122A. Therefore, for the descriptions of each substituent in General Formula (IV) and the specific example of the compound represented by General Formula (IV), the description of [0217] to [0221] of WO2021/221122A can be applied as it is.

As the antifading agent, the compound represented by General Formula [III] can also be preferably used.

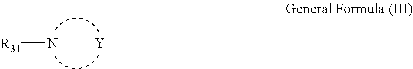

In General Formula [III], R₃₁ represents an aliphatic group or an aromatic group, and Y represents a non-metal atomic group necessary for forming a 5- to 7-membered ring with a nitrogen atom.

The compound represented by General Formula [III] is the same as the compound represented by General Formula [III] described in [0223] to [0227] of WO2021/221122A. Therefore, for the descriptions of each substituent in General Formula [III] and the specific example of the compound represented by General Formula [III], the description of [0225] to [0227] of WO2021/221122A can be applied as it is.

In addition, in addition to the above-described specific examples, specific examples of the compound represented by General Formula [III] above include exemplary compounds B-1 to B-65 described on pages 8 to 11 of JP1990-167543A (JP-H2-167543A), and exemplary compounds (1) to (120) described on pages 4 to 7 of JP1988-95439A (JP-S63-95439A).

The content of the antifading agent in the light absorption filter according to the embodiment of the present invention is preferably 1% to 15% by mass, more preferably 5% to 15% by mass, still more preferably 5% to 12.5% by mass, and particularly preferably 10% to 12.5% by mass in 100% by mass of the total mass of the light absorption filter according to the embodiment of the present invention.

In a case where the antifading agent is contained within the above-described preferred range, the light absorption filter according to the embodiment of the present invention can improve the light resistance of the dye (the coloring agent) without causing side effects such as discoloration of the wavelength selective absorption layer.

(Matting Agent)

In order to impart sliding properties and prevent blocking, fine particles may be added to the surface of the light absorption filter according to the embodiment of the present invention as long as the effect of the present invention is not impaired. As the fine particles, silica (silicon dioxide, SiO₂) of which the surface is coated with a hydrophobic group and which has an aspect of secondary particles is preferably used. As the fine particles, in addition to or instead of silica, fine particles of titanium dioxide, aluminum oxide, zirconium oxide, calcium carbonate, talc, clay, calcined kaolin, calcined calcium silicate, hydrated calcium silicate, aluminum silicate, magnesium silicate, and calcium phosphate may be used. Examples of the commercially available product of the fine particles include the R972 or NX90S (product name, both manufactured by Nippon Aerosil Co., Ltd.).

The fine particles function as a so-called matting agent, and the addition of the fine particles forms minute unevenness on the surface of the light absorption filter according to the embodiment of the present invention. Due to the unevenness, even in a case where the light absorption filters according to the embodiment of the present invention overlap with each other or the light absorption filter according to the embodiment of the present invention and other films overlap with each other, the films do not stick to each other and sliding properties are secured.

In a case where the light absorption filter according to the embodiment of the present invention contains a matting agent as fine particles, the effect of improving sliding properties and blocking properties is particularly large in the fine unevenness due to the protrusions in which fine particles protrude from the filter surface in a case where there are 10⁴/mm² or more of protrusions having a height of 30 nm or more.

It is preferable to apply the matting agent fine particles particularly onto the surface layer in order to improve the blocking properties and the sliding properties. Examples of the method of applying fine particles onto the surface layer include methods such as multilayer casting and coating.

The content of the matting agent in the light absorption filter according to the embodiment of the present invention is appropriately adjusted depending on the intended purpose.

However, in a case where a gas barrier layer described later is provided in the light absorption filter according to the embodiment of the present invention, the above-described matting agent fine particles are preferably applied onto the surface of the light absorption filter in contact with the gas barrier layer as long as the effect of the present invention is not impaired.

(Leveling Agent)

A leveling agent (surfactant) can be appropriately mixed with the light absorption filter according to the embodiment of the present invention. As the leveling agent, a commonly used compound can be used, and a fluorine-containing surfactant is particularly preferable. Specific examples thereof include the compounds described in paragraphs [0028] to [0056] of JP2001-330725A. Further, as the commercially available product, MEGAFACE F (product name) series manufactured by DIC Corporation can also be used.

The content of the leveling agent in the light absorption filter according to the embodiment of the present invention is appropriately adjusted depending on the intended purpose.

The light absorption filter according to the embodiment of the present invention may contain, in addition to the above components, a low-molecular plasticizer, an oligomer-based plasticizer, a retardation modifier, a deterioration preventing agent, a peeling accelerating agent, an infrared absorbing agent, an antioxidant, a filler, a compatibilizer.

Further, the light absorption filter according to the embodiment of the present invention may contain the reaction accelerating agent or the reaction retarder described in paragraphs [0020] and [0021] of JP1997-286979A (JP-H09-286979A).

<Manufacturing Method for Light Absorption Filter>

The light absorption filter according to the embodiment of the present invention can be produced by a solution film-forming method, a melt extrusion method, or a method of forming a coating layer on a base material film (release film) (coating method) according to any method, according to a conventional method, and stretching can also be appropriately combined. The light absorption filter according to the embodiment of the present invention is preferably produced by a coating method.

For the solution film-forming method and melt extrusion method described above, the descriptions regarding the solution film-forming method and the melt extrusion method in [0197] to [0203] of WO2021/132674A can be applied as they are.

(Coating Method)

In the coating method, a solution of a material of the light absorption filter is applied to a release film to form a coating layer. A release agent or the like may be appropriately applied to the surface of the release film in advance in order to control the adhesiveness to the coating layer. The coating layer can be used by peeling off the release film after being laminated with another member while interposing an adhesive layer in a later step. Any adhesive can be appropriately used as the adhesive constituting the adhesive layer. The whole release film can be appropriately stretched in a state where a solution of the material of the light absorption filter is applied on the release film or in a state where a coating layer is laminated on the release film.

The solvent that is used for the solution of the material of the light absorption filter can be appropriately selected from the viewpoints that the material of the light absorption filter can be dissolved or dispersed, that a uniform surface shape can be easily achieved during the coating step and drying step, liquid storability can be secured, and that a proper saturated vapor pressure is provided.

—Addition of Dye (Coloring Agent) and/or Radical Generator (Preferably Photoradical Generator)—

The timing of adding the dye and/or the radical generator (preferably the photoradical generator) to the material of the light absorption filter is not particularly limited as long as they are added at the time of film formation. For example, the dye may be added at the time of synthesizing the matrix polymer or may be mixed with the material of the light absorption filter at the time of preparing the coating liquid for the material of the light absorption filter.

—Release Film—

The release film that is used for forming the light absorption filter according to the embodiment of the present invention by a coating method or the like preferably has a film thickness of 5 to 100 μm, more preferably 10 to 75 μm, and still more preferably 15 to 55 μm. In a case where the film thickness is equal to or larger than the above-described preferred lower limit value, sufficient mechanical strength can be easily secured, and failures such as curling, wrinkling, and buckling are less likely to occur. In addition, in a case where the film thickness is equal to or smaller than the above-described preferred upper limit value, in the storage of a multi-layer film of the release film and the light absorption filter according to the embodiment of the present invention, for example, in the form of a long roll, the surface pressure applied to the multi-layer film is easily adjusted to be in an appropriate range, and adhesion defect is less likely to occur.

The surface energy of the release film is not particularly limited, and by adjusting the relationship between the surface energy of the material of the light absorption filter according to the embodiment of the present invention or the coating solution and the surface energy of the surface of the release film on which the light absorption filter according to the embodiment of the present invention is to be formed, the adhesive force between the light absorption filter according to the embodiment of the present invention and the release film can be adjusted. In a case where the surface energy difference is reduced, the adhesive force tends to increase, and in a case where the surface energy difference is increased, the adhesive force tends to decrease, and thus the surface energy can be set appropriately.

The surface energy of the release film can be calculated from the contact angle value between water and methylene iodide using the Owen's method. For the measurement of the contact angle, for example, DM901 (contact angle meter, manufactured by Kyowa Interface Science Co., Ltd.) can be used.

The surface energy of the surface of the release film on which the light absorption filter according to the embodiment of the present invention is to be formed is preferably 41.0 to 48.0 mN/m and more preferably 42.0 to 48.0 mN/m. In a case where the surface energy is equal to or larger than the above-described preferred lower limit value, the evenness of the thickness of the light absorption filter according to the embodiment of the present invention is increased. In a case where the surface energy is equal to or smaller than the above-described preferred upper limit value, it is easy to control the peeling force of the light absorption filter according to the embodiment of the present invention from the release film within an appropriate range.

The surface unevenness of the release film is not particularly limited, and depending on the relationship between the surface energy of the light absorption filter according to the embodiment of the present invention surface, the hardness, and the surface unevenness, and the surface energy and hardness of the surface of the release film opposite to the side on which the light absorption filter according to the embodiment of the present invention is formed, for example, in order to prevent adhesion defect in a case where the multi-layer film of the release film and the light absorption filter according to the embodiment of the present invention is stored in the form of a long roll, the surface unevenness of the release film can be adjusted. In a case where the surface unevenness is increased, adhesion defect tends to be suppressed, and in a case where the surface unevenness is reduced, the surface unevenness of the light absorption filter according to the embodiment of the present invention tends to be decreased and the haze of the light absorption filter according to the embodiment of the present invention tends to be small. Thus, the surface unevenness can be set appropriately.

For such a release film, any material and film can be appropriately used. Specific examples of the material include a polyester-based polymer (including polyethylene terephthalate-based film), an olefin-based polymer, a cycloolefin-based polymer, a (meth)acrylic polymer, a cellulose-based polymer, and a polyamide-based polymer. In addition, a surface treatment can be appropriately carried out for the intended purpose of adjusting the surface properties of the release film. For example, a corona treatment, a room temperature plasma treatment, or a saponification treatment can be carried out to decrease the surface energy, and a silicone treatment, a fluorine treatment, an olefin treatment, or the like can be carried out to raise the surface energy.

—Peeling Force Between Light Absorption Filter According to Embodiment of Present Invention and Release Film—

In a case where the light absorption filter according to the embodiment of the present invention is formed by a coating method, the peeling force between the light absorption filter according to the embodiment of the present invention and the release film can be controlled by adjusting the material of the light absorption filter according to the embodiment of the present invention, the material of the release film, and the internal distortion of the light absorption filter according to the embodiment of the present invention. The peeling force can be measured by, for example, a test of peeling off the release film in a direction of 90°, and the peeling force in a case of being measured at a rate of 300 mm/min is preferably 0.001 to 5 N/25 mm, more preferably 0.01 to 3 N/25 mm, and still more preferably 0.05 to 1 N/25 mm. In a case where the peeling force is equal to or larger than the above-described preferred lower limit value, peeling off the release film in a step other than the peeling step can be prevented, and in a case where the peeling force is equal to or smaller than the above-described preferred upper limit value, peeling failure in the peeling step (for example, zipping and cracking of the light absorption filter according to the embodiment of the present invention) can be prevented.

<Film Thickness of Light Absorption Filter According to Embodiment of Present Invention>

The film thickness of the light absorption filter according to the embodiment of the present invention is not particularly limited, and it is preferably 0.5 to 18 μm, more preferably 0.8 to 12 μm, and still more preferably 1 to 8 μm. In a case where the film thickness is equal to or smaller than the above-described preferred upper limit value, the decrease in the degree of polarization due to the fluorescence emitted by a dye (a coloring agent) can be suppressed by adding the dye to the thin film at a high concentration. In addition, the effect of the quencher is likely to be exhibited. On the other hand, in a case where the film thickness is equal to or larger than the above-described preferred lower limit value, it becomes easy to maintain the evenness of the in-plane absorbance.

In the present invention, the film thickness of 0.5 to 18 μm means that the thickness of the light absorption filter according to the embodiment of the present invention is within a range of 0.5 to 18 μm in a case of being measured at any portion. The same applies to the film thicknesses of 0.8 to 12 μm and 1 to 8 μm. The film thickness can be measured with an electronic micrometer manufactured by Anritsu Corporation.

<Absorbance of Light Absorption Filter of According to Embodiment According to Embodiment of Present Invention>

In the light absorption filter according to the embodiment of the present invention, the absorbance at the maximal absorption wavelength at which the highest absorbance is exhibited at a wavelength of 400 to 700 nm (hereinafter, also simply referred to as “Ab (λₘₐₓ)”) is preferably 0.3 or more, more preferably 0.5 or more, still more preferably 0.7 or more, and particularly preferably 0.8 or more.

However, the absorbance of the light absorption filter according to the embodiment of the present invention can be adjusted by the kind, adding amount, or film thickness of the dye.

The light absorption filter according to the embodiment of the present invention has a decolorization rate upon ultraviolet irradiation of preferably 35% or more, more preferably 45% or more, still more preferably 50% or more, and particularly preferably 55% or more, among which 70% or more is preferable. The upper limit value thereof is not particularly limited, and it is preferably 100%.

The decolorization rate is calculated according to the following expression using the values of Ab (λₘₐₓ) before and after the ultraviolet irradiation test.

Decolorization rate (%)=100−(Ab(λₘₐₓ) after ultraviolet irradiation/Ab(λₘₐₓ)before ultraviolet irradiation)×100

Here, in the ultraviolet irradiation test, the light absorption filter is irradiated with an ultraviolet ray of a predetermined irradiation dose at room temperature (25° C.) using an air-cooled metal halide lamp (manufactured by EYE GRAPHICS Co., Ltd.) at 160 W/cm under atmospheric pressure (101.33 kPa).

The absorbance, the ultraviolet irradiation test, and the decolorization rate can be measured and calculated according to the methods described in Examples.

In addition, the light absorption filter according to the embodiment of the present invention hardly causes absorption (secondary absorption) derived from a new coloration structure associated with the decomposition of the coloring agent.

For example, the presence or absence of the absorption derived from the new coloration structure associated with the decomposition of the coloring agent can be checked based on the ratio of the absorbance at a specific wavelength to the above Ab (λₘₐₓ). As the specific wavelength, a wavelength at which the coloring agent before ultraviolet irradiation seldom exhibits absorption but new absorption due to the decomposition of the coloring agent is observed is selected.

As a specific example, as described in Examples described later, the presence or absence of the absorption derived from a new coloration structure associated with the decomposition of the coloring agent can be checked based on the ratio of the absorbance at a wavelength of 450 nm to the above Ab (λₘₐₓ) (hereinafter, also simply referred to as “Ab (450)”). That is, it is meant that the smaller the value obtained by subtracting the ratio of the following (I) from the ratio of the following (II), the less frequently the absorption derived from the new coloration structure associated with the decomposition of the coloring agent occurs. This value is preferably less than 8.5%, more preferably 7.0% or less, still more preferably 5.0% or less, particularly preferably 3.0% or less, among which 1.0% or less is preferable. The lower limit value thereof is not particularly limited; however, it is practically −10% or more and preferably −6% or more from the viewpoint of making valid the evaluation related to the presence or absence of the secondary absorption associated with the decomposition of the coloring agent.
(I) Ab (450) before ultraviolet irradiation/Ab (λₘₐₓ)×100% before ultraviolet irradiation
(II) Ab (450) after ultraviolet irradiation/Ab (λₘₐₓ) before ultraviolet irradiation×100%

The checking of the presence or absence of the absorption derived from the new coloration structure associated with the decomposition of the coloring agent can be carried out by the measurement and the calculation according to the method described in Examples.

The light absorption filter according to the embodiment of the present invention can exhibit an excellent quenching property in a case where both the above-described decolorization rate and the above-described value for checking the presence or absence of the absorption derived from the new coloration structure associated with the decomposition of the coloring agent satisfy a preferred range.

The light absorptive portion having a light absorption effect in the optical filter according to the embodiment of the present invention preferably satisfies the above description of Ab (λₘₐₓ) related to the light absorption filter according to the embodiment of the present invention.

<Treatment of Light Absorption Filter According to Embodiment of Present Invention>

The light absorption filter according to the embodiment of the present invention may be subjected to a hydrophilic treatment by any of glow discharge treatment, corona discharge treatment, or alkali saponification treatment, and a corona discharge treatment is preferably used. It is also preferable to apply the method disclosed in JP1994-94915A (JP-H6-94915A) and JP1994-118232A (JP-H6-118232A).

As necessary, the obtained film may be subjected to a heat treatment step, a superheated steam contact step, an organic solvent contact step, or the like. In addition, a surface treatment may be appropriately carried out.

Further, as the pressure sensitive adhesive layer, a layer consisting of a pressure sensitive adhesive composition in which a (meth)acrylic resin, a styrene-based resin, a silicone-based resin, or the like is used as a base polymer, and a crosslinking agent such as an isocyanate compound, an epoxy compound, or an aziridine compound is added thereto can be applied.

Preferably, the description regarding the pressure sensitive adhesive layer in the OLED display device described later can be applied.

<Gas Barrier Layer>

The light absorption filter according to the embodiment of the present invention may have a gas barrier layer on at least one surface. In a case where the light absorption filter according to the embodiment of the present invention has a gas barrier layer, the light absorption filter according to the embodiment of the present invention can be made to be a light absorption filter that achieves both excellent photoquenching property and excellent light resistance and can be suitably used in the production of an optical filter described later.

The material that forms the gas barrier layer is not particularly limited, and examples thereof include an organic material (preferably a crystalline resin) such as polyvinyl alcohol or polyvinylidene chloride, an organic-inorganic hybrid material such as a sol-gel material, and an inorganic material such as SiO₂, SiOₓ, or SiON, SiNₓ, or Al₂O₃. The gas barrier layer may be a single layer or a multi-layer. In the case of a multi-layer, examples thereof include configurations such as an inorganic dielectric multi-layer film and a multi-layer film obtained by alternately laminating organic materials and inorganic materials.

In a case where the light absorption filter according to the embodiment of the present invention includes the gas barrier layer at least on a surface that comes into contact with air in a case where the light absorption filter according to the embodiment of the present invention is used, it is possible to suppress a decrease in the absorption intensity of the dye in the light absorption filter according to the embodiment of the present invention. As long as the gas barrier layer is provided at an interface of the light absorption filter according to the embodiment of the present invention in contact with air, the gas barrier layer may be provided on only one surface of the light absorption filter according to the embodiment of the present invention, or may be provided on both surfaces.

Among the above, in a case of a configuration in which the gas barrier layer contains a crystalline resin, the gas barrier layer contains a crystalline resin, and it is preferable that the thickness of the layer is 0.1 μm to 10 μm and the oxygen permeability of the layer is 60 cc/m²·day·atm or less.

In the gas barrier layer, the “crystalline resin” is a resin having a melting point that undergoes a phase transition from a crystal to a liquid in a case where the temperature is raised, and it can impart gas barrier properties related to oxygen gas to the gas barrier layer.

(Crystalline Resin)

The crystalline resin contained in the gas barrier layer is a crystalline resin having gas barrier properties, and it can be used without particular limitation as long as a desired oxygen permeability can be imparted to the gas barrier layer.

Examples of the crystalline resin include polyvinyl alcohol and polyvinylidene chloride, and the polyvinyl alcohol is preferable from the viewpoint that a crystalline portion can effectively suppress the permeation of gas.

The polyvinyl alcohol may be modified or may not be modified. Examples of the modified polyvinyl alcohol include modified polyvinyl alcohol into which a group such as an acetoacetyl group and a carboxy group is introduced.

The saponification degree of the polyvinyl alcohol is preferably 80.0% by mol or more, more preferably 90.0% by mol or more, still more preferably 97.0% by mol or more, and particularly preferably 98.0% by mol or more, from the viewpoint of further enhancing the oxygen gas barrier properties. The upper limit value thereof is not particularly limited, and it is practically 99.99% by mol or less. The saponification degree of the polyvinyl alcohol is a value calculated based on the method described in JIS K 6726 1994.

The gas barrier layer may contain any component generally contained in the gas barrier layer as long as the effect of the present invention is not impaired. For example, in addition to the above crystalline resin, organic-inorganic hybrid materials such as an amorphous resin material and a sol-gel material, and inorganic materials such as SiO₂, SiOₓ, SiON, SiNₓ, and Al₂O₃ may be contained.

Further, the gas barrier layer may contain a solvent such as water and an organic solvent derived from a manufacturing step as long as the effect of the present invention is not impaired.

The content of the crystalline resin in the gas barrier layer is, for example, preferably 90% by mass or more and more preferably 95% by mass or more in 100% by mass of the total mass of the gas barrier layer. The upper limit value thereof is not particularly limited, and it can be set to 100% by mass.

The oxygen permeability of the gas barrier layer is preferably 60 cc/m²·day·atm or less, more preferably 50 cc/m²·day·atm or less, still more preferably 30 cc/m²·day·atm or less, and particularly preferably 10 cc/m²·day·atm or less. Among the above, it is preferably 5 cc/m²·day·atm or less and most preferably 1 cc/m²·day·atm or less. The practical lower limit value thereof is 0.001 cc/m²·day·atm or more, and it is preferably, for example, more than 0.05 cc/m²·day·atm. In a case where the oxygen permeability is within the above-described preferred range, the light resistance can be further improved.

The oxygen permeability of the gas barrier layer is a value measured based on the gas permeability test method based on JIS K 7126-2 2006. As the measuring device, for example, an oxygen permeability measuring device OX-TRAN2/21 (product name) manufactured by MOCON can be used. The measurement conditions are set to a temperature of 25° C. and a relative humidity of 50%.

For the oxygen permeability, (fm)/(s·Pa) can be used as the SI unit. It is possible to carry out the conversion by (1 fm)/(s·Pa)=8.752 (cc)/(m²·day·atm). fm is read as femtometer and represents 1 fm=10⁻¹⁵ m.

The thickness of the gas barrier layer is preferably 0.5 μm to 5 μm, and more preferably 1.0 μm to 4.0 μm, from the viewpoint of further improving the light resistance.

The thickness of the gas barrier layer is measured by a method of capturing a cross-sectional image using a field emission scanning electron microscope S-4800 (product name) manufactured by Hitachi High-Technologies Corporation.

The degree of crystallinity of the crystalline resin contained in the gas barrier layer is preferably 25% or more, more preferably 40% or more, and still more preferably 45% or more. The upper limit value thereof is not particularly limited, and it is practically 55% or less and preferably 50% or less.

The degree of crystallinity of the crystalline resin contained in the gas barrier layer is a value measured and calculated according to the following method based on the method described in J. Appl. Pol. Sci., 81, 762 (2001).

Using a differential scanning calorimeter (DSC), a temperature of a sample peeled from the gas barrier layer is raised at 10° C./min over the range of 20° C. to 260° C., and a heat of fusion 1 is measured. Further, as a heat of fusion 2 of the perfect crystal, the value described in J. Appl. Pol. Sci., 81, 762 (2001) is used. Using the obtained heat of fusion 1 and heat of fusion 2, the degree of crystallinity is calculated according to the following expression.

[Degree of crystallinity (%)]=([heat of fusion 1]/[heat of fusion 2])×100

The heat of fusion 1 and heat of fusion 2 may have the same unit, which is generally Jg⁻¹.

<Manufacturing Method for Gas Barrier Layer>

The method of forming the gas barrier layer is not particularly limited, and examples thereof include a forming method according to a conventional method, for example in a case of an organic material, according to a casting method such as spin coating or slit coating. In addition, examples thereof can include a method of bonding a commercially available resin gas barrier film or a resin gas barrier film produced in advance to the light absorption filter according to the embodiment of the present invention. In addition, in a case of an inorganic material, examples thereof include a plasma enhanced chemical vapor deposition (CVD) method, a sputtering method, and a vapor deposition method.

<Optical Functional Film>

The light absorption filter according to the embodiment of the present invention may appropriately have the gas barrier layer or any optical functional film as long as the effect of the present invention is not impaired.

The optional optical functional film is not particularly limited in terms of any of optical properties and materials, and a film containing (or containing as a main component) at least any of a cellulose ester resin, an acrylic resin, a cyclic olefin resin, and a polyethylene terephthalate resin can be preferably used. It is noted that an optically isotropic film or an optically anisotropic phase difference film may be used.

For the above optional optical functional films, for example, Fujitac TD80UL (manufactured by FUJIFILM Corporation) or the like can be used as a film containing a cellulose ester resin.

Regarding the optional optical functional film, as those containing an acrylic resin, an optical film containing a (meth)acrylic resin containing a styrene-based resin described in JP4570042B, an optical film containing a (meth)acrylic resin having a glutarimide ring structure in a main chain described in JP5041532B, an optical film containing a (meth)acrylic resin having a lactone ring structure described in JP2009-122664A, and an optical functional film containing a (meth)acrylic resin having a glutaric anhydride unit described in JP2009-139754A can be used.

Further, regarding the optional optical functional films, as those containing a cyclic olefin resin, cyclic olefin-based resin film described in paragraphs [0029] and subsequent paragraphs of JP2009-237376A, and cyclic olefin resin film containing an additive reducing Rth described in JP4881827B, and JP2008-063536A can be used.

[Optical Filter]

The optical filter according to the embodiment of the present invention is obtained by subjecting the light absorption filter according to the embodiment of the present invention to mask exposure by ultraviolet irradiation.

The optical filter according to the embodiment of the present invention has a light absorptive portion having a light absorption effect and a portion in which light absorption properties have been eliminated (a light absorption property-eliminated portion) in response to a mask exposure pattern (hereinafter, also referred to as a “mask pattern”).

That is, in a case where the light absorption filter according to the embodiment of the present invention is subjected to mask exposure by ultraviolet irradiation, the masked portion of the light absorption filter according to the embodiment of the present invention is not exposed and present as a light absorptive portion having a light absorption effect, whereas the unmasked portion is exposed and becomes a light absorption property-eliminated portion.

The light absorptive portion can exhibit a desired absorbance.

Further, the light absorption property-eliminated portion can exhibit optical characteristics close to colorlessness since the light absorption filter according to the embodiment of the present invention exhibits an excellent decolorization rate and secondary absorption seldom occurs in association with the dye decomposition.

<Manufacturing Method for Optical Filter>

The optical filter according to the embodiment of the present invention can be obtained by irradiating the light absorption filter according to the embodiment of the present invention with an ultraviolet ray to carry out mask exposure.

The mask pattern can be appropriately adjusted so that the optical filter according to the embodiment of the present invention having a desired pattern consisting of a light absorptive portion and a light absorption property-eliminated portion can be obtained.

The conditions of ultraviolet irradiation can be appropriately adjusted so that the optical filter according to the embodiment of the present invention having a light absorption property-eliminated portion can be obtained. For example, the pressure condition can be treated under atmospheric pressure (101.33 kPa), the temperature condition can be treated without heating at room temperature (10° C. to 30° C.) or the like, the lamp output can be set to 80 to 320 W/cm, and an air-cooled metal halide lamp, a high-pressure mercury lamp, an ultra-high-pressure mercury lamp, or the like can be used as a lamp to be used. The irradiation dose can be set to 200 to 5,000 mJ/cm².

In particular, in a case where the distance between the light absorption filter according to the embodiment of the present invention and the ultraviolet irradiation lamp is set to a distance of 50 to 120 cm or the like, and exposure is carried out with a low illuminance of 5 to 50 mW/cm² or the like, an optical filter having excellent reproducibility of a mask pattern can be obtained, which is preferable.

The optical filter according to the embodiment of the present invention may have an optical functional film described in the light absorption filter according to the embodiment of the present invention.

Further, the optical filter according to the embodiment of the present invention may have a layer containing an ultraviolet absorbing agent. As the ultraviolet absorbing agent, a commonly used compound can be used without particular limitation, and examples thereof include an ultraviolet absorbing agent in the ultraviolet absorbing layer described later. The resin constituting the layer containing the ultraviolet absorbing agent is also not particularly limited, and examples thereof include a resin in the ultraviolet absorbing layer described later.

The content of the ultraviolet absorbing agent in the layer containing the ultraviolet absorbing agent is appropriately adjusted depending on the intended purpose.

<<Manufacturing Method for Laminate>>

In a case where the above-described gas barrier layer is provided in the light absorption filter according to the embodiment of the present invention, for example, a method of directly producing the above-described gas barrier layer on the light absorption filter according to the embodiment of the present invention produced according to the above-described production method is included. In this case, it is also preferable to apply a corona treatment to the surface of the light absorption filter according to the embodiment of the present invention to which the gas barrier layer is provided.

Further, in a case where the above-described optional optical functional film is provided, it is also preferable to carry out bonding while interposing a pressure sensitive adhesive layer. For example, it is also preferable that a gas barrier layer is provided on the light absorption filter according to the embodiment of the present invention and then bonded to an optical functional film while interposing a pressure sensitive adhesive layer.

[OLED Display Device]

The organic electroluminescent display device according to the embodiment of the present invention (referred to as an organic electroluminescence (EL) display device or an organic light emitting diode (OLED) display device, and abbreviated as an OLED display device in the present invention) includes the optical filter according to the embodiment of the present invention.

As another configuration of the OLED display device according to the embodiment of the present invention, the configuration of the generally used OLED display device can be used without particular limitation, as long as the optical filter according to the embodiment of the present invention is included. The configuration example of the OLED display device according to the embodiment of the present invention is not particularly limited, and examples thereof include a display device including glass, a layer containing a thin film transistor (TFT), an OLED display element, a barrier film, a color filter, glass, a pressure sensitive adhesive layer, the optical filter according to the embodiment of the present invention, and a surface film, in order from the opposite side to external light.

The OLED display element has a configuration in which an anode electrode, a light emitting layer, and a cathode electrode are laminated in this order. In addition to the light emitting layer, a hole injection layer, a hole transport layer, an electron transport layer, an electron injection layer, and the like are included between the anode electrode and the cathode electrode. In addition, for example, the description in JP2014-132522A can also be referenced.

Further, as the color filter, in addition to a typical color filter, a color filter in which quantum dots are laminated can also be used.

A resin film can be used instead of the above glass.

In the OLED display device according to the embodiment of the present invention, it is preferable that the optical filter according to the embodiment of the present invention is bonded to the glass (the base material) while interposing a pressure sensitive adhesive layer, on a surface positioned opposite to the side of the external light.

For the pressure sensitive adhesive layer, the descriptions related to the pressure sensitive adhesive layer and the forming method in the OLED display device, which are described in [0239] to [0292] of WO2021/132674A, can be applied as they are.

[Liquid Crystal Display Device]

The liquid crystal display device according to the embodiment of the present invention includes the optical filter according to the embodiment of the present invention.

The optical filter according to the embodiment of the present invention may be used as at least one of a polarizing plate-protective film or a pressure sensitive adhesive layer as described later, or it may be included in a backlight unit that is used in the liquid crystal display device.

It is preferable that the liquid crystal display device includes the optical filter according to the embodiment of the present invention, a polarizing plate including a polarizer and a polarizing plate-protective film, a pressure sensitive adhesive layer, and a liquid crystal cell, where it is preferable that the polarizing plate is bonded to the liquid crystal cell with a pressure sensitive adhesive layer being interposed. In the liquid crystal display device, the optical filter according to the embodiment of the present invention may also serve as the polarizing plate-protective film or the pressure sensitive adhesive layer. That is, the liquid crystal display device is divided into a case where the liquid crystal display device includes a polarizing plate including a polarizer and the optical filter (polarizing plate-protective film) according to the embodiment of the present invention, a pressure sensitive adhesive layer, and a liquid crystal cell, and a case where the liquid crystal display device includes a polarizing plate including a polarizer and a polarizing plate-protective film, the optical filter (pressure sensitive adhesive layer) according to the embodiment of the present invention, and a liquid crystal cell.

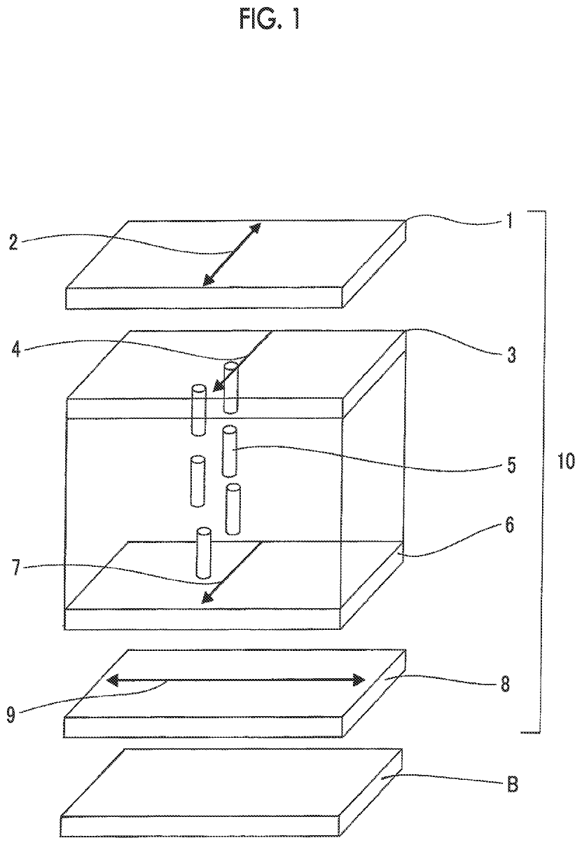

FIG. 1 is a schematic view illustrating an example of the liquid crystal display device according to the embodiment of the present invention. In FIG. 1, a liquid crystal display device 10 consists of a liquid crystal cell having a liquid crystal layer 5 and having a liquid crystal cell upper electrode substrate 3 and a liquid crystal cell lower electrode substrate 6, which are respectively disposed above and below the liquid crystal layer 5, and an upper polarizing plate 1 and a lower polarizing plate 8, which are respectively disposed on both sides of the liquid crystal cell. A color filter layer may be laminated on the upper electrode substrate 3 or the lower electrode substrate 6. On the rear surface of the liquid crystal display device 10, a backlight is disposed. As a light source of the backlight, those described in the above backlight unit can be used.

Each of the upper polarizing plate 1 and the lower polarizing plate 8 has a configuration in which each of them is laminated such that a polarizer is sandwiched between two polarizing plate-protective films, and in the liquid crystal display device 10, at least one of the polarizing plates may include the optical filter according to the embodiment of the present invention.

In addition, in the liquid crystal display device 10, the liquid crystal cell may be bonded to the polarizing plates (the upper polarizing plate 1 and/or the lower polarizing plate 8) with a pressure sensitive adhesive layer (not illustrated in the drawing) being interposed. In this case, the optical filter according to the embodiment of the present invention may also serve as the above-described pressure sensitive adhesive layer.

The liquid crystal display device 10 includes an image direct vision-type liquid crystal display, an image projection-type liquid crystal display device, and a light modulation-type liquid crystal display device. An active matrix liquid crystal display device in which a three-terminal or two-terminal semiconductor element such as TFT or MIM is used is effective for the present invention. In addition, a passive matrix liquid crystal display device represented by an STN mode which is called as the time division driving is also effective.

In a case where the optical filter according to the embodiment of the present invention is included in the backlight unit, the polarizing plate of the liquid crystal display device may be a general polarizing plate (a polarizing plate that does not include the optical filter according to the embodiment of the present invention) or may be a polarizing plate that includes the optical filter according to the embodiment of the present invention. In addition, the pressure sensitive adhesive layer may be a typical pressure sensitive adhesive layer (not the optical filter according to the embodiment of the present invention) or may be a pressure sensitive adhesive layer formed of the optical filter according to the embodiment of the present invention.

The IPS mode liquid crystal display device described in paragraphs 128 to 136 of JP2010-102296A is preferable as the liquid crystal display device according to the embodiment of the present invention except that the optical filter according to the embodiment of the present invention is used.

<Polarizing Plate>

The polarizing plate that is used in the present invention includes a polarizer and at least one polarizing plate-protective film.

The polarizing plate that is used in the present invention is preferably a polarizing plate having a polarizer and polarizing plate-protective films on both surfaces of the polarizer, and it is preferable that at least one surface of the polarizer includes the optical filter according to the embodiment of the present invention as the polarizing plate-protective film. The surface of the polarizer opposite to the surface having the optical filter according to the embodiment of the present invention (the polarizing plate-protective film according to the embodiment of the present invention) may have a general polarizing plate-protective film.

The film thickness of the polarizing plate-protective film is preferably 1 μm or more and 120 μm or less, and more preferably 2 μm or more and 100 μm or less. A thinner film is preferable since in a case of being incorporated in the liquid crystal display device, the display unevenness after elapse of time in high temperature and high humidity is less likely to occur. On the other hand, in a case where the film is too thin, it is difficult to transport the film stably at the time of producing the film and producing the polarizing plate. In a case where the optical filter according to the embodiment of the present invention also serves as the polarizing plate-protective film, it is preferable that the thickness of the optical filter satisfies the above-described range.

For the polarizing plate that is used in the present invention, the descriptions related to the performance, the shape, the configuration, the polarizer, the method of laminating the polarizer and the polarizing plate-protective film, the functionalization of the polarizing plate, and the like regarding the polarizing plate described in [0299] to [0309] of WO2021/132674A can be applied as they are.

<Pressure Sensitive Adhesive Layer>

In the liquid crystal display device according to the embodiment of the present invention, the polarizing plate is preferably bonded to the liquid crystal cell with a pressure sensitive adhesive layer being interposed. The optical filter according to the embodiment of the present invention may also serve as the pressure sensitive adhesive layer. In a case where the optical filter according to the embodiment of the present invention does not serve as the pressure sensitive adhesive layer, a typical pressure sensitive adhesive layer can be used as the pressure sensitive adhesive layer.

The pressure sensitive adhesive layer is not particularly limited as long as the polarizing plate can be bonded to the liquid crystal cell, and for example, an acrylic type, a urethane type, polyisobutylene, or the like is preferable.

In a case where the optical filter according to the embodiment of the present invention also serves as a pressure sensitive adhesive layer, the pressure sensitive adhesive layer includes the coloring agent and the binder resin, and further contains a crosslinking agent, a coupling agent, or the like to impart adhesiveness.

In a case where the optical filter additionally serves as a pressure sensitive adhesive layer, the pressure sensitive adhesive layer includes the binder resin in an amount of preferably 90% to 100% by mass and preferably 95% to 100% by mass. The content of the coloring agent is as described above.

The thickness of the pressure sensitive adhesive layer is not particularly limited; however, it is preferably 1 to 50 μm and more preferably 3 to 30 μm.

<Liquid Crystal Cell>

The liquid crystal cell is not particularly limited, and a typical liquid crystal cell can be used.

[Self-Luminous Display Device]

The self-luminous display device according to the embodiment of the present invention is a self-luminous type display device including a light emitting diode as a light emitting source, where it includes the optical filter according to the embodiment of the present invention.

As another configuration of the self-luminous display device according to the embodiment of the present invention, it is possible to use a configuration of a generally used self-luminous display device, for example, a micro light emitting diode (micro LED) display device or a mini light emitting diode (mini LED) display device without particular limitation, as long as the optical filter according to the embodiment of the present invention is included. The configuration example of the self-luminous display device according to the embodiment of the present invention is not particularly limited, and examples thereof include a display device including glass, a layer containing a thin film transistor (TFT), a light emitting element, the optical filter according to the embodiment of the present invention, and a surface film, in order from the opposite side to external light.

As a light source of the display light of the self-luminous display device according to the embodiment of the present invention, a single blue color may be used, or the three primary colors of blue, green, and red may be used, as long as the light emitting diode is provided as a light emitting source. For example, it is also preferable to use a combination of a blue light source that emits light in a wavelength range of 440 nm to 470 nm, a green light source that emits light in a wavelength range of 520 nm to 560 nm, and a red light source that emits light in a wavelength range of 620 nm to 660 nm.

In the present invention, the mini LED means an LED having a chip size of about 100 to 200 μm, and the micro LED means an LED having a chip size of less than 100 μm. Preferred examples of the micro LED include the micro LED described in WO2014/204694A.

In the self-luminous display device according to the embodiment of the present invention, it is preferable that the optical filter according to the embodiment of the present invention is bonded to the glass (the base material) while interposing a pressure sensitive adhesive layer, on a surface positioned opposite to the side of the external light.

For the pressure sensitive adhesive layer, regarding the description related to the pressure sensitive adhesive layer and the forming method in the self-luminous display device described in [0272] to [0298] of WO2021/221122A, the wavelength selective absorption layer according to the embodiment of the present invention can be read as the optical filter according to the embodiment of the present invention and applied as it is.

<Ultraviolet Absorbing Layer>

A self-luminous display device such as an organic electroluminescent display device, light emitting diodes (micro LED) display device, or a mini LED display device, or a liquid crystal display device, which includes the optical filter according to the embodiment of the present invention, preferably has a layer (hereinafter, also referred to as an “ultraviolet absorbing layer”), which inhibits the light absorption (ultraviolet absorption) of the compound that generates a radical upon ultraviolet irradiation or light absorption of the partial structure that generates a radical upon ultraviolet irradiation, is provided on a viewer side with respect to the optical filter according to the embodiment of the present invention. In a case where the ultraviolet absorbing layer is provided, it is possible to prevent the fading of the optical filter according to the embodiment of the present invention due to external light.

The ultraviolet absorbing layer according to the embodiment of the present invention will be described below.

(Ultraviolet Absorbing Agent)

The ultraviolet absorbing layer according to the embodiment of the present invention contains a resin and an ultraviolet absorbing agent. From the viewpoint of the excellent absorption capacity of an ultraviolet ray having a wavelength of 370 nm or less and good liquid crystal display properties, an ultraviolet absorbing agent having a small absorption of visible light having a wavelength of 400 nm or more is preferably used.

Specific examples of the ultraviolet absorbing agent preferably used in the present invention include a hindered phenol-based compound, a hydroxybenzophenone-based compound, a benzotriazole-based compound, a salicylic acid ester-based compound, a benzophenone-based compound, a cyanoacrylate-based compound, and a nickel complex salt-based compound.

For the specific examples of the hindered phenol-based compound and the benzotriazole-based compound, it is possible to apply, for example, the descriptions related to the specific examples of the hindered phenol-based compound and the benzotriazole-based compound as they are, which are described in [0313] of WO2021/132674A.

The adding amount of this ultraviolet absorbing agent is preferably 0.1 part by mass to 30.0 parts by mass with respect to 100 parts by mass of the resin.

(Resin)

In the present invention, as the resin that is used for the ultraviolet absorbing layer, a known resin can be used, which is not particularly limited as long as it does not contradict the gist of the present invention. Examples of the resin include a cellulose acylate resin, an acrylic resin, a cycloolefin-based resin, a polyester-based resin, and an epoxy resin.

(Installation Position of Ultraviolet Absorbing Layer)

In the present invention, the disposition of the ultraviolet absorbing layer is not particularly limited as long as it is on the viewer side with respect to the optical filter according to the embodiment of the present invention, and the ultraviolet absorbing layer can be installed at any position. For example, it is also possible to add an ultraviolet absorbing agent to a member such as a protective film of the polarizing plate, an antireflection film, or the like to impart it a function of an ultraviolet absorbing layer.

### Examples

Hereinafter, the present invention will be described in more detail based on Examples. The materials, using amount, ratio, details of treatment, procedures of treatment, and the like described in Examples below can be appropriately changed without departing from the spirit of the present invention. Therefore, it is to be understood that the scope of the present invention is not limited to Examples described below.

It is noted that “parts” and “%” that indicate the composition in Examples below are based on mass unless otherwise specified. Room temperature means “25° C.”.

All steps from a preparation step of a light absorption filter forming liquid to a production step of a base material-attached light absorption filter using the light absorption filter forming liquid and to the use thereof in the ultraviolet irradiation test were carried out under a yellow lamp to avoid ultraviolet irradiation.

[Production of Light Absorption Filter]

Materials used to produce the light absorption filter are shown below. It is noted that the content in the copolymer is a content based on parts by weight.

<Matrix Polymer>

[Polymer Having Both High Affinity Part and Low Affinity Part]

(Resin 1)

Hydrogenated styrene-diene block copolymer (manufactured by Asahi Kasei Corporation, TUFTEC H1043 (product name)), styrene content: 67%.

(Resin 2)

Hydrogenated styrene-diene block copolymer (manufactured by Asahi Kasei Corporation, TUFTEC H1517 (product name)), styrene content: 43%.

(Resin 3)

Maleic acid anhydride-modified hydrogenated styrene-diene block copolymer (manufactured by Asahi Kasei Corporation, TUFTEC M1913 (product name)), styrene content: 30%

(Resin 4)

Maleic acid anhydride-modified hydrogenated styrene-diene block copolymer (manufactured by Asahi Kasei Corporation, TUFTEC M1943 (product name)), styrene content: 20%

(Resin 5)

Partially hydrogenated styrene-diene block copolymer (manufactured by Asahi Kasei Corporation, TUFTEC P2000 (product name)), styrene content: 67%

(Resin 6)

Partially hydrogenated styrene-diene block copolymer (manufactured by Asahi Kasei Corporation, TUFTEC P5051 (product name)), styrene content: 47%

[Polymer Including Partial Structure that Generates Radical Upon Ultraviolet Irradiation]

(Resin 8)

Benzyl methacrylate-methacrylic acid-based copolymer (manufactured by Fujikura Kasei Co., Ltd., ACRYBASE FF-187 (product name)), benzyl methacrylate ratio: 87% (benzyl methacrylate content: 87%)

(Resin 9)

Polybenzyl methacrylate (manufactured by Sigma-Aldrich Co., LLC, poly(benzyl methacrylate))

(Resin 11)

Polymethyl methacrylate (manufactured by Mitsubishi Chemical Corporation, DIANAL BR80 (product name))

[Comparative Polymer]

(Resin 7)

Polystyrene resin (PSJ-polystyrene GPPS SGP-10 (product name), manufactured by PS Japan Corporation).

(Resin 10)

Cyclic polyolefin resin (APL6509T (product name), manufactured by Mitsui Chemicals, Inc., a copolymer of ethylene and norbornene, Tg: 80° C.)

<Dye>

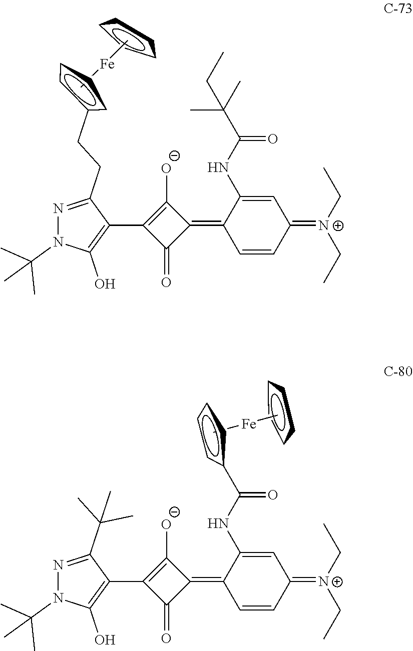

Solvent Blue 35: 1,4-bis(butylamino)-9,10-anthraquinone, an anthraquinone-based coloring agent

(Leveling agent 1)

A polymer surfactant composed of the following constitutional components was used as a leveling agent 1. In the following structural formulae, the proportion of each constitutional component is in terms of a molar ratio, and t-Bu means a tert-butyl group.

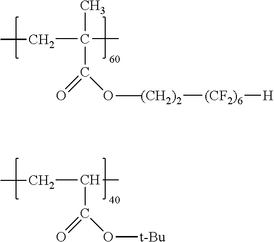

(Base Material 1)

Cellulose acylate film (manufactured by FUJIFILM Corporation, ZRG40UL (product name))

(Base material 2) Polyethylene terephthalate film (manufactured by TORAY INDUSTRIES, Inc., Lumirror XD-510P (product name, film thickness: 50 μm))

### Examples

<1. Production of Base Material-Attached Light Absorption Filter No. 101>

(1) Preparation of Resin Solution (Light Absorption Filter Forming Liquid)

Each component was mixed with the composition shown below to prepare a light absorption filter forming liquid (composition) Ba-1.

Composition of light absorption filter forming liquid Ba-1

| | | |
|---|---|---|
| Resin 1 | 96.9 | parts by mass |
| Leveling agent 1 | 0.16 | parts by mass |
| Dye C-73 | 1.57 | parts by mass |
| Photoradical generator: | 1.37 | parts by mass |
| 4,4′-dimethoxybenzophenone | | |
| (manufactured by Tokyo | | |
| Chemical Industry Co., Ltd.) | | |
| Cyclohexane (solvent) | 748.0 | parts by mass |
| Ethyl acetate (solvent) | 132.0 | parts by mass |

Subsequently, the obtained light absorption filter forming liquid Ba-1 was filtered using a filter paper (#63, manufactured by Toyo Filter Paper Co., Ltd.) having an absolute filtration precision of 10 μm, and further subjected to filtration using a metal sintered filter (product name: Pall filter PMF, media code: FH025, manufactured by Pall) with an absolute filtration precision of 2.5 μm.

(2) Production of Base Material-Attached Light Absorption Filter

The light absorption filter forming liquid Ba-1 after the filtration treatment was applied onto a base material 1 by using a bar coater so that the film thickness after drying was 2.5 μm, and dried at 120° C. to produce a base material-attached light absorption filter No. 101.

<2. Production of Base Material-Attached Light Absorption Filter Nos. 102 to 106 and 201 to 208>

Base material-attached light absorption filter Nos. 102 to 106 and 201 to 208 were produced in the same manner as in the production of the base material-attached light absorption filter No. 101, except that the kind of the matrix polymer, the presence or absence of the formulation of the dye, or the presence or absence of the radical generator was changed to the contents shown in Table 1.

<3. Production of Base Material-Attached Light Absorption Filter No. 107>

(1) Preparation of Resin Solution (Light Absorption Filter Forming Liquid)

Each component was mixed with the composition shown below to prepare a light absorption filter forming liquid (composition) Ba-7.

Composition of light absorption filter forming liquid Ba-7

| | | |
|---|---|---|
| Resin 8 | 98.4 | parts by mass |
| Leveling agent 1 | 0.08 | parts by mass |
| Dye C-73 | 1.57 | parts by mass |
| Methyl ethyl ketone (solvent) | 566.7 | parts by mass |

Subsequently, the obtained light absorption filter forming liquid Ba-7 was filtered using a filter paper (#63, manufactured by Toyo Filter Paper Co., Ltd.) having an absolute filtration precision of 10 μm, and further subjected to filtration using a metal sintered filter (product name: Pall filter PMF, media code: FH025, manufactured by Pall) with an absolute filtration precision of 2.5 km.

(2) Production of Base Material-Attached Light Absorption Filter

The light absorption filter forming liquid Ba-7 after the filtration treatment was applied onto a base material 2 by using a bar coater so that the film thickness after drying was 2.5 μm, and dried at 120° C. to produce a base material-attached light absorption filter No. 107.

<4. Production of Base Material-Attached Light Absorption Filter Nos. 108 to 111, 209, 210, and 213>

Base material-attached light absorption filter Nos. 108 to 111, 209, 210, and 213 were produced in the same manner as in the production of the base material-attached light absorption filter No. 107, except that the kind of the matrix polymer, the kind and formulation amount of the dye, or the presence or absence of the radical generator was changed to the contents shown in Table 1.

<5. Production of Base Material-Attached Light Absorption Filter No. 211>

(1) Preparation of Resin Solution (Light Absorption Filter Forming Liquid)

Each component was mixed with the composition shown below to prepare a light absorption filter forming liquid (composition) Ba-11.

Composition of light absorption filter forming liquid Ba-11

| | | |
|---|---|---|
| Resin 10 | 93.5 | parts by mass |
| Dye C-73 | 1.57 | parts by mass |
| Photoradical generator: | 1.37 | parts by mass |
| 4,4′-dimethoxybenzophenone | | |
| (manufactured by Tokyo | | |
| Chemical Industry Co., Ltd.) | | |
| Peelability control resin | 3.4 | parts by mass |
| component: TUFTEC H-1043 | | |
| (product name, manufactured | | |
| by Asahi Kasei Corporation) | | |
| Leveling agent: MEGAFACE | 0.16 | parts by mass |
| F-554 (product name, manufactured | | |
| by DIC Corporation, fluoropolymer) | | |
| Cyclohexane (solvent) | 770.0 | parts by mass |

Subsequently, the obtained light absorption filter forming liquid Ba-11 was filtered using a filter paper (#63, manufactured by Toyo Filter Paper Co., Ltd.) having an absolute filtration precision of 10 μm, and further subjected to filtration using a metal sintered filter (product name: Pall filter PMF, media code: FH025, manufactured by Pall) with an absolute filtration precision of 2.5 μm.

(2) Production of Base Material-Attached Light Absorption Filter

The light absorption filter forming liquid Ba-11 after the filtration treatment was applied onto a base material 1 by using a bar coater so that the film thickness after drying was 2.5 μm, and dried at 120° C. to produce a base material-attached light absorption filter No. 211.

<6. Production of Base Material-Attached Light Absorption Filter Nos. 212, 214, and 215>

Base material-attached light absorption filter Nos. 212, 214, and 215 were produced in the same manner as in the production of the base material-attached light absorption filter No. 211, except that the kind and formulation amount of the dye, the kind and formulation amount of the radical generator were changed to the contents shown in Table 1.

[Production of Light Absorption Filter Having Gas Barrier Layer]

Regarding each of the base material-attached light absorption filter Nos. 101 to 111 and 201 to 213, a light absorption filter (a light absorption filter having a gas barrier layer) formed by further laminating a gas barrier layer on the light absorption filter was produced as described below, and the evaluation described later was carried out.

(1) Production of Base Material 3

The light absorption filter side of the base material-attached light absorption filter was subjected to a corona treatment using a corona treatment device (product name: Corona-Plus, manufactured by VETAPHONE) under the conditions of a discharge amount of 1,000 W min/m² and a processing speed of 3.2 m/min and used as a base material 3.

(2) Preparation of Resin Solution

Each component was mixed with the composition shown below, and the resultant mixture was stirred in a constant-temperature tank at 90° C. for 1 hour to dissolve Kuraray Exceval AQ-4105 (product name, manufactured by KURARAY Co., Ltd., modified polyvinyl alcohol, saponification degree: 98% to 99% by mole), whereby a gas barrier layer forming liquid was prepared.

Composition of gas barrier layer forming liquid

| | | |
|---|---|---|
| Kuraray Exceval AQ-4105 | 4.0 | parts by mass |
| (product name, manufactured | | |
| by KURARAY Co., Ltd.) | | |
| Pure water | 88.5 | parts by mass |
| Isopropyl alcohol | 7.5 | parts by mass |

Subsequently, the obtained gas barrier layer forming liquid was filtered using a filter having an absolute filtration precision of 5 μm (product name: Hydrophobic Fluorepore Membrane, manufactured by Millex).

(3) Lamination of Gas Barrier Layer

The gas barrier layer forming liquid after the filtration treatment was applied to the corona-treated surface side of the base material 3 using a bar coater so that the film thickness after drying was 1.6 μm, and dried at 120° C. for 60 seconds, whereby a light absorption filter having a gas barrier layer was produced.

The light absorption filter having a gas barrier layer has a configuration in which the base material 1 or base material 2, the light absorption filter, and the gas barrier layer are laminated in this order.

<Absorbance of Light Absorption Filter (Before Ultraviolet Irradiation)>

(1) Measurement of Absorbance

Using a UV3600 spectrophotometer (product name) manufactured by Shimadzu Corporation, the absorbance of the light absorption filter having a gas barrier layer and the standard filter in a wavelength range of 380 to 800 nm was measured for every 1 nm. It is noted that the optical path length is 2.5 μm.

Here, Nos. 101 to 111 are the light absorption filters according to the embodiment of the present invention, Nos. 208, 211, 214, and 215 are light absorption filters for comparison, and Nos. 201 to 207, 209, 210, 212, and 213 are light absorption filters for reference.

A standard filter for the light absorption filter No. 101 containing the resin 1 is the light absorption filter No. 201 which was changed not to contain a dye and a photoradical generator.

A standard filter for the light absorption filter No. 102 containing the resin 2 is the light absorption filter No. 202 which was changed not to contain a dye and a photoradical generator.

A standard filter for the light absorption filter No. 103 containing the resin 3 is the light absorption filter No. 203 which was changed not to contain a dye and a photoradical generator.

A standard filter for the light absorption filter No. 104 containing the resin 4 is the light absorption filter No. 204 which was changed not to contain a dye and a photoradical generator.

A standard filter for the light absorption filter No. 105 containing the resin 5 is the light absorption filter No. 205 which was changed not to contain a dye and a photoradical generator.

A standard filter for the light absorption filter No. 106 containing the resin 6 is the light absorption filter No. 206 which was changed not to contain a dye and a photoradical generator.

A standard filter for the light absorption filter No. 208 containing the resin 7 is the light absorption filter No. 207 which was changed not to contain a dye and a photoradical generator.

A standard filter for the light absorption filter Nos. 107 and 109 containing the resin 8 is the light absorption filter No. 209 which was changed not to contain a dye.

A standard filter for the light absorption filter Nos. 108 and 110 containing the resin 9 is the light absorption filter No. 210 which was changed not to contain a dye.

A standard filter for the light absorption filter Nos. 211, 214, and 215 containing the resin 10 is the light absorption filter No. 212 which was changed not to contain a dye.

A standard filter for the light absorption filter Nos. 111 containing the resin 11 is the light absorption filter No. 213 which was changed not to contain a dye.

(2) Calculation of Absorbance

Using the absorbance value Abₓ (λ) of the light absorption filter having a gas barrier layer at each wavelength λ nm measured as described above and the absorbance value Ab₀ (λ) of the standard filter containing the same resin at each wavelength λ nm, the absorbance Ab (λ) of the light absorption filter before ultraviolet irradiation was calculated according to the following expression.

Ab(λ)=Abₓ(λ)−Ab₀(λ)

Hereinafter, among the absorbances Ab (λ) of the light absorption filter in a wavelength range of 400 to 700 nm, the wavelength at which the highest absorbance Ab (λ) among the wavelengths at which the highest maximal absorption is exhibited was defined as the maximal absorption wavelength (hereinafter, also simply referred to as “λₘₐₓ”), and the absorbance at λₘₐₓ was defined as the absorption maximum value (hereinafter, also simply referred to as “Ab (λₘₐₓ)”).

<<Evaluation 1>>

Each light absorption filter was subjected to the following ultraviolet irradiation test to evaluate the decolorization rate and the presence or absence of the secondary absorption associated with the decomposition of the coloring agent.

The results are summarized in Table 2 below.

(Ultraviolet Irradiation Test 1)

Using an air-cooled metal halide lamp (manufactured by EYE GRAPHICS Co., Ltd.) at 160 W/cm under atmospheric pressure (101.33 kPa), the light absorption filter having a gas barrier layer and the standard filter were irradiated at room temperature with an ultraviolet ray at an irradiation dose of 500 mJ/cm² from the light absorption filter side (the side opposite to base material 1 or the base material 2).

<Absorbance of Light Absorption Filter (after Ultraviolet Irradiation)>

Using the light absorption filter having a gas barrier layer after ultraviolet irradiation and the standard filter, the absorbance Ab (k) of the light absorption filter after ultraviolet irradiation was calculated according to the same method as described in <Absorbance of light absorption filter (before ultraviolet irradiation)> described above.

[1. Evaluation of Decolorization Rate]

The decolorization rate was calculated according to the following expression using the absorption maximum values (Ab (λₘₐₓ)) before and after the ultraviolet irradiation test.

Decolorization rate (%)=100−(Ab(λₘₐₓ) after ultraviolet irradiation/Ab(λₘₐₓ)before ultraviolet irradiation)×100

[2. Evaluation of Presence or Absence of Secondary Absorption Associated with Decomposition of Coloring Agent]

The presence or absence of the absorption (the secondary absorption) derived from a new coloration structure associated with the decomposition of the coloring agent was evaluated based on the ratio of the absorbance at a wavelength of 450 nm to the absorption maximum value (Ab (λₘₐₓ)) before ultraviolet irradiation (hereinafter, also simply referred to as “Ab (450)”). It is meant that the smaller the value obtained by subtracting the ratio of the following (I) from the ratio of the following (II), the less frequently the absorption derived from the new coloration structure associated with the decomposition of the coloring agent occurs.
(I) Ab (450) before ultraviolet irradiation/Ab (λₘₐₓ)×100% before ultraviolet irradiation
(II) Ab (450) after ultraviolet irradiation/Ab (λₘₐₓ) before ultraviolet irradiation×100%

TABLE 1

| | | | | | Radical generator | | |
|---|---|---|---|---|---|---|---|
| | | Dye | | | | | Molar |
| | Matrix | | Amax | Formulation | | Formulation | ratio |
| No. | polymer | Kind | (nm) | amount | Kind | amount | to dye |
| 101 | Resin 1 | C-73 | 592 | 1.57 | 4.4′- | 1.37 | 2.5 |
| | | | | | dimethoxybenzophenone | | |
| 102 | Resin 2 | C-73 | 592 | 1.57 | 4.4′- | 1.37 | 2.5 |
| | | | | | dimethoxybenzophenone | | |
| 103 | Resin 3 | C-73 | 590 | 1.57 | 4.4′- | 1.37 | 2.5 |
| | | | | | dimethoxybenzophenone | | |
| 104 | Resin 4 | C-73 | 590 | 1.57 | 4.4′- | 1.37 | 2.5 |
| | | | | | dimethoxybenzophenone | | |
| 105 | Resin 5 | C-73 | 590 | 1.57 | 4.4′- | 1.37 | 2.5 |
| | | | | | dimethoxybenzophenone | | |
| 106 | Resin 6 | C-73 | 590 | 1.57 | 4.4′- | 1.37 | 2.5 |
| | | | | | dimethoxybenzophenone | | |
| 107 | Resin 8 | C-73 | 594 | 1.57 | Absent | — | — |
| 108 | Resin 9 | C-73 | 595 | 1.57 | Absent | — | — |
| 109 | Resin 8 | C-80 | 601 | 1.47 | Absent | — | — |
| 110 | Resin 9 | C-80 | 601 | 1.47 | Absent | — | — |
| 111 | Resin | C-73 | 588 | 1.57 | Absent | — | — |
| | 11 | | | | | | |
| 201 | Resin 1 | Absent | — | — | Absent | — | — |
| 202 | Resin 2 | Absent | — | — | Absent | — | — |
| 203 | Resin 3 | Absent | — | — | Absent | — | — |
| 204 | Resin 4 | Absent | — | — | Absent | — | — |
| 205 | Resin 5 | Absent | — | — | Absent | — | — |
| 206 | Resin 6 | Absent | — | — | Absent | — | — |
| 207 | Resin 7 | Absent | — | — | Absent | — | — |
| 208 | Resin 7 | C-73 | 590 | 1.57 | 4.4′- | 1.37 | 2.5 |
| | | | | | dimethoxybenzophenone | | |
| 209 | Resin 8 | Absent | — | — | Absent | — | — |
| 210 | Resin 9 | Absent | — | — | Absent | — | — |
| 211 | Resin | C-73 | 590 | 1.57 | 4.4′- | 1.37 | 2.5 |
| | 10 | | | | dimethoxybenzophenone | | |
| 212 | Resin | Absent | — | — | Absent | — | — |
| | 10 | | | | | | |
| 213 | Resin | Absent | — | — | Absent | — | — |
| | 11 | | | | | | |
| 214 | Resin | Solvent | 651 | 3.97 | Benzophenone | 6.3 | 15 |
| | 10 | Blue | | | | | |
| | | 35 | | | | | |
| 215 | Resin | Solvent | 651 | 3.97 | Benzophenone*¹ | 6.3 | 5 |
| | 10 | Blue | | | | | |
| | | 35 | | | | | |

It is noted that regarding the light absorption filter Nos. 101 to 106 in which a polymer having both a high affinity part and a low affinity part has been used, and the light absorption filter Nos. 208, 211, 214, and 215, in which a comparative polymer having only one of a high affinity part or a low affinity part has been used, each of the differences between the fd, fp, and fh of each of the high affinity part and the low affinity part and the fd, fp, and fh of each of the dye and the radical generator, which are calculated based on the above-described expression is shown in Table A below.

TABLE A

| | High affinity part | | | | | | Low affinity part | | | | | |
|---|---|---|---|---|---|---|---|---|---|---|---|---|
| | Difference | | | Difference from radical | | | Difference from | | | Difference from | | |
| | from dye | | | generator | | | dye | | | radical generator | | |
| No. | fp | fh | fd | fp | fh | fd | fp | fh | fd | fp | fh | fd |
| 101 to | −7 | −6 | 13 | −6 | −6 | 12 | −33 | −23 | 56 | −33 | −22 | 55 |
| 106 | | | | | | | | | | | | |
| 208 | −7 | −6 | 13 | −6 | −6 | 12 | Not containing low affinity part | | | | | |
| 211 | Not containing high affinity part | | | | | | −14 | −5 | 19 | −13 | −5 | 18 |
| 214 to | Not containing high affinity part | | | | | | −12 | −7 | 19 | −16 | 3 | 13 |
| 215 | | | | | | | | | | | | |

It is noted that regarding the dye C-73, the values of fp, fh, and fd are calculated based on the structure in which the ferrocenyl group is replaced with a hydrogen atom in the following resonance structure.

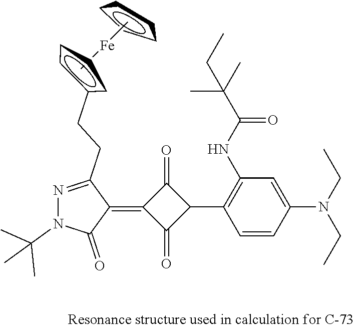

TABLE 2

| | | | | | | Ratio of Ab (450) to Ab | |
|---|---|---|---|---|---|---|---|
| | | | | | | (λmax) before | |
| | Ab (λmax) of dye | | | Ab (450) | | ultraviolet irradiation | |
| | Before | After | | Before | After | Before | After |
| | ultraviolet | ultraviolet | Decolorization | ultraviolet | ultraviolet | ultraviolet | ultraviolet |
| No. | irradiation | irradiation | rate | irradiation | irradiation | irradiation | irradiation |
| 101 | 0.97 | 0.48 | 51% | 0.02 | 0.02 | 2.3% | 2.3% |
| 102 | 0.91 | 0.12 | 87% | 0.02 | 0.02 | 2.4% | 2.4% |
| 103 | 1.00 | 0.02 | 98% | 0.02 | 0.02 | 1.6% | 1.6% |
| 104 | 0.94 | 0.02 | 98% | 0.02 | 0.02 | 1.9% | 1.9% |
| 105 | 1.16 | 0.64 | 45% | 0.02 | 0.02 | 1.8% | 1.8% |
| 106 | 0.73 | 0.10 | 86% | 0.02 | 0.02 | 2.2% | 2.2% |
| 107 | 1.01 | 0.01 | 99% | 0.02 | 0.02 | 2.0% | 2.0% |
| 108 | 1.02 | 0.01 | 99% | 0.02 | 0.02 | 2.0% | 2.0% |
| 109 | 0.85 | 0.01 | 99% | 0.02 | 0.02 | 2.4% | 2.4% |
| 110 | 0.86 | 0.01 | 99% | 0.02 | 0.02 | 2.3% | 2.3% |
| 111 | 0.90 | 0.40 | 56% | 0.02 | 0.02 | 2.2% | 2.2% |
| 208 | 1.00 | 0.70 | 30% | 0.02 | 0.02 | 2.0% | 2.0% |
| 211 | 0.98 | 0.68 | 31% | 0.02 | 0.02 | 2.0% | 2.0% |
| 214 | 0.105 | 0.100 | 5% | 0.000 | 0.01 | 0% | 9.5% |
| 215 | 0.102 | 0.100 | 2% | 0.000 | 0.01 | 0% | 9.8% |

(Notes in Tables 1, 2, and A)

Nos. 101 to 111 and 201 to 215 mean the light absorption filter Nos. 101 to 111 and 201 to 215, respectively.

The molar ratio to the dye means the molar amount of the formulated radical generator with respect to 1 mol of the dye.

*1 in the light absorption filter No. 215 means that in addition to 6.3 parts by mass of benzophenone which is a radical generator, the light absorption filter No. 215 contains 6.7 parts by mass of ethyl 4-(dimethylamino)benzoate (15 mol in terms of the formulation ratio with respect to 1 mol of the dye) as a decolorization accelerating agent.

λₘₐₓ means a wavelength at which the highest absorbance Ab (λ) is exhibited among the maximal absorption wavelengths that the light absorption filter has in a wavelength range of 400 to 700 nm.

The formulation amount of the dye means an amount in terms of a part by mass with respect to 100 parts by mass of the filter. The formulation amount of the radical generator means an amount in terms of a part by mass with respect to 100 parts by mass of the filter. It is noted that the content of the resin is adjusted so that the above-described formulation amounts of the dye and the radical generator are obtained.

Ab (λₘₐₓ) means the value of the absorbance at the maximal absorption wavelength λₘₐₓ.

Ab (450) means a value of absorbance at a wavelength of 450 nm.

Regarding the ratio (%) of Ab (450) to Ab (λₘₐₓ) before ultraviolet irradiation, the column of “Before ultraviolet irradiation” means a ratio calculated by using the Ab (450) before ultraviolet irradiation, and the column of “After ultraviolet irradiation” means a ratio calculated by using the Ab (450) after ultraviolet irradiation.

From the results in Table 1 and Table 2, the following points can be seen.

The light absorption filter No. 208 of Comparative Example contains a dye, a radical generator, and a polystyrene consisting of only a high affinity part with respect to the dye and the radical generator. In addition, the light absorption filter No. 211 of Comparative Example contains a dye, a radical generator, and a copolymer of ethylene and norbornene, consisting of only a low affinity part with respect to the dye and the radical generator. In these light absorption filter Nos. 208 and 211 of Comparative Examples, the decolorization rate upon ultraviolet irradiation was as low as 30% or 31%.

In addition, the light absorption filter Nos. 214 and 215 of Comparative Example contain a dye, a radical generator, and a copolymer of ethylene and norbornene, consisting of only a low affinity part with respect to the dye and the radical generator. The light absorption filters No. 214 and 215 of these comparative examples seldom decolorized and have a decolorization rate of 5% and 2%, respectively, upon ultraviolet irradiation, and moreover, Ab (450)/Ab (λₘₐₓ) increases from 0% to 9.5% and from 0% to 9.8%, respectively, upon ultraviolet irradiation, whereby it has been found that the absorption derived from a new coloration structure associated with the decomposition of the coloring agent occurs.

On the other hand, in all of Nos. 101 to 106, which contain the matrix polymer containing both a high affinity part and a low affinity part with respect to a dye and a radical generator, and Nos. 107 to 111, which contain the resin composed of a polymer including a partial structure that generates a radical upon ultraviolet irradiation, the decolorization rate upon ultraviolet irradiation is high with respect to the light absorption filter of Comparative Example, the decolorizing property is excellent, and the secondary absorption associated with the dye decomposition upon ultraviolet irradiation hardly occurred.

<7. Production of Base Material-Attached Light Absorption Filter No. 301>

(1) Preparation of Resin Solution (Light Absorption Filter Forming Liquid)

Each component was mixed with the composition shown below to prepare a light absorption filter forming liquid (composition) Ba-3.

Composition of light absorption filter forming liquid Ba-3

| | | |
|---|---|---|
| Resin 8 | 78.4 | parts by mass |
| Leveling agent 1 | 0.08 | parts by mass |
| Dye C-73 | 1.57 | parts by mass |
| Photoradical generator: | 20.0 | parts by mass |
| Irgacure 2959 | | |
| (manufactured by BASF SE) | | |
| Methyl ethyl ketone (solvent) | 567.0 | parts by mass |

Subsequently, the obtained light absorption filter forming liquid Ba-3 was filtered using a filter paper (#63, manufactured by Toyo Filter Paper Co., Ltd.) having an absolute filtration precision of 10 μm, and further subjected to filtration using a metal sintered filter (product name: Pall filter PMF, media code: FH025, manufactured by Pall) with an absolute filtration precision of 2.5 μm.

(2) Production of Base Material-Attached Light Absorption Filter

The light absorption filter forming liquid Ba-3 after the filtration treatment was applied onto a base material 1 by using a bar coater so that the film thickness after drying was 2.5 μm, and dried at 120° C. to produce a base material-attached light absorption filter No. 301 according to the embodiment of the present invention.

[Production of Light Absorption Filter Having Gas Barrier Layer]

A light absorption filter (a light absorption filter having a gas barrier layer) formed by further laminating a gas barrier layer on the base material-attached light absorption filter No. 301 was produced in the same manner as in the base material-attached light absorption filter No. 101, and the following evaluation was carried out.

<<Evaluation 2>>

Regarding the light absorption filter No. 301 having a gas barrier layer was subjected to the following ultraviolet irradiation test, and the decolorization rate and the reproducibility of the mask pattern were evaluated.

The results are summarized in Table 3 below.

(Ultraviolet Irradiation Test 2)

Quartz glass having a thickness of 2.5 mm was covered on a gas barrier layer of the light absorption filter No. 301 having the gas barrier layer, and using an ultra-high pressure mercury lamp (manufactured by HOYA Corporation) under atmospheric pressure (101.33 kPa), ultraviolet rays (UV) having the illuminance and the irradiation dose shown in Table 3 were applied at room temperature from the light absorption filter side (the side opposite to the base material 1), and then the decolorization rate was calculated in the same manner as in the evaluation 1.

(Ultraviolet Irradiation Test 3)

A mask manufactured by TOPIX corporation (a mask obtained by subjecting, to vapor deposition on quartz glass having a thickness of 2.3 mm, a striped mask made of chrome having a pitch of a light transmission part of 90 μm and a pitch of a light non-transmission part of 180 μm) was covered on the light absorption filter No. 301 having a gas barrier layer, and using an ultra-high pressure mercury lamp (manufactured by HOYA Corporation) under atmospheric pressure (101.33 kPa), ultraviolet rays (UV) having the illuminance and the irradiation dose shown in Table 3 were applied at room temperature from the light absorption filter side (the side opposite to the base material 1). The pattern (the width of the light transmission part) formed in the sample after the ultraviolet irradiation was observed with an optical microscope, and the pattern reproducibility was evaluated according to the following standard. The closer the ratio in the following evaluation standard is to 100%, the better the pattern reproducibility is.

—Evaluation Standard—

A: The ratio of the width of the light transmission part within the area of 40 mm×40 mm to the width of the original mask is more than 90% and less than 110%.

B: The ratio of the width of the light transmission part within the area of 40 mm×40 mm to the width of the original mask is more than 60% and 90% or less, or 110% or more and less than 140%.

C: The ratio of the width of the light transmission part within the area of 40 mm×40 mm to the width of the original mask is 60% or less, or 140% or more.

TABLE 3

| | | Distance | | | | |
|---|---|---|---|---|---|---|
| | | between | | | | |
| | | UV lamp | | | | |
| | Light | and light | | Irradiation | | |
| | absorption | absorption | Illuminance | dose | Decolorization | Pattern |
| | filter | filter (cm) | (mW/cm²) | (mJ/cm²) | rate | reproducibility |
| 301- | 301 | 30 | 100 | 2000 | 98% | B |
| A | | | | | | |
| 301- | 301 | 80 | 10 | 2000 | 78% | A |
| B | | | | | | |

From the results of Table 3, it can be seen that the light absorption filter No. 301 according to the embodiment of the present invention exhibits an excellent decolorization rate and can faithfully reproduce the mask pattern, which is preferable. In particular, under the conditions in which the distance between the ultraviolet lamp and the light absorption filter is widened to carry out exposure with low illuminance, the reproducibility of the mask pattern is higher, which is preferable.

Although the present invention has been described with reference to the embodiments, it is the intention of the inventors of the present invention that the present invention should not be limited by any of the details of the description unless otherwise specified and rather be construed broadly within the spirit and scope of the invention appended in WHAT IS CLAIMED IS.

The present application claims priority based on JP2021-001136 filed on Jan. 6, 2021 in Japan, JP2021-057023 filed on Mar. 30, 2021 in Japan, JP2021-116334 filed on Jul. 14, 2021 in Japan, and JP2021-208371 filed on Dec. 22, 2021 in Japan, the contents of which are incorporated herein as a part of the present specification by reference.

### EXPLANATION OF REFERENCES

1: upper polarizing plate
2: direction of absorption axis of upper polarizing plate
3: liquid crystal cell upper electrode substrate
4: alignment control direction of upper substrate
5: liquid crystal layer
6: liquid crystal cell lower electrode substrate
7: alignment control direction of lower substrate
8: lower polarizing plate
9: direction of absorption axis of lower polarizing plate
B: backlight unit
10: liquid crystal display device

What is claimed is:

1. A light absorption filter comprising:
a resin;
a dye that has a main absorption wavelength band in a wavelength range of 400 to 700 nm; and
a compound that generates a radical upon ultraviolet irradiation,
wherein the resin is composed of a polymer having both a partial structure having a high affinity and a partial structure having a low affinity, with respect to the dye and the compound that generates a radical upon ultraviolet irradiation.

2. The light absorption filter according to claim 1,
wherein the dye includes a squaraine-based coloring agent represented by General Formula (1), 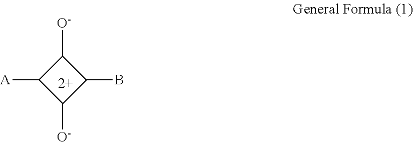
in the formula, A and B each independently represent an aryl group which may have a substituent, a heterocyclic group which may have a substituent, or —CH=G,
where G represents a heterocyclic group which may have a substituent.

3. The light absorption filter according to claim 1,
wherein the polymer constituting the resin is a block copolymer.

4. The light absorption filter according to claim 1,
wherein the partial structure having a high affinity includes a polystyrene structure.

5. The light absorption filter according to claim 1,
wherein the partial structure having a low affinity includes a structure obtained by hydrogenating polydiene.

6. The light absorption filter according to claim 1,
wherein the compound that generates a radical upon ultraviolet irradiation is a compound that generates a radical upon intramolecular cleavage, and
the partial structure that generates a radical upon ultraviolet irradiation is a partial structure that generates a radical upon intramolecular cleavage.

7. The light absorption filter according to claim 1,
wherein the compound that generates a radical upon ultraviolet irradiation is a compound that generates a radical by extracting a hydrogen atom from a compound that is present in a vicinity of the compound, and
the partial structure that generates a radical upon ultraviolet irradiation is a partial structure that generates a radical by extracting a hydrogen atom from a compound that is present in a vicinity of the partial structure.

8. The light absorption filter according to claim 7,
wherein the compound that generates a radical by extracting a hydrogen atom from a compound that is present in a vicinity of the compound is a benzophenone compound substituted with an alkoxy group, and
the partial structure that generates a radical by extracting a hydrogen atom from a compound that is present in a vicinity of the partial structure is a partial structure derived from a benzophenone compound substituted with an alkoxy group.

9. The light absorption filter according to claim 1,
wherein in the light absorption filter, the dye is chemically changed to be decolorized upon irradiation with light.

10. An optical filter that is obtained by subjecting the light absorption filter according to claim 1 to mask exposure by ultraviolet irradiation.

11. A self-luminous display device, an organic electroluminescent display device, or a liquid crystal display device, comprising the optical filter according to claim 10.

12. The self-luminous display device, the organic electroluminescent display device, or the liquid crystal display device, according to claim 11,
wherein a layer, which inhibits light absorption of the compound that generates a radical upon ultraviolet irradiation or light absorption of the partial structure that generates a radical upon ultraviolet irradiation, is provided on a viewer side with respect to the optical filter.

13. A manufacturing method for an optical filter, comprising a step of irradiating the light absorption filter according to claim 1 with an ultraviolet ray to carry out mask exposure.

14. A light absorption filter comprising:
a dye having a main absorption wavelength band in a wavelength range of 400 to 700 nm; and
a resin composed of a polymer including a partial structure that generates a radical upon ultraviolet irradiation.

15. The light absorption filter according to claim 14,
wherein the dye includes an electron-donating quencher-embedded coloring agent.

16. The light absorption filter according to claim 15,
wherein the electron-donating quencher-embedded coloring agent includes a squaraine-based coloring agent represented by General Formula (1A), 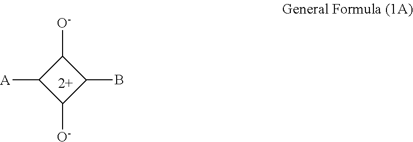
in the formula, A and B each independently represent an aryl group which may have a substituent, a heterocyclic group which may have a substituent, or —CH=G,
where G represents a heterocyclic group which may have a substituent, provided that at least one of A or B includes an electron-donating quencher moiety.

17. The light absorption filter according to claim 14,
wherein the polymer is a (meth)acrylic polymer having an aromatic ring in a side chain.

18. The light absorption filter according to claim 14,
wherein the compound that generates a radical upon ultraviolet irradiation is a compound that generates a radical upon intramolecular cleavage, and
the partial structure that generates a radical upon ultraviolet irradiation is a partial structure that generates a radical upon intramolecular cleavage.

19. The light absorption filter according to claim 14,
wherein the compound that generates a radical upon ultraviolet irradiation is a compound that generates a radical by extracting a hydrogen atom from a compound that is present in a vicinity of the compound, and
the partial structure that generates a radical upon ultraviolet irradiation is a partial structure that generates a radical by extracting a hydrogen atom from a compound that is present in a vicinity of the partial structure.

20. The light absorption filter according to claim 19,
wherein the compound that generates a radical by extracting a hydrogen atom from a compound that is present in a vicinity of the compound is a benzophenone compound substituted with an alkoxy group, and
the partial structure that generates a radical by extracting a hydrogen atom from a compound that is present in a vicinity of the partial structure is a partial structure derived from a benzophenone compound substituted with an alkoxy group.

21. The light absorption filter according to claim 14,
wherein in the light absorption filter, the dye is chemically changed to be decolorized upon irradiation with light.

22. An optical filter that is obtained by subjecting the light absorption filter according to claim 14 to mask exposure by ultraviolet irradiation.

23. A self-luminous display device, an organic electroluminescent display device, or a liquid crystal display device, comprising the optical filter according to claim 22.

24. The self-luminous display device, the organic electroluminescent display device, or the liquid crystal display device, according to claim 23,
wherein a layer, which inhibits light absorption of the compound that generates a radical upon ultraviolet irradiation or light absorption of the partial structure that generates a radical upon ultraviolet irradiation, is provided on a viewer side with respect to the optical filter.

25. A manufacturing method for an optical filter, comprising a step of irradiating the light absorption filter according to claim 14 with an ultraviolet ray to carry out mask exposure.
